SEQUENCE LISTING (100 sequences)

SEQ ID NO: 1 - DNA, 1605 bp, Bacillus licheniformis DSM 13
  gene (1)..(1605): yqjN - unknown function - similar to amino acid degradation - BLi02550
gtgaagtggcagacagtggaagaactgaaacggctgctcatttctcttgttcaatacgaaagcatctcaggcacggccggagaagtggcgcttgcgaaatatatgcatgatgtcttaaaggaccgttcgtactttcaaaagaatcctgaatacttaaagcttcacccgatggaggatgggcgctattttttaaccgccctcgtcaaaaaggaaaagaaatcaaatacggtgctcctcttgagccattttgacgtggtcgatacggcagactacggagaattcaaacatatggcatgcaaagtcccggaactcatggacctattaaatgataaaaaagaacttttgcctgaacgggtcaaaaaagacctggaaagcggcgaatggctgttcggtaggggaagcatggatatgaaagcgggtcttgctgttcaattttctatgctggaacgggcgatgaacggagaatttgacggaaaccttctattaattacggttccggacgaagaagtcaactcgcaggggatgcttgaagcagtacctgtactgaaagctttagaaaaagaatttggccttcattatacagcgtgcttaaactgtgagccgatgtttgaaaaacatccgggtgatccgaagcagtacgtctattcaggcagcatcggcaaagcgcttgccggatttttctgcaaaggggttgagacgcatgtcggcgagccgtttgcaggggtgaatgcgaatctaatggtgtctgagatcaacaggctgctcgaattaaacgtcgactattgcgaagaagcggacggagaggtgacgcctcccccggccaatctgatgcaaaaagatctgaaggaggcctactcggttcagactcctcacacagcggtgtcgctctttaatgtgctgatgatgcagcgttccgccgccgagctccatcgcatgctgctcgacacagccagggaagcggccgccaatattcaaacaaatatcgaacgtaaaacggccgaatatcagcggtttgaagccttcacgccaattaacccggccgtttccgtgatgacgtttgaggagctgtggaacaaagcacaggaaaaagccgggaagcccgaggcggaacgcatcctcaactttgcatttgcgaacaggggggagcttggagaccgcgacttttcaacgaaaatcgtctctgaattggcttcactctgcaaagaagatgctccgatgatcgttctgttttacagtccgccgttttatccggctgtttcttcaaaggacgatcctctgatccgaaaggtgatgagagaagtgaagcagtatgcggccgacaggcacggaattgaatttacagaagtccaatatttccccggcctgtccgatttaagctacctgcagctgcaaaaacagtcagtcgatgcgttcacatccaatatgccgctttttcatcacggctattcacttccaaaaggacgcgaaaacgcactgtttgtgccggtcatcaatatcgggccggccggcaaagatcctcacaaatggacggaacgtctccatgtgccgttttcatttggaaagctgccagatatcctccggctcgcgatacacaagcttctctaa

SEQ ID NO: 2 - protein, 534 aa, Bacillus licheniformis DSM 13
  misc_feature (1)..(1): Xaa can be Val or Met.
XKWQTVEELKRLLISLVQYESISGTAGEVALAKYMHDVLKDRSYFQKNPEYLKLHPMEDGRYFLTALVKKEKKSNTVLLLSHFDVVDTADYGEFKHMACKVPELMDLLNDKKELLPERVKKDLESGEWLFGRGSMDMKAGLAVQFSMLERAMNGEFDGNLLLITVPDEEVNSQGMLEAVPVLKALEKEFGLHYTACLNCEPMFEKHPGDPKQYVYSGSIGKALAGFFCKGVETHVGEPFAGVNANLMVSEINRLLELNVDYCEEADGEVTPPPANLMQKDLKEAYSVQTPHTAVSLFNVLMMQRSAAELHRMLLDTAREAAANIQTNIERKTAEYQRFEAFTPINPAVSVMTFEELWNKAQEKAGKPEAERILNFAFANRGELGDRDFSTKIVSELASLCKEDAPMIVLFYSPPFYPAVSSKDDPLIRKVMREVKQYAADRHGIEFTEVQYFPGLSDLSYLQLQKQSVDAFTSNMPLFHHGYSLPKGRENALFVPVINIGPAGKDPHKWTERLHVPFSFGKLPDILRLAIHKLL

SEQ ID NO: 3 - DNA, 351 bp, Bacillus licheniformis DSM 13
  gene (1)..(351): nrgB - nitrogen-regulated PII-like protein - BLi03889
atgagcggtcaaatgtataaagtggaaattgtaacccgtcctacaaactttgaaaagctgaaaacggagctggccaaaatcggggtaacgtccattacgttttacaatgtacacggatgcggcctgcaaaaagggcatactgagctttaccgcggggtcaaacgcgaaagcaatgtgtatgaaaggttaaaagtcgaaatcgtcgtcagcaaagtgcctgtcgaaaaggtggccgagacggcgcaaaacgtcctgaaaaccggggaacccggtgacggcaagatcttcatatacgaaattaaaaacaccatcaacatccgcacaggcgaggaaggtcctgacgctttataa

SEQ ID NO: 4 - protein, 116 aa, Bacillus licheniformis DSM 13
MSGQMYKVEIVTRPTNFEKLKTELAKIGVTSITFYNVHGCGLQKGHTELYRGVKRESNVYERLKVEIVVSKVPVEKVAETAQNVLKTGEPGDGKIFIYEIKNTINIRTGEEGPDAL

SEQ ID NO: 5 - DNA, 1212 bp, Bacillus licheniformis DSM 13
  gene (1)..(1212): nrgA - ammonium transporter - BLi03888
atgcagatgggggatacagtatttatgttcttttgcgctttgctcgtgtggctgatgacgccgggcatcgctttattttacggaggccttgtaaggagcaaaaatgtgctcagcaccgttatgcacagtttatcttcaattgccatcgtttctatcatatggattttgttcggctattcactcgctttcgctcccggaaacccattgatcgggggtttggattggatcggactgcacggtgtcggttttgaacccaatgccgattattcggagacgatcccgcatacgctttatatgatgttccaattgacatttgccgtcctgacgactgcgattatttccggaagctttgcggagcgcatgcgctttccggcctttattcttttctcggtgctctgggcggttttcgtttattcgcctgtcgcccactgggtatggggcggaggctggcttgacgagctgggagctattgattttgccggcggaaacgtcgttcacatttcttccggggtagccggtctggtcgttgcgattgttctcggcaagcggaaaaatatgagcgacaccgcccctcacaacctccttctgaccctgctcggggcgactttgatctggttcggatggttcggtttcaatgtcggaagcgcgctgacgatcgatgaagtcgccatgaccgcttttatcaacacgaatacggctgcggccgcggggctgatcggctggattttggcagaatggctgattaataagaagccgacgatgctcggcgccgtctcgggagcgattgcgggactcgtcgcgattacgcctggcgcaggttttgtcacgccattctcatctgtatggatcgggcttatcggaggaatcgtctgtttctggggggtgttttcgctcaagaaaaaattcgggtacgatgatgcgctcgatgccttcggacttcacgggctcggcgggacatggggcggaatcgcgacaggtttgttcgcgacgacatctgtcaacgcagacggtgccaacgggcttttttacggagacatcagtctcctttggaaacagctgattgcgattgctgcaacatatttgtttgtcgcaatcgtcacatacgctattattaagatagtcagtatcttttttaaactccgtgcctcagaggatgaagaatcattgggcctcgatcttacgctgcacggcgaaaaagcgtatcaagattaa

SEQ ID NO: 6 - protein, 403 aa, Bacillus licheniformis DSM 13
MQMGDTVFMFFCALLVWLMTPGIALFYGGLVRSKNVLSTVMHSLSSIAIVSIIWILFGYSLAFAPGNPLIGGLDWIGLHGVGFEPNADYSETIPHTLYMMFQLTFAVLTTAIISGSFAERMRFPAFILFSVLWAVFVYSPVAHWVWGGGWLDELGAIDFAGGNVVHISSGVAGLVVAIVLGKRKNMSDTAPHNLLLTLLGATLIWFGWFGFNVGSALTIDEVAMTAFINTNTAAAAGLIGWILAEWLINKKPTMLGAVSGAIAGLVAITPGAGFVTPFSSVWIGLIGGIVCFWGVFSLKKKFGYDDALDAFGLHGLGGTWGGIATGLFATTSVNADGANGLFYGDISLLWKQLIAIAATYLFVAIVTYAIIKIVSIFFKLRASEDEESLGLDLTLHGEKAYQD

SEQ ID NO: 7 - DNA, 1302 bp, Bacillus licheniformis DSM 13
  gene (1)..(1302): htrA - serine protease Do (heat-shock protein) - BLi01390
atgatggaatataatagagaagatcagcttgaaacaaaaaacgaactcatcgtctccgatcaaaagcccgaccaggcgataaaaaaagcagaacagcaaaagcggagactctcgtggatgacgccgattctcagcggcatcatcggaggaagcctcgttctcggcgtcacgacataccagcatagcaaagacgaaacggccaatacatccgtccagacgcaaacaaacgagacgtcgaactcatcggcttcttccactaaagcgaataccgaaaaattgtccagcaccaattcgtccgacatttctacaatggttgaaaatgtatctcccgccatcgtcggtatttcaaactatcaaaaacaaacagagagcggtatgttcggttttgaaagcaacagcagcagctcaagcgaacaagaaacaggcacaggctcaggtgtcatctataaaaaagcaaacggcaaagcatacattattacaaataaccacgttgtcgaaggcgcgtctaaactgacggtttccctgtccaacggcaaagaagtggaaggtaaattgctgggcagcgattcgttgacagaccttgccgttgttgaaatttccgctgatcacgtggaaaaagtcgcctctttcggcgattcgtcaagcttgaaagcaggcgaatcggtgatcgctattggaaacccgctcggcaaagatctgtcccgcacggtgacacaaggcatcatcagcggtgttgatcggaccgtttccgtagatacatccgcaggccaaaccgagatgaacgtcattcaaaccgacgcggccatcaaccccggcaacagcggaggcgcccttttgaatacaaagggtgaagtgatcggcatcaacagcatgaaaatcagtgaaagcggagtagaaggcatcggatttgcgattccaagcaatgatgtcaaaccgattgcagaagagctgatgtctaaaggaaaaattgagcgcccgttcatcggaatcggcatgatggatcttgagcaggtgccggaaaactatcaaacaggcaccctcggattaagcggcagccagttaaacaaaggcgtctacatccgccaggtcgcttcaggctccccagctgaaaaagcgggccttaaagagaacgatgtcatcgtcagctttaacggaaaagaaaccgatacaggaagcgcgctgcgcaaccttctttacaacgatgcaaaaatcggggatacggtaaaggttaccctcattcgaaacggcaaaaccatgacaaaacaaatcacccttgaccagaaagaatccgcatcatcttaa

SEQ ID NO: 8 - protein, 433 aa, Bacillus licheniformis DSM 13
MMEYNREDQLETKNELIVSDQKPDQAIKKAEQQKRRLSWMTPILSGIIGGSLVLGVTTYQHSKDETANTSVQTQTNETSNSSASSTKANTEKLSSTNSSDISTMVENVSPAIVGISNYQKQTESGMFGFESNSSSSSEQETGTGSGVIYKKANGKAYIITNNHVVEGASKLTVSLSNGKEVEGKLLGSDSLTDLAVVEISADHVEKVASFGDSSSLKAGESVIAIGNPLGKDLSRTVTQGIISGVDRTVSVDTSAGQTEMNVIQTDAAINPGNSGGALLNTKGEVIGINSMKISESGVEGIGFAIPSNDVKPIAEELMSKGKIERPFIGIGMMDLEQVPENYQTGTLGLSGSQLNKGVYIRQVASGSPAEKAGLKENDVIVSFNGKETDTGSALRNLLYNDAKIGDTVKVTLIRNGKTMTKQITLDQKESASS

SEQ ID NO: 9 - DNA, 1215 bp, Bacillus licheniformis DSM 13
  gene (1)..(1215): putative protein - putative serine protease- BLi00301
ttgaaatcgaaatggagtgcaatggtggttattgccggtcttttattgctggccggatgcggtgcactgaaggaggctgatcccccgcgcggcagatcagcgcaaaaaacggaagcctcgttttctgaagctgaacagcgatttgcgctcgccttgtttcaagacatgataaaagaagaagggagccggaaaaacatcttcctctcgccttacagtattcagcaggcacttttgatgacggcaaacggtgccgcgggagacagcagaaaggaactgctcagcactttacatctcagccaggcggatatggcatcgatcaacgggatatcaaaatctgttaatcgttctcttgaaacgctgcctcacggtgaattttcatcagctaattcgatatggaccaaaagggagctgaaagaaagctttcaagacgcgatcaaaggccttggcgacgcctatcagcttcaaattgatcctaatcgggcaacagaggaaatcaacagctgggtcaaaaagaaaacgaaagaccgcatcgatcagatcgttgataaagtctcgtcgaatacgattgcctacatcatcaatgccgtctattttaaagaaagctggaagcacccatttgatcctcacatgacagcggaacagccattttattcagcggacggctccgcaaaaaaacaccccatgatgacgcagacaaaccgctttccttatctggaaaacgaacattttcaagccgtcaagctgccatttcaagacgaaggcctgtcactagccgtttttctgccgaaaaaagcggcaaatctagaacagcttctgactggcatgacgcatgatcaatggcgcgccttgcaaaagggctggacgatgaaacaggtggagctcaagctgccgcgcttctcatttcaaacagactacatgttaaatgaaccgcttaaacgccttggaatgaaaaacgtgtttagcagcgcgaatttttcaaatatgtttaccgggcatggcgccgctcaaattaataaagtcagacataaaacatttatcaaagtggatgaagcgggcactgaagcatccgcggcaacggcggttgaaatcatcgaatcagcccccgtccctgaagtgaccatgactgcagaccacccgttttattttgcgatcgttgatgaagcatccggaatgattctctttttgggcagtgtggcagagccgaaagatgattaa

SEQ ID NO: 10 - protein, 404 aa, Bacillus licheniformis DSM 13
  misc_feature (1)..(1): Xaa can be Leu or Met.
XKSKWSAMVVIAGLLLLAGCGALKEADPPRGRSAQKTEASFSEAEQRFALALFQDMIKEEGSRKNIFLSPYSIQQALLMTANGAAGDSRKELLSTLHLSQADMASINGISKSVNRSLETLPHGEFSSANSIWTKRELKESFQDAIKGLGDAYQLQIDPNRATEEINSWVKKKTKDRIDQIVDKVSSNTIAYIINAVYFKESWKHPFDPHMTAEQPFYSADGSAKKHPMMTQTNRFPYLENEHFQAVKLPFQDEGLSLAVFLPKKAANLEQLLTGMTHDQWRALQKGWTMKQVELKLPRFSFQTDYMLNEPLKRLGMKNVFSSANFSNMFTGHGAAQINKVRHKTFIKVDEAGTEASAATAVEIIESAPVPEVTMTADHPFYFAIVDEASGMILFLGSVAEPKDD

SEQ ID NO: 11 - DNA, 408 bp, Bacillus licheniformis DSM 13
  gene (1)..(408): glnR - transcriptional repressor of the glutamine synthetase gene- BLi01992
atgagtgataaaattcgccgctcaatgcccttatttccgataggtatcgtcatgcagctgactgaactgtccgcaaggcaaattcggtattatgaggaaaatggactggtatttcctgcaagaagcgaagggaacagacgtttattttcattccatgatgtagataagctgctcgaaattaaagatttgatcgaacaaggtgtaaacatggcggggatcaaacaaattttcgccaaaacagaaaacgaacaaaaagaagacaaacaagacaagccgaaaaaagcagagaagcacaacttgtcggatgaagaactgagaaaactgctgaaaaacgagctcatccaagcaggccgtttccaaagagggacgacattccgccagggagacatgtccaggttttttcattaa

SEQ ID NO: 12 - protein, 135 aa, Bacillus licheniformis DSM 13
MSDKIRRSMPLFPIGIVMQLTELSARQIRYYEENGLVFPARSEGNRRLFSFHDVDKLLEIKDLIEQGVNMAGIKQIFAKTENEQKEDKQDKPKKAEKHNLSDEELRKLLKNELIQAGRFQRGTTFRQGDMSRFFH

SEQ ID NO: 13 - DNA, 1335 bp, Bacillus licheniformis DSM 13
  gene (1)..(1335): glnA - glutamine synthetase - BLi01993
atggcaaagtatacaagagacgacattgttaaactagtaaatgaggagaatgtaaaatatattcgccttcagtttacagacattcttggaacgattaaaaacgttgaaattcctgtgagccagctggaaaaagcacttgataataaagtcatgttcgacggctcttcaatcgagggattcgtccgcattgaagaatccgatatgtacttataccctgacttagatacatttgtgattttcccatggaccgccgaaaaaggtaaagtagcccgcttcatctgcgatatttataatccggacggcacgccgtttgaaggtgatccgcgcaacaacttaaaacgcattttgaaagaaatggaagacatgggcttctcagatttcaatcttggaccagagccggaattcttcctgttcaagctagacgaaaaaggcgagcctacgcttgaactgaacgacaaaggcggatatttcgaccttgcgccgactgatcttggcgaaaactgccgccgcgacatcgtacttgagctcgaagaaatgggctttgaaatcgaagcgtctcaccacgaagttgcacctggacagcacgaaatcgacttcaaatacgccggagccatccgcgcttgcgatgacatccaaacgttcaagctcgttgttaaaacaatcgcgcgcaaacacggcctgcacgcaacattcatgccaaaaccgttgttcggcgtaaacggatcaggcatgcactgcaatctatcactcttccgaaacggcgccaacgccttctttgacaaagacgccgatcttcagctgagtgaaacggcaaagcaattcatcgccggcatcgtcaagcacgcaacaagctttacagctgtgacaaacccgactgtcaactcttacaagcgccttgttccaggctatgaagcaccttgctatgtggcatggagcgcgcaaaacagaagcccgttaatccgcatcccggcttcccgcggcatcagcacgcgcgtagaagtcagaagcgtagacccttctgcgaacccgtaccttgccttgagcgtacttcttgcagcaggacttgacggcatcaaaaacaagctggaagcaccggctccgatcgaccgcaacatctatgtcatgacaaaagaagagcgcctcgaaaacggaatcgtcgaccttcctgcgacactcgcagacgctctcgaagaattcaaatcaaacgaagtcatggtcaaagccctcggcgaccatctattcgaacacttcgtcgaagcaaaagaaatcgaatgggatatgttccgcacacaagtccatccttgggagcgcgagcagtatatgtctcagtattaa

SEQ ID NO: 14 - protein, 444 aa, Bacillus licheniformis DSM 13
MAKYTRDDIVKLVNEENVKYIRLQFTDILGTIKNVEIPVSQLEKALDNKVMFDGSSIEGFVRIEESDMYLYPDLDTFVIFPWTAEKGKVARFICDIYNPDGTPFEGDPRNNLKRILKEMEDMGFSDFNLGPEPEFFLFKLDEKGEPTLELNDKGGYFDLAPTDLGENCRRDIVLELEEMGFEIEASHHEVAPGQHEIDFKYAGAIRACDDIQTFKLVVKTIARKHGLHATFMPKPLFGVNGSGMHCNLSLFRNGANAFFDKDADLQLSETAKQFIAGIVKHATSFTAVTNPTVNSYKRLVPGYEAPCYVAWSAQNRSPLIRIPASRGISTRVEVRSVDPSANPYLALSVLLAAGLDGIKNKLEAPAPIDRNIYVMTKEERLENGIVDLPATLADALEEFKSNEVMVKALGDHLFEHFVEAKEIEWDMFRTQVHPWEREQYMSQY

SEQ ID NO: 15 - DNA, 1476 bp, Bacillus licheniformis DSM 13
  gene (1)..(1476): hypothetical protein - close homolog to DhaS aldehyde dehydrogenase - BLi03994
atgaatgttcaaactccatcggaaaaactcgtaaaaccttttgactgtcagcactacataaacggcaggtactttccttcagagaaaggcagcacgtttgacaacatcaatccggctacacaggaaattatcggaagcgttgcggaaggaggcaaaaaagaggtcgatcttgcagtcgcggcagccaggcgtgcattaaacggacgctggaaaaacatgaccgctgacgaaaggattcggatcatcagaaaggtcggcgacatcatacttgagcgtaaagacgaactggcgaggcttgagacattggatacgggaaaaccgctttcgctctcaagcactttggatattccacgtgccgctttcaactttcatttctttgcggattttatgagaggagccggaacggaagcttatcaaactgacgatcttgcaatcaattatgcgattcgccgtccggtcggcgtcgtcgggctgatcaatccgtggaacttgccgcttcttttgctgacgtggaaattggcgccttgcctggcggcgggaaatacagccgttatcaaaccggctgaactgacgccgatgacagccacccttctcggggaaatctgccatgatgcagggatgcctgacggagtcgttaatatcgtccacggatttggaccagattccgccggtgctgcgctttctgagcatcccgatgtcgatgcgatatctttcaccggagaaacgacaaccggcaaagtcattatggaagctgcttcgaaaacattaaagaaactttcctttgaattgggagggaagaacccgaacattatttttgccgatgccgacatggatgaagccgtcagcacctcattgaaatcgagtttcgtcaaccagggggaagtatgtatgagcggctcccgcatctatgtcgagagggaagcgtatgacgaatttctgaacaagttcgttgagaaaacgaaaaagctcaaagtcggcgatccttttgatccggacacgaatatcggcgccttgatttcttctgaacacacagagcgtgtgatgaactacatcgaattggccaaacgggaaggcggcaatattttaaccggaggaaaacgccctgaaggacaggaatccggctgctttcttgaaccgacgattatcaccggactttcacgcaattccaggctcatcaaagaagaaatcttcggtccggtcgtaacggtgattccgttcgacgatgaggaggaagtgattgcacaggctaacgacacacattacgggttaagcgcgaccttatggacgaacgacctccgccgggcgcaccgcgtagccgggcaaatcgaatcgggcatggtgtgggtcaacagctggtttctccgcgatcttcggacgccgtttggcggcatgaaacagagcggactcggacgagaaggaggcattcacagcctcgaattcttcagcgaattaaccaatatttgcatcaagctgtaa

SEQ ID NO: 16 - protein, 491 aa, Bacillus licheniformis DSM 13
MNVQTPSEKLVKPFDCQHYINGRYFPSEKGSTFDNINPATQEIIGSVAEGGKKEVDLAVAAARRALNGRWKNMTADERIRIIRKVGDIILERKDELARLETLDTGKPLSLSSTLDIPRAAFNFHFFADFMRGAGTEAYQTDDLAINYAIRRPVGVVGLINPWNLPLLLLTWKLAPCLAAGNTAVIKPAELTPMTATLLGEICHDAGMPDGVVNIVHGFGPDSAGAALSEHPDVDAISFTGETTTGKVIMEAASKTLKKLSFELGGKNPNIIFADADMDEAVSTSLKSSFVNQGEVCMSGSRIYVEREAYDEFLNKFVEKTKKLKVGDPFDPDTNIGALISSEHTERVMNYIELAKREGGNILTGGKRPEGQESGCFLEPTIITGLSRNSRLIKEEIFGPVVTVIPFDDEEEVIAQANDTHYGLSATLWTNDLRRAHRVAGQIESGMVWVNSWFLRDLRTPFGGMKQSGLGREGGIHSLEFFSELTNICIKL

SEQ ID NO: 17 - DNA, 780 bp, Bacillus licheniformis DSM 13
  gene (1)..(780): putative protein - putative transcriptional regulator - BLi03995
atgactgttgagacgaaggaccaccagctgctcggttcagtaaaaaatgcgcttcggctgcttcaatcgtttaccttagacacaccggaaaagaaactaaccgaactcgcctcgtcgcttggcctcggaaaaagcacggtctcgcgccttttgtctacattggaaagcgaagggtttgtcatgaaggacccggaatccagaagatacaagctcggcttatcggtcctgcagttaaacaccattgtcaactccactcttgaaatcaaccgcgaatccctgccggtcctcaaacggctgaccgatgagaccggcgaaacgtcgcatatcgcgatgcgccgcggtctcagcgtcgtttatctcaatcggacggaatgcccccgcccggtcgaacttctctcatatatcgggcggcaaaacccgatgcactgcacaagctcaggtaaggtgcttctcgcctttgatgaagagggcatattggacgagtatgtgaaggaaggcttgaaaagctgtacgaaagaaaccattacagacggggacaaattgagggatgtgctgaaaaccgttaaagaaaacggatttgacgtcagttgcggggaattcatagatggtgtcacatcaatctcagctcccgtcagaaaccacgcaggtcgagtgttgtacgccgtcagtgtcgtaggccctgcaaaacgcatcaaggcccggcaggcgaaaatcatttaccaagtgaaacaagcggcccgggatatttccgaaaggatcgggtactggaaacgaatataa

SEQ ID NO: 18 - protein, 259 aa, Bacillus licheniformis DSM 13
MTVETKDHQLLGSVKNALRLLQSFTLDTPEKKLTELASSLGLGKSTVSRLLSTLESEGFVMKDPESRRYKLGLSVLQLNTIVNSTLEINRESLPVLKRLTDETGETSHIAMRRGLSVVYLNRTECPRPVELLSYIGRQNPMHCTSSGKVLLAFDEEGILDEYVKEGLKSCTKETITDGDKLRDVLKTVKENGFDVSCGEFIDGVTSISAPVRNHAGRVLYAVSVVGPAKRIKARQAKIIYQVKQAARDISERIGYWKRI

SEQ ID NO: 19 - DNA, 951 bp, Bacillus licheniformis DSM 13
  gene (1)..(951): conserved hypothetical protein - unknown function - BLi03996
ttgtatgatcttcacacgcactttataccgcgcgatgtgctgctttggctggaagacaatgaaaagagagtccacgctgtccgcgaaaaaaaggctggatgccaagcggagtttttgacggtcaatcacaaatggggctttgaattaaaagagcttttctaccaggaggctttatatttggaagcccagcaggaggccggcgtcacacactcattggtatcgccggttccccagcttttcctttacgatctcgccgaagaaatcacggacgaattatcttttgtatacaacaaagcgcttgccgaatgggttaaaaaatacgacggccgcctctcgggactcggcacagtcccgctgaacagcccttcaaaggcgagcgagcgcctgaatgaagcgatgaacaacgggcttaaaggagcgattatcggacccggcgcttcgcaactacttctctcggacgaagcatttacaccgttttgggaaacggctgatagaagaaaagcgattatctttatacatccgctcctttctgaagatccgcggctccgccgcaggatgctgccaaacttgatcggcgttccgtgggaaacgacgatttgtgccgcagaccttttgttaagcgggtttacagaccggtttcgcaatgtgaaaattcttttggcccacggaggcggctttttgccttatcagatcggaagactgaacaaagggtatgacaaatgggccggcgtttcttcatcgctttcggctccgccgcgcgaatatttgcggaagttttggtttgatggtgtgctatgggagcaagaaagcgcggcatatttgaagaagcttgtcggagaagaccgccttgttccgggttccgattttcctttcgacctttgtcagtggccgcctctcgatcccggccggaaaggagcggagtctctgctgaatgtagcgattgaagcataa

SEQ ID NO: 20 - protein, 316 aa, Bacillus licheniformis DSM 13
  misc_feature (1)..(1): Xaa can be Leu or Met.
XYDLHTHFIPRDVLLWLEDNEKRVHAVREKKAGCQAEFLTVNHKWGFELKELFYQEALYLEAQQEAGVTHSLVSPVPQLFLYDLAEEITDELSFVYNKALAEWVKKYDGRLSGLGTVPLNSPSKASERLNEAMNNGLKGAIIGPGASQLLLSDEAFTPFWETADRRKAIIFIHPLLSEDPRLRRRMLPNLIGVPWETTICAADLLLSGFTDRFRNVKILLAHGGGFLPYQIGRLNKGYDKWAGVSSSLSAPPREYLRKFWFDGVLWEQESAAYLKKLVGEDRLVPGSDFPFDLCQWPPLDPGRKGAESLLNVAIEA

SEQ ID NO: 21 - DNA, 921 bp, Bacillus licheniformis DSM 13
  gene (1)..(921): putative protein - putative phage capsid protein - BLi01470
atgagttttgatccgaataacgtattgatgcaagatgcagtcaaaggaaaagtaccatctgatcaaggaacattagtattaaaagactttatgacacaatcagcagtcacaaaactagcaaaatatgaagaaatggataaaaccgagaagacattcacttatcttgcgtctggccccggggcttactgggttggcgaaggtgagagaatcaaaacttctaaggctacatggttagaagcgaaaatgacctctaaaaaactcggggtcattgtcccagtcacaaaagagtttttgaattactccgtgaaagacttctttactcaaatgcggccagcaatcgcggaagcattcgctattaaatttgaccaagcagcactttttggcattgattcaccattcggaaaaggcacttctgtattcgaaagagcagaagcagcaggaaacactgttgttttgaactctctaggcaacctttacgatgagttaaacgcagtcatggcactcactgaggacaatgacaaagacgtgaacggctttactactacacgtcgtttcaaacaaaaactacgcggcactaaggacggcaacgggctgcctatttttaatgatgcaagaggcggcgcgacatcagaggcattaggtttgccgattggctatgtcgattctaagtcttgggattatacaaaagcacatctattggctgccgattgggattttacccgttacggaattcctcaaggtatggaataccatatttcgcaagatgcaactttaacaacggttgtcgatgaaaataacaatcctatcaacttgtttgagcgtgatatgtttgctctccgagtgactcaacaagtcggatttatgacactttctgacgaagcattcgcagctcttactccggaagcagaagcgggggcgtaa

SEQ ID NO: 22 - protein, 306 aa, Bacillus licheniformis DSM 13
MSFDPNNVLMQDAVKGKVPSDQGTLVLKDFMTQSAVTKLAKYEEMDKTEKTFTYLASGPGAYWVGEGERIKTSKATWLEAKMTSKKLGVIVPVTKEFLNYSVKDFFTQMRPAIAEAFAIKFDQAALFGIDSPFGKGTSVFERAEAAGNTVVLNSLGNLYDELNAVMALTEDNDKDVNGFTTTRRFKQKLRGTKDGNGLPIFNDARGGATSEALGLPIGYVDSKSWDYTKAHLLAADWDFTRYGIPQGMEYHISQDATLTTVVDENNNPINLFERDMFALRVTQQVGFMTLSDEAFAALTPEAEAGA

SEQ ID NO: 23 - DNA, 1371 bp, Bacillus licheniformis DSM 13
  gene (1)..(1371): yvtA - unknown function - similar to HtrA-like serine protease - BLi03481
atggattttagacgcgatgacgaacaaaaacatgcaaatgaagagcagtttcacgaagaagcgcagacgccgcaaacggcaggttctgaggagcagcagtcagaacagccggcaaaggagcccgagctgattttaaagaaggatacagacgatcagtcagatcttacgaataaagaaaccgctgcagccctggaagatgccagggaaacgagagcggcaaaagaaaagcggcgcaaggcttcgtggctgagcccgattttgggcgggatcatcggaggcggcttggtgcttggaatctccccgtatctgccgggcgatcatccgagcgaaactgccggaaccggacagacagagccaaagcagtcgcagaatttcaccacaaaacctgtcacgaacgcagataacgtacctgatatggtggaagatttggagcctgcgatcgtcggagtttcaaatattcaaacgagcttcggcttctctgaagacgacgtggaggagagcggcacgggatcaggcgtcatctttaagaaagacggcggcaaggcctacattatcacaaacaaccatgtcgttgaaggcgcctcgaaggtgaccatctccttatacaacggaaagaccgcggatgccaagattatcggcagcgacgctttgactgatctggctgttttagagatcagcagcaaaggcgttgataaggttgcaagtttcggagactccgcaaaactgcgcgccggcgaaaaagtaattgcgatcgggaaccctctcggccttcaattttcaagaacggttacagaaggcatcatcagcggtgtcaaccggacgattgaagtctcaacatctgaagggaattgggatatgaacgtccttcagacagatgcggcgatcaaccctggaaacagcggcgggcctctgattaacagcagcgggcaggtcatcggcatcaacagcctgaagatcagccaaagcggtgttgaatcgcttggcttcgcgattccgagcaacgatgttcagccgatcgttgacgagcttttggaaaaagggaaagtggaacggcctttcctcggcgtgcaaatgatcgatatgcagcaggtgcctgaacagtaccagcaaaatacgctcggcctattcggagaccagcttaataaaggcgtgtacatcgataaagtatcgccgaaatcgcctgcggcagacgcgggtatgaaagccggcgacgtcatcaccaaaatgaacggcaaaaacgtcgaaaccacctcagacctcagaaaaattctttatacagaagcaaaagcaggagatactgtcacatttgaggtgctgagaaacggaaagcaaacaacaatgaaagcaaaacttgcgaaaagcaaatcataa

SEQ ID NO: 24 - protein, 456 aa, Bacillus licheniformis DSM 13
MDFRRDDEQKHANEEQFHEEAQTPQTAGSEEQQSEQPAKEPELILKKDTDDQSDLTNKETAAALEDARETRAAKEKRRKASWLSPILGGIIGGGLVLGISPYLPGDHPSETAGTGQTEPKQSQNFTTKPVTNADNVPDMVEDLEPAIVGVSNIQTSFGFSEDDVEESGTGSGVIFKKDGGKAYIITNNHVVEGASKVTISLYNGKTADAKIIGSDALTDLAVLEISSKGVDKVASFGDSAKLRAGEKVIAIGNPLGLQFSRTVTEGIISGVNRTIEVSTSEGNWDMNVLQTDAAINPGNSGGPLINSSGQVIGINSLKISQSGVESLGFAIPSNDVQPIVDELLEKGKVERPFLGVQMIDMQQVPEQYQQNTLGLFGDQLNKGVYIDKVSPKSPAADAGMKAGDVITKMNGKNVETTSDLRKILYTEAKAGDTVTFEVLRNGKQTTMKAKLAKSKS

SEQ ID NO: 25 - DNA, 1017 bp, Bacillus licheniformis DSM 13
  gene (1)..(1017): kdgR - transcriptional repressor of the pectin utilization operon - BLi03828
atgaaggataagcaaacagcgaaagtaaccatcaacgaagtcgccgcacatgcaggcgtatcaaaatcaacggtttcccgttatatcaacgggaagattgatgccatttcaccgaaaaaggtgaaaagcatcaaaaaagcgatcgaggagctcaattaccgcccaagccagatggcacaggggctgaaggtgaaaaaaagcaaagttatcggatttttggtcgccgatattacaaacccgttttcagtagccaccttgagaggggtggaagaggtttgcgatcagtacggatacagcatcatggtctgcaacacggacaacagtcctgagaaagaaagggagatgctgcacaagctgaacgcgcattacattgaagggctgattattaatacgacgggaaggaacaacgatgtgctcttggatttaatcagccaggatgtatcgattgtgctcgttgaccggaaagtgcccgggctcaagatcgatgcagtcacaacgaacaaccgcgaagtgacaagtcaaatcgtcaatctcatgtatgacaaaggctatgcccaggtcggtctgtttaccgaaccgattaagggaatcagtccaagggaggaaagggcctccgcctatacagaaatcgcgctggaacgaaacgccggcggtacccctctcgtatatgaggctgacgtcaaagacaaagaatctctattacagtccgtcaaaagctttttggaaatgaaagaagggaaaaaagccattcttggaaacaacgggcttctgatgctgaaaatcatcagctgcctttatgagcttggcatttcaattccggaagatgtcggaattgccggatttgatgatacagaatggtataaattgatcggtccgggcattacgaccatcgcccagccgtcgcatgaaatgggcagggtcgccatggagcggatcatcaaacggattgaaggagacgaaagcgccccgcagacgattcagcttgaggcggagcttgttctcagacagtcgctgtag

SEQ ID NO: 26 - protein, 338 aa, Bacillus licheniformis DSM 13
MKDKQTAKVTINEVAAHAGVSKSTVSRYINGKIDAISPKKVKSIKKAIEELNYRPSQMAQGLKVKKSKVIGFLVADITNPFSVATLRGVEEVCDQYGYSIMVCNTDNSPEKEREMLHKLNAHYIEGLIINTTGRNNDVLLDLISQDVSIVLVDRKVPGLKIDAVTTNNREVTSQIVNLMYDKGYAQVGLFTEPIKGISPREERASAYTEIALERNAGGTPLVYEADVKDKESLLQSVKSFLEMKEGKKAILGNNGLLMLKIISCLYELGISIPEDVGIAGFDDTEWYKLIGPGITTIAQPSHEMGRVAMERIIKRIEGDESAPQTIQLEAELVLRQSL

SEQ ID NO: 27 - DNA, 645 bp, Bacillus licheniformis DSM 13
  gene (1)..(645): ycnI - unknown function - similar to unknown function proteins - BLi00479
atgttgaaaaagacattatggatgggtgcaatcctagcatcatttttgcttttctccgtgccggtcagcgcacatgtcacggtaaaaccgaccgaatcagcagcaggctcatgggaaacatacactgtcaaagtaccggttgaaaaagacaacgccacgatcaaggtcgttctcagcatgccggacggtgttgaatttcagcagtatgaaccggtgccggggtggaaaacaagcactgaaaccggcaaaaacgaaaaagtaacaagggtgacgtgggaagccgaaaacggcggcattcagcctggtgaattccagcagtttaccttcaccgccaaaaaccctgacaaagaacaaaaggccgcctggaacgcataccaatattataaggacggcacaattgtcgagtggaccggtgatgatgacgccgataccccgcatgcaatcacaagcattgtcgacgctgcaaaactcggacacgaaacggttgatgcacacggcgcttcaacgaaacaaaacgctgccgcagaaaatgaagacagtcaaggaggacaagggattcaaaccgcttccctgatcctgtcgatcctcgccgtgcttcttggaggtgctgcgctgctcttgacagtgcggaaaaagaaatcgtaa

SEQ ID NO: 28 - protein, 214 aa, Bacillus licheniformis DSM 13
MLKKTLWMGAILASFLLFSVPVSAHVTVKPTESAAGSWETYTVKVPVEKDNATIKVVLSMPDGVEFQQYEPVPGWKTSTETGKNEKVTRVTWEAENGGIQPGEFQQFTFTAKNPDKEQKAAWNAYQYYKDGTIVEWTGDDDADTPHAITSIVDAAKLGHETVDAHGASTKQNAAAENEDSQGGQGIQTASLILSILAVLLGGAALLLTVRKKKS

SEQ ID NO: 29 - DNA, 1647 bp, Bacillus licheniformis DSM 13
  gene (1)..(1647): ycnJ - unknown function - similar to copper export protein - BLi00480
atgaatgtgatgaaaagagcaacactctgggcagttctggctgttctttgtaccattctgctgccaaaagccgcttttgctcacgcttatatcgtacaatcaactcctgctgaaaatcaggagctgaaaaaagcgcctgaagaaattaagatcgaattcaatgaagcgattcagaacgggtttcattcgataacggtcagaaattcgtcgggtgaacgggtagattccggcaaaacggaaatcgacccccaaaacgcaaagatcatgaccgtcaagctgaaaccgaagttaaaggatgatatctactcggccgaatggagagccgtatccgcagacggtcatcctgtatcaggcacgatcccttttagtatcggaaacattcaaggcgggctgccgaaagatcagcaaaatcaagcatcaggcctgccggcagccgatatcgtcattgaacgcgtccttttgtataccggtttctccctgttcatgggagccgctctttttcagctcgtctggcaccgacctgaaaacggctggccgggtgacaaaaagcgccgtctgaagcggatcacgaatgtggcgctcttattgacaggggctgccatcatcattcagctgccactgcaaacaaaaacaaacgcaggcgtttcctggatcggggcttttcagccttcgcttttaaaagaaacgcttctgaatacgacgggcggaacgctgtggctgatcaacatcgccatcatcgccttgcttgttttctcttcgaatagaaaaggcctgaagagcgcgatcttgagcctgttcctttttgccggactattgttcgtcaaatcgctcgacggccatgccgcttcgacgagcgcaggctatatgacaatcgcgatgaacttcatccatctgaatgccgcttccatttggactggcggactcgcgatgtttgtgctgtttttcggaaaagactggctgaagcctgacaaaagtttgatatgggaaaccattcggcgcttctcgccatgggcgctcgcttcagcagggctactcatattctcgggccttttcaacagctttttcattgttcatacactggataacctgttcgggacggcttacggaaaagcgctccttgtgaaatcggcgctcgtccttctcatgatcggactcggggcatttcactattttcgttttagaaaagcgccgaaacagcagggcggccgttcgatgaaagctgaatgggcggtcgggcttctcattctgctgaccacggcggttttcaccaatattccaagtccgcccgcgccgctgccgcagcctttcagtgagacgaagcagctgacggaaggaaatacgctgtcactccgaatcagcccaaacatgccgggctcaaatacgtttgaagtcaccgtcagaaacagaaaaggagacgtcgtcaaagatatccaaaaaattgaattaaccgttgagaaaaccgggcttttcggggatgacaaggaaagcacgttcagcctcagcaaaaataaagacggtgttttccaaacgaaaaatttaaatttaagcgaagacggcatctggaagatccgcgttcacggattaacggcgtcatttgaggaaatcagaacagttttcaatgcaaaaattaataaaaaggagttttga

SEQ ID NO: 30 - protein, 548 aa, Bacillus licheniformis DSM 13
MNVMKRATLWAVLAVLCTILLPKAAFAHAYIVQSTPAENQELKKAPEEIKIEFNEAIQNGFHSITVRNSSGERVDSGKTEIDPQNAKIMTVKLKPKLKDDIYSAEWRAVSADGHPVSGTIPFSIGNIQGGLPKDQQNQASGLPAADIVIERVLLYTGFSLFMGAALFQLVWHRPENGWPGDKKRRLKRITNVALLLTGAAIIIQLPLQTKTNAGVSWIGAFQPSLLKETLLNTTGGTLWLINIAIIALLVFSSNRKGLKSAILSLFLFAGLLFVKSLDGHAASTSAGYMTIAMNFIHLNAASIWTGGLAMFVLFFGKDWLKPDKSLIWETIRRFSPWALASAGLLIFSGLFNSFFIVHTLDNLFGTAYGKALLVKSALVLLMIGLGAFHYFRFRKAPKQQGGRSMKAEWAVGLLILLTTAVFTNIPSPPAPLPQPFSETKQLTEGNTLSLRISPNMPGSNTFEVTVRNRKGDVVKDIQKIELTVEKTGLFGDDKESTFSLSKNKDGVFQTKNLNLSEDGIWKIRVHGLTASFEEIRTVFNAKINKKEF

SEQ ID NO: 31 - DNA, 573 bp, Bacillus licheniformis DSM 13
  gene (1)..(573): ycnK - unknown function - similar to transcriptional regulator (DeoR family) - BLi00481
atgctaccgattaaaagaagggagcagattttggcttggatcaaagaagaagaatctctgcgcatttcggaaatttcgaaaaggctgaacgtctctgaaatgaccgtttaccgcgacattaaaccgctcgttgaaaacgggcaggtcatcaagaccgcaggcggaattacgctaaacaagccgaagcagcagcttgggcagctttgcgtcgtttgcggaaaaggggcaggttcgaggcttgccgtgcaaattgtcaaaaacgacagccaaattgaacagttttgctgcgtgcattgcgcaatgctccgctatgagaaagtgaaagatgatgtatcacagattatctgcagggattttttgctggatacgacgatcagcgccaaagctgccgtctttttgctggatacagatcttcatttgaactgctgcgagccgcatgcgctccctttcgcttctgaagcagaagcgcggaaattcaaaaaaggctttggcggacagctgttatcgtttgacgatgccgcgcttctggtccagaaaacgatgaagaacagctgctgcagcctgaagacatga

SEQ ID NO: 32 - protein, 190 aa, Bacillus licheniformis DSM 13
MLPIKRREQILAWIKEEESLRISEISKRLNVSEMTVYRDIKPLVENGQVIKTAGGITLNKPKQQLGQLCVVCGKGAGSRLAVQIVKNDSQIEQFCCVHCAMLRYEKVKDDVSQIICRDFLLDTTISAKAAVFLLDTDLHLNCCEPHALPFASEAEARKFKKGFGGQLLSFDDAALLVQKTMKNSCCSLKT

SEQ ID NO: 33 - DNA, 2295 bp, Bacillus licheniformis DSM 13
  gene (1)..(2295): nasB - assimilatory nitrate reductase (electron transfer subunit) - BLi00482
atggggaaacaaaggcttgttttaataggaaacggcatggcaggggttcgcacggccgaagagatttcactacacggcgggagccggtttgagatcgtcatcatcggcagcgaaccacacccgggctataaccggattctgctgtcttctgtccttcagggagaaacggattggaacgatgtgatgacaaaaagccggagctggtatgaagaaaacgggattaccctttatacaggggaaacggctgtcgcaatcgatacggttaatcaaacggtggcgacagatcaaaaccgcgaaatcgcttatgacaaactgatcattgcgacgggctcttccccgtttatcctcccggttcatggtgcagacaaagaaggggtatatggttttagaacaatagacgattgccgggcattcataaacgcatcaaagcgatttaaaaaagcggccgtcatcggcgggggaatattgggccttgaggcggcgcggggattggcaaacctcgggatgaaggttgatgtggttcaccactcttcctccatcatgcagactcagcttgatccgccggcttctgccatgctccagaaagagctggagcgtcaagggatccattttcttttggagaaagatacagaggcgattttaggaacatcgcgcgccgagggtgtgcggtttaaagacgggacgaagatcagcgccgatctcgtcgtcatggctgccggtgtcagaccgaatatcgaactggccaaggcgagcggtatttcaaccaacagggcgatcatcgtcagcgactatatggagacaaatgttccaaatgtctacgccgtcggtgaatgcgcggagcataacggaacggtctacggcttagtcaagccactgtatgagcaggggaaagtgctggccaaacatatttgcggcctcgaatgttccggttatcagggctccgttcagtccgcggctttaaaaatgtccggaatcgacgtcttttcggcagggaaaatcacagaggacgactcgacgacggctattaaacttttagacgaagcggccggagtctataaaaaagcggtctttcaaggggataaaatggccggcgtcattttatacggagacaccagcggcagccaaaggctgttggaaagcatcatcaaacaaagggatattaccgttgtcaaaaaagagctgtttcaatcggaggaagacagttcagtcgcttctatggcggttcgtgaaacgatctgccaatgcaacgcggtttcaaaaggcatgatcatggaagcgatacagacgcacggtttaaagacggccgaggaggtcaaacgctgtacaaaagcttccgggtcatgcggcggctgcaggccgttggtcgaggaactgctcctccataccgccgaacatgatggtcacatatctgcggcggaacaaccgatgtgcgcctgcacttcattcacggaagatgaagtcgtgaacgaaatacagatgagaaatctttcttccgttcacgaagtcatctcggcgctcggctggaaaaatagcagcggatgccggatttgcgtgcccgcgcttcactactatttaaaaatgatccgtccggacatcgcctatgaggagcacgaagaggagacggatgccatcatcccgcaaatgtacggaggccggacgaatgccgaagagctgaagcgcattgcaggagtgattgaaaaatatcaaattcaagaggtgtacatgacacaccatcagagattaaagcttgccggcatcaagccggagcatatagagcgcgtgaaagaagaactcggcatgccgtattgcctgccgcaacagcagcggatcgccgcggttaaaacgtgcccatgcggatcaccgcagattcaaacattggcagcagacttggaaaaagcagcagaatccctcctcattccggccaaggtctccattggggtgtcgggctgtttggacgattgcgtgtacgcttcagttcatgacatcggacttctcaaagtaaacggaggatgggagatttatgcgggcggccaaggaggacagcatgcttccccgggagagctgctttcagtagccgactcggcggaggaagcgggtgaactcatgaaaggcctgcttcaatattaccgcgaaacagcaaactatcttgagaagatcgggcattggattgaacgcgccggcatcattcatatccgggaagttttatttgacaacggacttcgcggccagctgttcgaacggcttgaaaaagaaaagcggctggctcagcaagaaatgataaaagggctgatgtga

SEQ ID NO: 34 - protein, 764 aa, Bacillus licheniformis DSM 13
MGKQRLVLIGNGMAGVRTAEEISLHGGSRFEIVIIGSEPHPGYNRILLSSVLQGETDWNDVMTKSRSWYEENGITLYTGETAVAIDTVNQTVATDQNREIAYDKLIIATGSSPFILPVHGADKEGVYGFRTIDDCRAFINASKRFKKAAVIGGGILGLEAARGLANLGMKVDVVHHSSSIMQTQLDPPASAMLQKELERQGIHFLLEKDTEAILGTSRAEGVRFKDGTKISADLVVMAAGVRPNIELAKASGISTNRAIIVSDYMETNVPNVYAVGECAEHNGTVYGLVKPLYEQGKVLAKHICGLECSGYQGSVQSAALKMSGIDVFSAGKITEDDSTTAIKLLDEAAGVYKKAVFQGDKMAGVILYGDTSGSQRLLESIIKQRDITVVKKELFQSEEDSSVASMAVRETICQCNAVSKGMIMEAIQTHGLKTAEEVKRCTKASGSCGGCRPLVEELLLHTAEHDGHISAAEQPMCACTSFTEDEVVNEIQMRNLSSVHEVISALGWKNSSGCRICVPALHYYLKMIRPDIAYEEHEEETDAIIPQMYGGRTNAEELKRIAGVIEKYQIQEVYMTHHQRLKLAGIKPEHIERVKEELGMPYCLPQQQRIAAVKTCPCGSPQIQTLAADLEKAAESLLIPAKVSIGVSGCLDDCVYASVHDIGLLKVNGGWEIYAGGQGGQHASPGELLSVADSAEEAGELMKGLLQYYRETANYLEKIGHWIERAGIIHIREVLFDNGLRGQLFERLEKEKRLAQQEMIKGLM

SEQ ID NO: 35 - DNA, 2160 bp, Bacillus licheniformis DSM 13
  gene (1)..(2160): nasC - assimilatory nitrate reductase (catalytic subunit) - BLi00483
atgacagaacgattgctaaagtactttcgcacaaagcaaaaagaagtgcaatccgaaaaaacgtatgacacccaatgtccattttgcagcatgcagtgcaaaatgcagctcattgaacagacgattgtgacaagaaaaaaatacacggcggtcggaaaagacaatccgacgacaaaggggcggctgtgcatgaaagggatgaacgcccatcagcacgcgtttcattcagagagaatcacccatccgctgatcaaaaaaaacggcgaatttgtccgtgtatcatgggaagaagcgcttcagtacatcaaagaacaagtaacagcgatccagcatgaagacggccgcgatgctttgagtgtatacggcagcgcgtccattacaaacgaagaagcgtacctgctcgggaagttcgcccgcgtcgccttgaagacgaagcatattgactacaacggcaggctttgcatgtcggcggccgcaacagctgcaaatcaaaccttcggaatggaccggggactgacaaatgccctttccgagatcccccatgcaagggtcatcatcctcgcaggtacgaatatcgccgagtgccagccgacgatcatgccgtattttgaagaagcgaagcaaaacagggcgtacatcatcgtcattgatccccgcgaaacggcaacggccaagatcgccgacctgcatttgaaaatcaagccgggaacagacgccgcgctcgcaaacggaatgctgaaaatcatcattgaagaacagctgacagacggagcttttatcagagatagagctgaagggtttgaagaggtcaagcgccatgtagcatctttatcgcttgaagacatttcggccaaaaccgggatacaaatcgagcagatcaaaaaagccgcggtaaaattcgcccgcgaggagacgggaatgctgttcacggcaagaggggttgaacagcagactgacggaacggctgctgtaagaaatttactgaatattctaatatcaacggggaaaatcggcaaatatgcttgcggctacggcgccgtcaccgggcaaggaaacgggcagggggcgcgggagcacgggcagaaagcagaccagctgccaggttaccgttcgattgaaaatgaagccgaccgcgcctatattgcggatgtttggggcataaaagctgaagatcttccgggaaaaggcgtatccgcttatgaaatgatgaagaaagttcatgaaggcgaaattacaggaatgtttttaatgtgttcaaaccctgttgtatccagcccgaccgccggctttgtcaaatccgctttgggtaaattaaagttttttgccgccgttgatttgtttctttctgaaacggcaatactcgcggatgtcgttctcccagcatcatcctatctcgaggacgaaggaacgatgaccaatctggaaggcagagtgacattaagggaagcctcaaagccgtgtcccggtgaagcgaggcatgactggcaaattatttgcgatatcgctaaagcgcttggaaaaggccgctatttttcatatcgatcggcagaggatgtttttaatgaattgagaaaagcgagccgcggcggaattgccgactattcgggcatcacctatgaacgtctgagaaaggaaggcggcattttatggccctgtccagaagaggggcaccctggaacacagcgcctgtttgaatcatccttctcccacccggacaaaaaagcaagaatgtgcgtcgtcccgaacgaaccaaaggtcaaaaaagatgtaccgactgaggagtatccccttattctgacgaccggaagggtgatgtctcactatttaaccggggtacagacgagaaggagcgctgcgctcaaggcgagacactttgaatcatttatggaaatccatccgaaaacggcggaaaagtacaatattggcgaccgggtgctggtcagggtggaatcaaaaagaggcagcatcatcgtcaggagcaaatggtcggaagccatcaggccggataccgtttttgtaccgatacattgggcggattcgcaaaatgtgaacgatttgatagcggaaacgctcgatccgtcctgcagcatgccgggatttaaggtttgctccgtgaaaattcgtccgtcagttgacaattttgtgaataaattgtcataa

SEQ ID NO: 36 - protein, 719 aa, Bacillus licheniformis DSM 13
MTERLLKYFRTKQKEVQSEKTYDTQCPFCSMQCKMQLIEQTIVTRKKYTAVGKDNPTTKGRLCMKGMNAHQHAFHSERITHPLIKKNGEFVRVSWEEALQYIKEQVTAIQHEDGRDALSVYGSASITNEEAYLLGKFARVALKTKHIDYNGRLCMSAAATAANQTFGMDRGLTNALSEIPHARVIILAGTNIAECQPTIMPYFEEAKQNRAYIIVIDPRETATAKIADLHLKIKPGTDAALANGMLKIIIEEQLTDGAFIRDRAEGFEEVKRHVASLSLEDISAKTGIQIEQIKKAAVKFAREETGMLFTARGVEQQTDGTAAVRNLLNILISTGKIGKYACGYGAVTGQGNGQGAREHGQKADQLPGYRSIENEADRAYIADVWGIKAEDLPGKGVSAYEMMKKVHEGEITGMFLMCSNPVVSSPTAGFVKSALGKLKFFAAVDLFLSETAILADVVLPASSYLEDEGTMTNLEGRVTLREASKPCPGEARHDWQIICDIAKALGKGRYFSYRSAEDVFNELRKASRGGIADYSGITYERLRKEGGILWPCPEEGHPGTQRLFESSFSHPDKKARMCVVPNEPKVKKDVPTEEYPLILTTGRVMSHYLTGVQTRRSAALKARHFESFMEIHPKTAEKYNIGDRVLVRVESKRGSIIVRSKWSEAIRPDTVFVPIHWADSQNVNDLIAETLDPSCSMPGFKVCSVKIRPSVDNFVNKLS

SEQ ID NO: 37 - DNA, 2421 bp, Bacillus licheniformis DSM 13
  gene (1)..(2421): nasD - assimilatory nitrite reductase (subunit) - BLi00484
gtgaaaaaaaagcagcttgtccttgtagggaacggtatggccggggtcagggccattgaagaaattcttaaaatttcaaacgaagagtttcagatcacgattttcggcagcgaaccgcacccaaactataaccggattcttctttccaaagtgcttcaaggagatacggatgttaaggacattacactgaatgattgggattggtatgaagaaaacggcattcagctgtatacaggtgaagaagtcgttaaagttgatccggagtccaaaaccgtcatcacggatacgggaagagtccagccgtatgatgaactgattttggcgacaggttctctcccttttattctgccattgcccggagcggataaagaaggtgtaacagctttccgtgatatcaaggatacggatatcatgctcgaagcttccaaaacgtacaaaaaagcagcagttatcggcggcgggctgctgggactggaagcagcgcgcggacttctcaaccttggcatggatgttaccgttgtccacctcgcgccatatttgatggagcgtcagcttgatgcagccgcaggacggcttttgcaaaaagagctggaaaagcagggcatgaaatttttactggaaaagcagaccgaagaaatttacggagaaacacgtgtcgaaggcttaaaattcaaagacggctcaacgcttgaggccgatttggtcgtcatggcggtcggaatcaagccgaacgttcagctcggcaaagactgcggccttccggtaaacagaggaatcgtcgtcaatgattacatggagaccgaagttcccaacatttacgctgtcggagaatgcgcggagcataggggcattgcctacggacttgtcgcgcctctttatgaacaggcaaaagtgcttgccaaaaaaatctgcggcgttgaaacggctccgtatgaaggatctgttttatctacccagctgaaggtttccggtgttgaggtcttttctgccggtgactttaatgaggatgacggagaagacaaaaaagcgctcaaagtgtttgacgaacaggacggaatctacaaaaaaatcgttctgcgcggcaatcaggtgatcggcgccgtactgttcggagacagcagcgaaggaaacaggctgttttccatgattcaaaaaggagcggatatttctgatacttcaaaaatttcaatcttgcagcctttgaatcaggaagaaggcatgagcttgaccgctgcgatgagcgacgatgagatcgtgtgcggctgcaacggcgtttcgaaaggaatgatcattcaggcgatcaaagagcagggatgcacatcgacggatgaaattaaagcatgcacaggcgcatcgcgctcttgcggaggatgtaagccgcttgtcgcagagattctccagcatacgctcggctcagaatttgatgcttcagcacaaaaagaagcgatttgcggatgtacggacctttcaagagacgaactcgtggccgaaatcagagaaaaagggctgacccatacaaaagaagtcatgaacgtgctcggctggaaaacggcggaaggctgttccaaatgccgccctgcgcttaactactacctcggcatgatcaatccgaaggaatatgaagatgaaagagaatcccgcttcgtcaatgaacggatgcacgccaacattcaaaaagacggcacatattcagtcgtaccgcggatgtacggcggggtcaccaattcgaatgacttgcggcgcatcgccgatgtcgtcgacaaatatgaaattcctctagtgaaaatgaccggcggccagcggatcgatctgatcggcgtcaaaaaagaggatcttccgaaagtatgggctgatcttgacatgccttctggatttgcttacggaaaaacgctcagaaccgtaaaaacgtgcgtaggcgaacagttctgccgcttcggaacgcaagattcaatggctctcggaattgaccttgagaaaaaatttgaagggctctacacgcctcacaaaattaaaatggcggtttccgcttgtccgagaaactgtgctgaatccggcattaaagatatcggcgttgtcggcatcgacggcggctgggaaatttacgtcggcggaaacggaggcacacatctgcgtgcaggcgatctgttaatgaaagtcaaaacggctgaagaagtcatcgaaatcacaggcgcatatctccaatactatcgtgaaacagccaactatttagagcggacatctgcatggatcgaacgtgtcggcttgtcccacgtccaatccgtattgaatgataaagaaaaacgcaaagagctgaatgaccgcatcaatcaagcgctgtctgtccataaagacccgtggaaagactttttggaagacggaaagacaacaagacagctattcgaaaatgttgtcacttcctaa

SEQ ID NO: 38 - protein, 806 aa, Bacillus licheniformis DSM 13
  misc_feature (1)..(1): Xaa can be Val or Met.
XKKKQLVLVGNGMAGVRAIEEILKISNEEFQITIFGSEPHPNYNRILLSKVLQGDTDVKDITLNDWDWYEENGIQLYTGEEVVKVDPESKTVITDTGRVQPYDELILATGSLPFILPLPGADKEGVTAFRDIKDTDIMLEASKTYKKAAVIGGGLLGLEAARGLLNLGMDVTVVHLAPYLMERQLDAAAGRLLQKELEKQGMKFLLEKQTEEIYGETRVEGLKFKDGSTLEADLVVMAVGIKPNVQLGKDCGLPVNRGIVVNDYMETEVPNIYAVGECAEHRGIAYGLVAPLYEQAKVLAKKICGVETAPYEGSVLSTQLKVSGVEVFSAGDFNEDDGEDKKALKVFDEQDGIYKKIVLRGNQVIGAVLFGDSSEGNRLFSMIQKGADISDTSKISILQPLNQEEGMSLTAAMSDDEIVCGCNGVSKGMIIQAIKEQGCTSTDEIKACTGASRSCGGCKPLVAEILQHTLGSEFDASAQKEAICGCTDLSRDELVAEIREKGLTHTKEVMNVLGWKTAEGCSKCRPALNYYLGMINPKEYEDERESRFVNERMHANIQKDGTYSVVPRMYGGVTNSNDLRRIADVVDKYEIPLVKMTGGQRIDLIGVKKEDLPKVWADLDMPSGFAYGKTLRTVKTCVGEQFCRFGTQDSMALGIDLEKKFEGLYTPHKIKMAVSACPRNCAESGIKDIGVVGIDGGWEIYVGGNGGTHLRAGDLLMKVKTAEEVIEITGAYLQYYRETANYLERTSAWIERVGLSHVQSVLNDKEKRKELNDRINQALSVHKDPWKDFLEDGKTTRQLFENVVTS

SEQ ID NO: 39 - DNA, 1461 bp, Bacillus licheniformis DSM 13
  gene (1)..(1461): nasF - uroporphyrin-III C-methyltransferase - BLi00486
atggggaatgggaaagtttattttgtcggggccggaccgggcgattcaggattgatcacattaaaaggaagaacgctgatcgaggcggcggatgtcatattgtatgacaggctcgtcaattcccggctgcttgagcatgcaaaagcatcatgcgaatttgtctactgcggcaagctcccggaccgccattatatgaggcaaagtgaaatcaatgcactgctcattgaaaaggggctgaaaggcctcaaagtggtccggctgaaaggaggagacccgagcgtgtttggcagagtcggggaagaagcggccgccgtcagcgaacacggcatcccgtacgagatggtgccaggcattacatcgggaatcgccgctcctctttatgccggactgcctgtgacccaccgcgatttcgcttcatcctttgccgttgtcaccgcccatgataaatcgggcaaaccggagatgaattgggaagggctggccaagggcgtccaaaccctcgtcttttacatggggatcaaaaaccttggttatatatgcgagcagctgatgaaacacggcaagtccccccttgttcccgcaatggccatccaatggggcacatggggccggcagcggagtgttaaagggacattgcagaatatcagggagaaaataaccgagcatcagatcaaaaatcccgcgattatcgtcatcggcgacatcgtatcattcaggcagaacagctggtttgaagaaaagcctttgatcgggctgaacatcatggcagtcaaaagctgtgaagaagaacaaattccagtggaggcgcttcgtgaacaaggcgctgaagttgtggaatggccgaaatggaaatcggccgaaacggtgcctgacgatgcacttttaggtcgaatcgcttgttttgacagcctgctgttcacctcaagtgcgtcactgcgcagatttttcagccggcttgctgctttgaagatcgatgtcagggaaatgcgcggaaggctttacgcttcagatcagcatgtaaaagcagctcttgaagaacggggatttgaagcctttctgcaaacggaaatgccggtctcgggcagctgtttgctgatcggcagccgggaagccgtgcaaaattacccgcactccgggtgcgaggtattgattacacatgacagggtgatcgacagccgctttacctcgatcatcaaacgggaaattgatgagtttcctattgatatagtgttgttttcaaatcgaacatgcgtcgagctgttcgtaaacgaggcggagacagtcggcctccatccggaaaaaacggctgcccgtcttcaggcgttctgcctcactgaagaagcgggggagagagctgcagccgtagggtttcagcgcgttcatgcagtgaatgatcgcgatcgcctgccggatgtcatcaaagagatcaaccgaacagccgaatttgtctgtcagagctga

SEQ ID NO: 40 - protein, 486 aa, Bacillus licheniformis DSM 13
MGNGKVYFVGAGPGDSGLITLKGRTLIEAADVILYDRLVNSRLLEHAKASCEFVYCGKLPDRHYMRQSEINALLIEKGLKGLKVVRLKGGDPSVFGRVGEEAAAVSEHGIPYEMVPGITSGIAAPLYAGLPVTHRDFASSFAVVTAHDKSGKPEMNWEGLAKGVQTLVFYMGIKNLGYICEQLMKHGKSPLVPAMAIQWGTWGRQRSVKGTLQNIREKITEHQIKNPAIIVIGDIVSFRQNSWFEEKPLIGLNIMAVKSCEEEQIPVEALREQGAEVVEWPKWKSAETVPDDALLGRIACFDSLLFTSSASLRRFFSRLAALKIDVREMRGRLYASDQHVKAALEERGFEAFLQTEMPVSGSCLLIGSREAVQNYPHSGCEVLITHDRVIDSRFTSIIKREIDEFPIDIVLFSNRTCVELFVNEAETVGLHPEKTAARLQAFCLTEEAGERAAAVGFQRVHAVNDRDRLPDVIKEINRTAEFVCQS

SEQ ID NO: 41 - DNA, 1191 bp, Bacillus licheniformis DSM 13
  gene (1)..(1191): hypothetical protein - unknown function - BLi00236
atgaaaagtctaaacaacattaaaaataaaattgtcatagttggaacagttgatagcctaggtgcagccattgtatggacagggttaccactttatgcatattcgattacgggatcttttttatatacatcatcattattttttacttcagcaatagcccgtatattagcaaccttttttggaggctatttagcagatgtatcatcgagtaaaagaataatgctaagatgtttaagtattaattggtttttttattcactattatttttgggattatcactcggtttaacttggattatattccctatcatgttgatttcacaatgtttcagtaccatatcgatcatgaatcaaaacctttggtttaattcactttgctcaaaagagaccctggcttccgaaataagtggtagaaactcatggtttttgattagtaaaacagttggtttctcattaggaccactcatgtatcattggcttaatgcatactcattaattatcaatctattaactatattgattgcaataacattaatattaacaataaaagatttaggttatgtaaataataaaaagaaaaaagagagcattttttcttcgttcaaagaagtttataaaaatcaattattaaaaaggctaaatataatagaaattttaaatggtttaacttttcctgttttaacaagcttatcgatatatattcttgaaaagaatttaaatgcttcttctcttataatttctttgttttggttgataggaggagtaggagcgattacggctaacttgatgctttcgagacttaaattatttaaattacagtatattcatcaatttataataagttttgttctcgtagttggtggattgttgttaatgacgatagctagctattcttttgtctatttaataggattttctttatttactttaggtacccctataataaataatttattaaggtcaaaagtgtttattttcgccccaagtcatatgaaaggaaaagtaacatcagttataacgtcttcatcagatttaggcactattatagccttatcattgtcttggattgttgttgaaaaattcggggtattaatttttatgtcttttgttattttaacaagtgtaagtcgaatatgcctttttttctacactctacataaagtagaaaataaagttgatgtggagggataa

SEQ ID NO: 42 - protein, 396 aa, Bacillus licheniformis DSM 13
MKSLNNIKNKIVIVGTVDSLGAAIVWTGLPLYAYSITGSFLYTSSLFFTSAIARILATFFGGYLADVSSSKRIMLRCLSINWFFYSLLFLGLSLGLTWIIFPIMLISQCFSTISIMNQNLWFNSLCSKETLASEISGRNSWFLISKTVGFSLGPLMYHWLNAYSLIINLLTILIAITLILTIKDLGYVNNKKKKESIFSSFKEVYKNQLLKRLNIIEILNGLTFPVLTSLSIYILEKNLNASSLIISLFWLIGGVGAITANLMLSRLKLFKLQYIHQFIISFVLVVGGLLLMTIASYSFVYLIGFSLFTLGTPIINNLLRSKVFIFAPSHMKGKVTSVITSSSDLGTIIALSLSWIVVEKFGVLIFMSFVILTSVSRICLFFYTLHKVENKVDVEG

SEQ ID NO: 43 - DNA, 1584 bp, Bacillus licheniformis DSM 13
  gene (1)..(1584): pckA - phosphoenolpyruvate carboxykinase - BLi03197
atgaattctgtagatttaacaactgacttacaaacgctattaacgggcgaaaacgttcagatgaacttatccgttccccatttggttgaaaagatccttgagcgcaaagaaggagtcttgacatcttccggagccgtgcgggcaacaaccggtacatacaccggccgctcgccaaaagacaaattcattgtggaagaagcgagcacaaaggacaaaatcgattggggcgcagtaaaccagccgatttcagaagccgcctttgaccggctgtacgcaaaagtgctcggctatttaaaggaacgcgatgaattgtatgtatttgaagggtttgcaggagcagacgaaaaataccggcttccgattactgttgtcaacgagttcgcctggcacaacctgtttgcaagacagttgttcatcagacctgaagaacatgataaaaaacctgaagaacagccgtttacgattctttcagctccgcatttcaaagccgatccgctcgttgacggaaccagatctgaaacgtttatcatcgtgtcgtttgagaagcggacgatcttaatcggaggcaccgaatatgccggagagatgaaaaaatcgattttttctattatgaattttcttctgcctgaacaaaacattctaccgatgcactgttcggcaaacattggcgaggaaggcggaactgctctcttcttcggattgtcaggtacaggcaaaacgacgctttccgccgatccgaagcggaagctgatcggcgacgacgaacacggctggtccagcacaggcgttttcaacattgaaggaggctgctacgccaaatgcattaacttaagcgaagaaaaagaaccgcagatttacaatgcgatccgcttcggttccgttctcgaaaatgtcgtggttgacagcaaaaccggagagccggactattccgactcattctacacggaaaacactagagcggcttacccgattgacgcgattgacaacatcgtcaagccgagcatcgcagcgcacccgcagaccatcgtatttttgaccgcagatgctttcggcgtgcttcctccgatcagcaagttaacgaaagaacaagccatgtatcatttcctgagcggatatacaagcaagctcgccggcaccgaacgcgggatcaccactcctgaagccacattctcgacctgcttcggttcaccgttcctcccgcttccggcacatgtgtacgctgaaatgctcggaagaaaaatcgatgagcacggggtcggcgtctttctcgtcaatacaggctggaccggcggcggatacggaaccggggaaagaatgagcctcgcatatacgagagcgatggttcaggccgcaatagaaggagaactggataaagccgaaatgaggactgaccgcattttcggacttcattcacctgtccacgttccgggtgtgcctgatcaagtgcttgacccggccaaaacttggacagatgaaaacgaatacgaggaaaaagcgatccacctggcgaaagcattcaaacagaatttcaaaaagttctccaatacggaaaacatcgaaaaagccggaggaccgctcgtttaa

SEQ ID NO: 44 - protein, 527 aa, Bacillus licheniformis DSM 13
MNSVDLTTDLQTLLTGENVQMNLSVPHLVEKILERKEGVLTSSGAVRATTGTYTGRSPKDKFIVEEASTKDKIDWGAVNQPISEAAFDRLYAKVLGYLKERDELYVFEGFAGADEKYRLPITVVNEFAWHNLFARQLFIRPEEHDKKPEEQPFTILSAPHFKADPLVDGTRSETFIIVSFEKRTILIGGTEYAGEMKKSIFSIMNFLLPEQNILPMHCSANIGEEGGTALFFGLSGTGKTTLSADPKRKLIGDDEHGWSSTGVFNIEGGCYAKCINLSEEKEPQIYNAIRFGSVLENVVVDSKTGEPDYSDSFYTENTRAAYPIDAIDNIVKPSIAAHPQTIVFLTADAFGVLPPISKLTKEQAMYHFLSGYTSKLAGTERGITTPEATFSTCFGSPFLPLPAHVYAEMLGRKIDEHGVGVFLVNTGWTGGGYGTGERMSLAYTRAMVQAAIEGELDKAEMRTDRIFGLHSPVHVPGVPDQVLDPAKTWTDENEYEEKAIHLAKAFKQNFKKFSNTENIEKAGGPLV

SEQ ID NO: 45 - DNA, 1590 bp, Bacillus licheniformis DSM 13
  gene (1)..(1590): putative protein - putative malate synthase (EC 4.1.3.2) - BLi04208
atgaaaacagaggagaagagaattcatataaccggatcgataaatccgcattatgaagagattttgacaaaagaagcgatggagtttatcggacgtcttgaacgccatttcgggggaagaagacgggaattgctgcatttgcggacgatcaggcaggctgaaattgatcaagggaagaacccttgttttttggcggaaacgaagcacatccgcgaggcggactggacagtcgcgccgctcccccgggatttggaggaccgcagggttgaaatcactggcccggtcgacagaaaaatggtcatcaacgctttaaattcaggagcgaaagtctttatggccgactttgaagatgcgaactcgccgacgtggaaaaactgcattgaaggccaggttaatttgcgggatgcggtccgcgggacgatttcttttacgaatgaacagggcaaacgctatgagctgaaagagaaccccgccgttatcatggtcaggccgaggggatggcacttggaagaaaagcatgtgctgatcggcggcaggcccgtgtcagcaagcctgttcgattttggtctttattttttccataacgcaaaagagctgatcaaaaaaggaagcggcccttatttctaccttccaaaaatcgaaaaccacctggaagcaaggctgtggaacgacgtatttgtttatgcgcaaaacgacttgcgcattccgcagggcacgattaaagcgacggttctcgttgaaacgatcctggcggcctttgaaatggatgaaatcctctacgaattgaaggaacattcagccgggcttaactgcggcaggtgggattatattttcagctttattaagaaattcaggaacagggatgaagtcattcttccagatcgctcagccgtgacgatgacagttcccaacatgagagcctactccctcttggcgattcaaacgtgtcataaacgcaacgtccatgcgattgggggaatggcggcgcaaattcccgctaaagacaaacctgagatcaatgaagccgcatttgccaaaatccgcgaagataaggaacgtgaagtaaaagacggccatgacggaacatggatcgcgcatcccggcatgattcaaacggcgctcgatgtattcaatcagcatatgccgtcggcgaaccagattcatagaaagcggaaagacgtaaaggtcgaggaaaaagacttgctggaagtaccgaaaggcagaattacagaagcaggcgtccgtaccaatatcagcgccggcatccgctatttagcttcatggctgtcgggcagaggcgccgctccgatcgaccacttaatggaggacgctgcaacggcggagatttccagagcccagctgtggcaatggattcgccatccgaaaggagttcttgacgatggccggaaagtcaccgcggaaatggtcaagatgatgaaagagaaagaaatgcagaaaatcatcgatgaagcaggcgtggaacattttcggagcggccggtttgaagaggccggacagttatttgaacagctgattttggatgatgaattcgcggagtttctaacgataccggcttatgaaaatatataa

SEQ ID NO: 46 - protein, 529 aa, Bacillus licheniformis DSM 13
MKTEEKRIHITGSINPHYEEILTKEAMEFIGRLERHFGGRRRELLHLRTIRQAEIDQGKNPCFLAETKHIREADWTVAPLPRDLEDRRVEITGPVDRKMVINALNSGAKVFMADFEDANSPTWKNCIEGQVNLRDAVRGTISFTNEQGKRYELKENPAVIMVRPRGWHLEEKHVLIGGRPVSASLFDFGLYFFHNAKELIKKGSGPYFYLPKIENHLEARLWNDVFVYAQNDLRIPQGTIKATVLVETILAAFEMDEILYELKEHSAGLNCGRWDYIFSFIKKFRNRDEVILPDRSAVTMTVPNMRAYSLLAIQTCHKRNVHAIGGMAAQIPAKDKPEINEAAFAKIREDKEREVKDGHDGTWIAHPGMIQTALDVFNQHMPSANQIHRKRKDVKVEEKDLLEVPKGRITEAGVRTNISAGIRYLASWLSGRGAAPIDHLMEDAATAEISRAQLWQWIRHPKGVLDDGRKVTAEMVKMMKEKEMQKIIDEAGVEHFRSGRFEEAGQLFEQLILDDEFAEFLTIPAYENI

SEQ ID NO: 47 - DNA, 1350 bp, Bacillus licheniformis DSM 13
  gene (1)..(1350): putative protein - putative hydrolase - BLi02233
atgcaaaaaacgctttttcaaaatgctacgattgtgacgcttgatccgcagcttcatacgctcgaaaaaggggacatgctgattgaaggctccgtcattctggacgttgccgagtcgattgaagccggagacgctgagattgtggatgcctccaatatgctgatcatgcctggatttgttgatacgcaccgccacgtatgggaatctgtgatccgcggaatcggagccgactggtccctgcaaacctatttaaaccggatttattatgggaattatgggtcgatgagaagacctgaggatgattgggccgcgaactatttaggagcgcttgaggcgctcgatgcaggcgtcacgactttgctggactggacgatgattgagtcgcctgatcatacggaccagctgattgccggactgaaagacgccggcattcgcgcagtattcgcctttggaacatctggtgacgccgaatattgggacagagacagcgatctttcgaatatggacgaagcgaaacgggtaaaaaaagcatattttcaatcgaaggatcagcttctgacgatggggcttgcaattcgaggtccggagttttcttcatggaagacatctgtctttgaaatcgaaacggcccgggaactggatgccttatgcagcatgcatattgcttttggcaactggggagcgaaagatcgttcgattgaaaagctgaatcaagccgggctgcttggtcccgatttaaacatggtgcatgccaatgcgatgagtgaagaagaaatgaaaatgcttgcggctcacggaagctcgatatctgtgacaccagaaattgagatgatgatggggcacggctatcctgttacagggctctctctaaaaaacggggttcttactgcgcttggggttgatgttgtcacatcgaccgggggagaccttttcgcacagatgaaatttgctcttcaagcagaacgcgcaaagcacaatgatgaactgctgagagaaggaattatgccggggcccgaattgagcatatcggccgaacaaatcctgaaagcggctaccattgacggggcgcgcgccctaaagctcgatcataaaatcggctcgctttccccaggcaaagaagctgactttatcatggtgaaaacggacagcgtcaatttgctccctttcactgatgcggcgggggccatcgtccaatgcacgcacaccggaaatgtagattccgtatatgtcgcaggaaaagcggtgaaacgaaacggccggctggttgatgcagatcttgaaaaggtgagacaaaaagcgctggaagcaagagatcatattttattccgatcccctgagggcgccttttaa

SEQ ID NO: 48 - protein, 449 aa, Bacillus licheniformis DSM 13
MQKTLFQNATIVTLDPQLHTLEKGDMLIEGSVILDVAESIEAGDAEIVDASNMLIMPGFVDTHRHVWESVIRGIGADWSLQTYLNRIYYGNYGSMRRPEDDWAANYLGALEALDAGVTTLLDWTMIESPDHTDQLIAGLKDAGIRAVFAFGTSGDAEYWDRDSDLSNMDEAKRVKKAYFQSKDQLLTMGLAIRGPEFSSWKTSVFEIETARELDALCSMHIAFGNWGAKDRSIEKLNQAGLLGPDLNMVHANAMSEEEMKMLAAHGSSISVTPEIEMMMGHGYPVTGLSLKNGVLTALGVDVVTSTGGDLFAQMKFALQAERAKHNDELLREGIMPGPELSISAEQILKAATIDGARALKLDHKIGSLSPGKEADFIMVKTDSVNLLPFTDAAGAIVQCTHTGNVDSVYVAGKAVKRNGRLVDADLEKVRQKALEARDHILFRSPEGAF

SEQ ID NO: 49 - DNA, 1359 bp, Bacillus licheniformis DSM 13
  gene (1)..(1359): putative protein - putative Na(+)-linked D-alanine glycine permease - BLi00817
atggaatttatttcacatattgtagatacaatgaacggttttttatgggggcctccgcttttgattctcattgttggaacagggatttatttgactttcaggctggcttttttgcaggtcagacttttgccttactctttaaagctcgccttttcaaggcatcaggataaatcctccgaaggcgatatttcccactttcaggcgctgatgaccgcccttgccgctacggtcggcacagggaatatcgtcggcgtcgcctcagctgttattgccggcgggccgggcgccgtattttggatgtggctcgccgccttcttcggaatggcgacgaaatatgctgaagcggtgcttgctgtcagattccgcgtgaaagataaaaacggcgaaatgtcaggcggtccgatgtattatttggaacacggcctcaagcaaaagtggcttggcgtgttatttgccatattcggggcgcttgccgcttttggaataggcaatatggttcagtcaaattctgtatccggagtcatgagctccactttttccgttcctgcctgggtaaccggcattattgtcacgacttttaccgcccttgtcattctcgggggaattaaaagcatcggcagagtcacctcgctgttcgtgccggtcatggctttattttatgtcatcgccggattgatcattcttgtcacaaactttgaacttgtccccggcgctgtcggtttaattttctctgacgcgtttaccgggcaagccgtggcaggaggcgcgatcggaaccgtgatccgctggggcgtagcgcggggcgtattttcaaatgaagcaggtctcggctcggcgccgatcgccgcagccgcggcaaaaaccgatatgccggcccgccaggcgcttgtctctatgacccaggttttcatcgacacattgattgtctgttcgattacggggatatcaattgtcatgggaaatatgtatctgacagaaaaaaccgatgccctgacatcgatgacattcgcacactttcttggtcctgcgggatcgacaatcgtcgcgatcggactgctgctgttcgcctactcgacgattttgggctggtcgtactacggagaaaaatgcttcacctacctgctgaatgaaaaatatgcgatcgtctatcgtatcatctttgtgatcgccgtctttttcggagcggtctttaaaaatgacttcgtctgggcgcttgccgatatgctgaacggcctgatggcaattccgaacctgatcggcctgctcggtttgacaggcattgtcgtctatgaatccaaacagatcctcgccaaaatcaaagccgaaaaagaagaaaagcaaaacagcgtatctgtttaa

SEQ ID NO: 50 - protein, 452 aa, Bacillus licheniformis DSM 13
MEFISHIVDTMNGFLWGPPLLILIVGTGIYLTFRLAFLQVRLLPYSLKLAFSRHQDKSSEGDISHFQALMTALAATVGTGNIVGVASAVIAGGPGAVFWMWLAAFFGMATKYAEAVLAVRFRVKDKNGEMSGGPMYYLEHGLKQKWLGVLFAIFGALAAFGIGNMVQSNSVSGVMSSTFSVPAWVTGIIVTTFTALVILGGIKSIGRVTSLFVPVMALFYVIAGLIILVTNFELVPGAVGLIFSDAFTGQAVAGGAIGTVIRWGVARGVFSNEAGLGSAPIAAAAAKTDMPARQALVSMTQVFIDTLIVCSITGISIVMGNMYLTEKTDALTSMTFAHFLGPAGSTIVAIGLLLFAYSTILGWSYYGEKCFTYLLNEKYAIVYRIIFVIAVFFGAVFKNDFVWALADMLNGLMAIPNLIGLLGLTGIVVYESKQILAKIKAEKEEKQNSVSV

SEQ ID NO: 51 - DNA, 1758 bp, Bacillus licheniformis DSM 13
  gene (1)..(1758): ggt - gamma-glutamyltranspeptidase - BLi01364
atgagacggttagctttcttagttgttgcgttttgcttggcggttggttgctttttcagtccggtgtcgaaagcagaaggagtcatgagcgggggagatggtgataaagtcgccgtcgggaaagacggtatggtggctacggcccatccgcttgcatcaaaaatcggagccgaagttttgaaaaagggcgggaatgccattgatgcagctattgcgatccagtacgcactgaatgtaacagagccgatgatgtctggaatcggcggcggcggtttcatgatggtctacgacggcgagacgaaggaaacgtcgatcatcaacagcagggagcgtgctccggagggagccaagcctgacatgtttttggatgaagacggaaaggtgattccgttttcagaaaggtcgagacacggaaatgcggtcggtgtgccgggaacgctgaaagggcttgaagcagcgcataagaagtggggaacaaagaaaatggaagacttgatttcgccttctataaagcttgcagaggaagggttcccaatcgactcggttttggctgacgcgataaaggatcatcaggacaagctgagcaaaacggccgcaaaggatattttccttccggacggtgaacctttgaaagaaggcgatatccttgtacaaaaggatttggccaaaacgtttaaattgatacggaaagaagggtcaaaagccttttatgatggggaaatcggccgggcgattgcagatgtcgtgcaggatttcgggggttcaatgacacctgatgacctgtcccgctacgaggtcacaacagataagcccatctggggcgagtaccacggctatgatattgcaagcatgcctcctccaagctcaggcggagtgttcatgctgcagatgctgaaattgattgatgattttcatttatcgcaatacgatccaaagtcgtttgaaaaatatcatctgctcgcggaaacgatgcatctctcctatgcggacagggctgcatatgcgggcgatccggaatttgtcgatgttccgctcagagggctgctcgatcccgattatattaaggagcggcaaaagctgatcagccttgattcgatgaaccgcgatgtgaaagagggcgatccgtggaaatatgaggaaggcgagccgaattatgagatcgtgcctcagcctgaagataaaacgatcggggagacgacgcattttaccgtcacggatcagtggggcaatgttgtctcttatacgacaacgattgaacagctttttggaacggggattttagtgcccgggtacggactgtttttgaacaatgaactgaccgattttgatgcgattccaggcggcgccaacgaagtgcagccgaataaaaggccgctttcaagcatgacgccgacgatcgtattcaaagatgaaaaaccggtgctgactgtcggttcccctggcggaacgacgatcattgcttccgttttccagacgatcttgaattattttgagtacggcatgagccttcaggatgcgattgaagagccgaggatctatacgaacagcctgacttcgtaccgctatgaatccggaatgccggaggatgtcagacggaagctcaatgacttcggccacaaattcggctcgaatccggttgatatcggcaatgtccaaagcattttcattgaccgggagaacaaaacgtttatgggggttgccgattcaagcagaaacggaacggcggttggggtcaacattaagacatcggctaaatag

SEQ ID NO: 52 - protein, 585 aa, Bacillus licheniformis DSM 13 [sequence omitted]

SEQ ID NO: 53 - DNA, 942 bp, Bacillus licheniformis DSM 13
  gene (1)..(942): hypothetical protein - unknown function - BLi04116
atgagggtcttgaaaaatgaactttacaggctgatggtgacgaaaagtacctggattgtgttaagcttgctgcttgtcatgacaatcgctgttgcatggatggtcagcaatggcgaaaaggagaaggagacaggtaactggaaagagcaattaaccgttcaaaacgctcagtatgaaagagaaatgagagagctgagcccagcggttcccaaataccaatttttaaaagaagagatcgcggtcaatcaataccggcttgagcataatttgccgccttctgcgaaatacaatgtttggacgatgctgaaggagttaaaaccgatcacaacattaatcgcgttgattgcaatcgtgctggcagcgaactccatcgcgcttgagcacagcaaaggaactattaaatttgcgattgcaacaccggtcaagcgttggcattatctattggggaaatacttgtcgatcttgctcaacactgtatttatgtttgctgcgacacttttatttgcgtttgttttagggtatgccctgctaggtttggagggaagccaatactatttgtcctatcgaagcggcgaagtgataaagatgtcaatgctgaagtttttagccttagactatggagccgctttattgaatattattgtgctggccacgctggcctttatgatttcggtcatcctaagaagcgcggtcgtctctgtcggtctttcgctgttcgtcttttttacagggtctgcgattactcaatttttagcggcaaaatttgattggaccaagtatacgatctttgcaaattcagatctcagtcaatacattgatggggagccgtttattcaagatatgactctttcattttcagcggctgtgattgctgtttattttattctttttctggctgtctccttttgggtttttcaaaagcgggatattgtcacgtcataa

SEQ ID NO: 54 - protein, 313 aa, Bacillus licheniformis DSM 13
MRVLKNELYRLMVTKSTWIVLSLLLVMTIAVAWMVSNGEKEKETGNWKEQLTVQNAQYEREMRELSPAVPKYQFLKEEIAVNQYRLEHNLPPSAKYNVWTMLKELKPITTLIALIAIVLAANSIALEHSKGTIKFAIATPVKRWHYLLGKYLSILLNTVFMFAATLLFAFVLGYALLGLEGSQYYLSYRSGEVIKMSMLKFLALDYGAALLNIIVLATLAFMISVILRSAVVSVGLSLFVFFTGSAITQFLAAKFDWTKYTIFANSDLSQYIDGEPFIQDMTLSFSAAVIAVYFILFLAVSFWVFQKRDIVTS

SEQ ID NO: 55 - DNA, 903 bp, Bacillus licheniformis DSM 13
  gene (1)..(903): putative protein - putative ABC transporter ATP-binding protein - BLi04117
atgagtcaagtcgaatttgaaggtgtaagtaaacgaataaaaggcagaccaattgtccaaaatatcacatttcaaattgccccaggtacaatttttgggctgctcgggccaaacggcgctggcaagacaacacttatcaaaatgattgtcgggatggcaaagccgacatcaggagatatccgcatcgacggctattcagttaaaagcaattacgaggaagcggcagcccgagtcggttctgttgttgaaaacccatccttttatgagcacttaacaggataccaaaaccttaaatatctcggcggattccacagccacgtgtcaaaggagcgcatagaagagatcgttcagcttgttgatttgacaggaagtattcataaaccagttaaaacgtattcattaggcatgaaacagcgtttgggccttgccgtcgcgctcttgcatgatccggaatttctcattctcgatgaaccgacaaacggccttgatcctcagggaatcattgatttgcgcgaacaccttcagtacttggcgaaaaccttcaacaaaacgattttgatttcgagtcatcttctgtctgaggttgagatgatttgtgatgaatacggcgtcatgaaaaacggagaactcctgcaaattaagagcaatcaccgcgataccgatacggttcgttatcggcttacattaaacggccacgccgatgaagcggctgacctgttgaatgagtaccagtatgcaggcggtctcacggaagataaaaatgagatttatgtcctttgcatggaagaagacattatgaaagtcgttaatctgttaatggagaacaaaataagagttctgcatatgaagcaggaaaaacagtcgatagaacaaagctttctggaattgatcaataaggggtga

SEQ ID NO: 56 - protein, 300 aa, Bacillus licheniformis DSM 13
MSQVEFEGVSKRIKGRPIVQNITFQIAPGTIFGLLGPNGAGKTTLIKMIVGMAKPTSGDIRIDGYSVKSNYEEAAARVGSVVENPSFYEHLTGYQNLKYLGGFHSHVSKERIEEIVQLVDLTGSIHKPVKTYSLGMKQRLGLAVALLHDPEFLILDEPTNGLDPQGIIDLREHLQYLAKTFNKTILISSHLLSEVEMICDEYGVMKNGELLQIKSNHRDTDTVRYRLTLNGHADEAADLLNEYQYAGGLTEDKNEIYVLCMEEDIMKVVNLLMENKIRVLHMKQEKQSIEQSFLELINKG

SEQ ID NO: 57 - DNA, 1200 bp, Bacillus licheniformis DSM 13
  gene (1)..(1200): yciC - unknown function - similar to unknown function proteins - BLi00765
atgagtaaaaagattcctgtgacagtattaagcggatacctgggggcgggtaaaacgacgctgctgaatcacatattgcaaaaccgccaagggctgaaagtcgccgttattgttaatgacatgagcgaagtcaacattgatgcagggcttgtcaagcaaggcggcctgtcgaggacggatgaaaagctggtcgaaatgtcaaacggctgcatttgctgcacactcagagaggatttgctggttgaagtcgaaaagctggcgaagcagggaaatattgattatatcgtgatcgaatcgaccggtatcagcgaaccgatcccggtcgctcagacgttctcttacatcgacgaagaattgggtattgatttgacccggttctgccgcttggacacaatggtgacggttgtcgacgccaatcgtttttggcacgattttcagtcgggggacagcctgctggaccggaaagaggctgtcggcgaagaggatgaacgcgacatcgccgatctgctcatcgatcaaatcgagttctgcgatgtgctgattttgaataaatgcgaccttgtctccgaggaagatctgcaaaagcttgagaaagtgctgaaaactttgcagcctaaagcaaacatcatcagagcggtcaaaggagaggtcgatccgaaggccgtcttaaacaccggtctcttcaattttgacgaagcgagcggttcagcaggctggatcagggaattaaaccaaggacaccaccagcatacgcctgagactgaagaatacggaatatcttcgtttgtatacgaagcgcgccgtccttttcatacagagcggttttataactggattcattcgctgccggaaaacgtcgtcagggcgaaaggaatagcctggtgtgcgacgcgcaacacccttgcccttttgatgtcgcaggcgggtccttccgtgtcgctggaaccgatctcttattgggtggccgcactgtccaaaccggaacaggaacaaattctcagacagaaccctgagcttttaaaagaatggaatgaagaattcggagaccgccatacaaagctcgtctttatcggattggatctgtcgccgtctgaaattaccgcagaagcggatcgatgccttttaacagattcggagatggcggccgactggtcgcttttatctgacccgttcgattggcagattgaaagagctagataa

SEQ ID NO: 58 - protein, 399 aa, Bacillus licheniformis DSM 13
MSKKIPVTVLSGYLGAGKTTLLNHILQNRQGLKVAVIVNDMSEVNIDAGLVKQGGLSRTDEKLVEMSNGCICCTLREDLLVEVEKLAKQGNIDYIVIESTGISEPIPVAQTFSYIDEELGIDLTRFCRLDTMVTVVDANRFWHDFQSGDSLLDRKEAVGEEDERDIADLLIDQIEFCDVLILNKCDLVSEEDLQKLEKVLKTLQPKANIIRAVKGEVDPKAVLNTGLFNFDEASGSAGWIRELNQGHHQHTPETEEYGISSFVYEARRPFHTERFYNWIHSLPENVVRAKGIAWCATRNTLALLMSQAGPSVSLEPISYWVAALSKPEQEQILRQNPELLKEWNEEFGDRHTKLVFIGLDLSPSEITAEADRCLLTDSEMAADWSLLSDPFDWQIERAR

SEQ ID NO: 59 - DNA, 1092 bp, Bacillus licheniformis DSM 13
  gene (1)..(1092): citA - citrate synthase I (minor) - BLi01010
atggtttatcatgggttaaaagggattacttgtgttgaaacagccatcagccgcattgacggtgaaaaaggaaagctgatctacagagggtacaatgctgatgaactggcgctttcgtccacatttgaagaagtcgcttacctgattctcaacggggcatttccgacggaagctgaactcagcagattcaatgagctgctcgcttcatttcgtgtgcttccagaagcatgcgagcgattgatccgcagtcttccggccgaattagacgatatggcggttctgcgcacggccgtctcttcattcggggacatatcgtttgcgttcaagccgacaatggagcaggccctcaggctgattgcggccgtcccgtcaattatcgcgttcagaaagaggctggctgatggagaagagcctgttcagccgctggatgagctaggttttgtggaaaattatttttacatgctgaaaggaaagcgtccgacagaagctcagaaaaaagcgcttgaaacgtacatgattcttgcgatggaacatggcatgaacgcttcaaccttttcggcaagggttacggtgtctacggaatccgatcttgtttctgccgtcacatctgcacttggcacgatgaaaggacctttgcacggcggagcgccgtctgcggtgacggatatgctggaaggcatcggggaaaaacagcacgctgaaagctatataagagaaaagcttgaaaaaggcgagagactgatgggatttggacaccgcgtctataaaacgcatgatccgagagcgaaggctttaagggtgaaagccgaggaaattgccgggggcgacagggatatcgaccttgctcttcatgttgaaaatacggcgatccgccttttggaagaatacaagccgggccgcaagctttatacgaatgtcgaattttacgcagccgctgtgatgaaggcgatcgattttgatgccgcattgttcacccctactttttcggcagcgagaatggtcggctggtgcgcccatgtgcttgagcaggctgacaacaacatgattttcaggccatcggctaaatatgtcggagaattggtctcacagtaa

SEQ ID NO: 60 - protein, 363 aa, Bacillus licheniformis DSM 13
MVYHGLKGITCVETAISRIDGEKGKLIYRGYNADELALSSTFEEVAYLILNGAFPTEAELSRFNELLASFRVLPEACERLIRSLPAELDDMAVLRTAVSSFGDISFAFKPTMEQALRLIAAVPSIIAFRKRLADGEEPVQPLDELGFVENYFYMLKGKRPTEAQKKALETYMILAMEHGMNASTFSARVTVSTESDLVSAVTSALGTMKGPLHGGAPSAVTDMLEGIGEKQHAESYIREKLEKGERLMGFGHRVYKTHDPRAKALRVKAEEIAGGDRDIDLALHVENTAIRLLEEYKPGRKLYTNVEFYAAAVMKAIDFDAALFTPTFSAARMVGWCAHVLEQADNNMIFRPSAKYVGELVSQ

SEQ ID NO: 61 - DNA, 879 bp, Bacillus licheniformis DSM 13
  gene (1)..(879): alsR - transcriptional regulator of the alpha-acetolactate operon - BLi03849
atggagctgcgccatcttcgttattttatggttgttgccgaagaactgcatttcggaaaagcagctcttcggctgaacatgactcagcctccgctcagccagcagattaaacagctagaaagtgaaataggcgtcaccttgctgaagaggtcaaaaagagccgtcaggctaacggcggcaggcggggtctttttaaaacaaatcaaggaaggactttcccagattgatcaggccgtcgacatggctcagcggacggcccgcggggagctgggaagactcgtgatcggctttgtcggctccgcaacatacgagatcatgcctcccatcatcagggaataccgaaaccggttcccgtccgtgcagatcgatcttcgggagctttcaacccccaatcagatcgatgcactgctaaacgggcatattgacatcggggtgctgcatcccccgttgggaaacgacgaactgaagtcatacacggtaaaaaaaagccattgtgtccttgcacttccaaaacaccacccgttgacagaaaaagcccgggtacatttgaaggatcttgaagaagaatcgctgatcgtcattgccaaagaagcgtggccctctttatacacggaattcaatttcttgtgtgaaaaagccggatttatccccaacatcgcccaggaagctacagaataccaaatggtgatcggcctcgtatcggccggaatgggaatcgccgtcgtgcctacggcggctaaacgattatttaaccttgatgttgtatacaaagaaatcgaagactttccccttcgcgccgaatggattaccgcacatcgaaaggacaaccgcaatccggcacttaagcatttcatcgaaatctcagatcaacactcatag

SEQ ID NO: 62 - protein, 292 aa, Bacillus licheniformis DSM 13
MELRHLRYFMVVAEELHFGKAALRLNMTQPPLSQQIKQLESEIGVTLLKRSKRAVRLTAAGGVFLKQIKEGLSQIDQAVDMAQRTARGELGRLVIGFVGSATYEIMPPIIREYRNRFPSVQIDLRELSTPNQIDALLNGHIDIGVLHPPLGNDELKSYTVKKSHCVLALPKHHPLTEKARVHLKDLEEESLIVIAKEAWPSLYTEFNFLCEKAGFIPNIAQEATEYQMVIGLVSAGMGIAVVPTAAKRLFNLDVVYKEIEDFPLRAEWITAHRKDNRNPALKHFIEISDQHS

SEQ ID NO: 63 - DNA, 1380 bp, Bacillus licheniformis DSM 13
  gene (1)..(1380): putative protein - putative ammonium transporter - BLi01175
atgaaaaccaaggtatctgccgtcctgttcctcagttttttcctcgcagcatcagctctggccgccgaaccgacggtgcaatcggtcaatgactcggtcaacatgacatgggtagccatcggcactctgctcgtcttttttatgcacgccggttttgcgatggttgaatcaggctttacaagagctaaaaacacgttaaacattttaatgaaaaacttcctgacgatctctatcggctccatcctttatttcttcgccggatacgcgttcatgttcggggacagcgccggcgggttcatcggcacaagcggttttgctctagcgggtgatcaggatgtcggatttttcgtctttcaggccgtgtttgccgctacgtgcgccaccattatttccggcgctgtcgctgaacggatgaagcttggcagctacatgatcttgacgctgtttatgaccggtctcatctatccggttgtcggccattggacttggggggaaggctggctgtacgacctcggatttgtcgattttgccggatcttcaatcgttcacttgacaggcgctgcgggcgcagttgcggctgtcatgtttctcgggccgcggatcggaaaatatacaaacggcaaggtcaacgccattccagggcacagcattccgctgggcgctctcggcgtctttatcctgtggttcggctggtttgggttcaacggcggaagcacgcttgcggctgatcccgccgccgtcccgcacgtcatcaccacaacccttttagcggcatcgggcggagtcgtcagctcggcgctgttcacttatttccgctacggcaggatcgatccttccctgacgctgaacggcgcgctaggcggccttgtcggaattaccgcgggcgcggacggcgtttcatttacaggctcgatattgatcggattcatcgccggtgtcattttagttgtcggcgttcagtttatcgaccaggtgctgaagcttgacgatcccgtcggcgcccttgccgttcacggtatttgcggtgtttgggggacgcttgcagtcgggcttttcaatacatcgagcggtctcttttacggaggaggagccgcacagcttggcattcaagccgtcggcattttgtcgatcgcggcctggaccctcgctgccacatacgctgtcgccgcggcaatgaaagccctcgctccgatcagggtatcaaaagaggaggaaatttccggactagattatgccgaacacggttcttccgcgtacgagtataaagaaagctacatcgatcaggacgatgttgacgtaagctccgctttcggaacggatcttgtctcacggctcaacaatttaaagcctggaccttcttccaaaactgaacaggtgtga

SEQ ID NO: 64 - protein, 459 aa, Bacillus licheniformis DSM 13
MKTKVSAVLFLSFFLAASALAAEPTVQSVNDSVNMTWVAIGTLLVFFMHAGFAMVESGFTRAKNTLNILMKNFLTISIGSILYFFAGYAFMFGDSAGGFIGTSGFALAGDQDVGFFVFQAVFAATCATIISGAVAERMKLGSYMILTLFMTGLIYPVVGHWTWGEGWLYDLGFVDFAGSSIVHLTGAAGAVAAVMFLGPRIGKYTNGKVNAIPGHSIPLGALGVFILWFGWFGFNGGSTLAADPAAVPHVITTTLLAASGGVVSSALFTYFRYGRIDPSLTLNGALGGLVGITAGADGVSFTGSILIGFIAGVILVVGVQFIDQVLKLDDPVGALAVHGICGVWGTLAVGLFNTSSGLFYGGGAAQLGIQAVGILSIAAWTLAATYAVAAAMKALAPIRVSKEEEISGLDYAEHGSSAYEYKESYIDQDDVDVSSAFGTDLVSRLNNLKPGPSSKTEQV

SEQ ID NO: 65 - DNA, 1032 bp, Bacillus licheniformis DSM 13
  gene (1)..(1032): ycdH - unknown function - similar to ABC transporter (binding protein) - BLi03213
atgaaaaaaacattcggaatagcatctgcattcattcttgctgccggattaacagcgggttgctcaagcaatggagcttccagtgaacaaaaagaaaataaagacacgctggacatctatacaaccatttatccgcttgaagacttcaccaaaaagatcggcggagaatatgtgaacgttaaaagtgtttacccgccaaacgttgaagcgcacacttacgagccaagctcaaaaacgatggcggaaatttccgactcggatgcctttatatactctggagtcggtgttgaaggattcgcagacaaagctgtaagcacacttaaaaacagcgacgtcaaaatcgtcaaagccggggagggcattgaccttctgtcacgtgaagaagaaggcgaacatgatgagcatgcacatggagaagaacacggtgagcacgatgagcatgcacatgaagaagaacatgaacacggagaagccgaaggtcacgatcatcatcacgaccatggagatcatgacccgcacgcatggcttgatcctgtccttgctgaaaaaatggccgaaaacattaaagaccaactcgtaaaattagaacctgaacacgaaaaaacgtttacgaaaaactatgagtcattaaaaaaagacttggagcagcttgacaaagacttcaaacaaacagtcgacaaagctgataaaaaagaaattctcgtctcacatgccgcctacggctactgggaaaaacgctacggcattaagcagataagcgttctcggcctttcgcctactgaggaaccttcacaaaagcagcttgaaaacattgttaaaacagcgaaagaacataatattaaatatatcatttttgaaagcaatgtcagcagcaaaatctccgagatcatcaaaaaagaaatcggagcggaaagtctgacgatgaaaaaccttgaatctgtaacaaaagaagacattgaaagcggcaaagattataccgccatcatgaaagaaaacctggacgtcttgaaaaaagctttatcaagcaaataa

SEQ ID NO: 66 - protein, 343 aa, Bacillus licheniformis DSM 13
MKKTFGIASAFILAAGLTAGCSSNGASSEQKENKDTLDIYTTIYPLEDFTKKIGGEYVNVKSVYPPNVEAHTYEPSSKTMAEISDSDAFIYSGVGVEGFADKAVSTLKNSDVKIVKAGEGIDLLSREEEGEHDEHAHGEEHGEHDEHAHEEEHEHGEAEGHDHHHDHGDHDPHAWLDPVLAEKMAENIKDQLVKLEPEHEKTFTKNYESLKKDLEQLDKDFKQTVDKADKKEILVSHAAYGYWEKRYGIKQISVLGLSPTEEPSQKQLENIVKTAKEHNIKYIIFESNVSSKISEIIKKEIGAESLTMKNLESVTKEDIESGKDYTAIMKENLDVLKKALSSK

SEQ ID NO: 67 - DNA, 1548 bp, Bacillus licheniformis DSM 13
  gene (1)..(1548): trpE - anthranilate synthase - BLi02403
ttgagtctgcaaaccgattttgcctctttttcagaggattcttcacactttaaaacgataccgattattgagagtttttccgtcgatacgctgacgcccgttcaattggtcgagaagctgcaggaagacatcgtctatttgctggaaagccaggatgaaacctctgcatggtcaagatattctttcatcggccttgacccctttttgacgatcaaagaagaacgcggccggtttcacgcctgcgatcaaaagcagaagaagctttattcggcttcacagttaaaagacgttctcgaatggatgtcggagacgtattcgatcaaagcgcctgagctcgacatcccgtttacaggaggagcggtcggctatttaagctacgatttaatgccgctcattgaaccgtccatcaaagggcacagcaaagaaacggaaatggcaaaatgcctcctgtttgtctgccggacgatgatcgcttatgatcacgaactgaaaaagctgcatttcatccactatacaaggcttgaaggcagtgaaaatagggaggaaaagcgccgcattttcaaaaaggggaaagaagagctcaagcagcttacacacaggctgtcagaccggaaaacgccgaaagagctgtttcttccgcaaggacggcacacggcccccagctttgaagggattcaatcaacatatgagaaatcccggtttttggccgatgtcgatcgcttgaaagaatacattaaagcgggagacatttttcaaggcgtcctttcgcaaaggttcgacattccggtgaaggtcggttcttttgagttatacagagtgctgagaatgatcaacccgtccccgtacatgtactacatgaggcttcctgaccgcgatctcgtcggaagctcgcccgaacgtctgatccatgttcaaaacggccatcttgaaatccatccgatcgccggcacaaggaaaaggggcggtgatcaggctgaggataagcggcttgaaaaagaactattggctgatgagaaagaaaaagcggagcattacatgcttgtcgatttggcgagaaatgatatcggcagagtagctgagtacgggtctgtccaagtgcccgagtttacgaaaatcgtttccttttcgcacgtcatgcacatcatttcgatcgtaaccggcactttaaaagcgggtgtccacccggttgacgctttaatgagcgcatttccggccggaactttgaccggcgctccgaaaatccgcgcgattcagcttttaaatgagcttgagcccgaaccgagggaaacatacggaggatgcattgcttacatcggatttgacggaaatatcgattcgtgcatcacgatccggacgatgagcgtgaaaaacggagtggcgtccattcaggccggggcgggcatcgtagccgactccgtgcctgaaaaagaatgggaagagacctgcaataaagcaggggctctgttaaaaacgattcaaatcgcagaagaaacgtttctcgaaaagggggaggacggcaatgaacgaactgctgaaatcatgcgttaa

SEQ ID NO: 68 - protein, 515 aa, Bacillus licheniformis DSM 13
  misc_feature (1)..(1): Xaa can be Leu or Met.
XSLQTDFASFSEDSSHFKTIPIIESFSVDTLTPVQLVEKLQEDIVYLLESQDETSAWSRYSFIGLDPFLTIKEERGRFHACDQKQKKLYSASQLKDVLEWMSETYSIKAPELDIPFTGGAVGYLSYDLMPLIEPSIKGHSKETEMAKCLLFVCRTMIAYDHELKKLHFIHYTRLEGSENREEKRRIFKKGKEELKQLTHRLSDRKTPKELFLPQGRHTAPSFEGIQSTYEKSRFLADVDRLKEYIKAGDIFQGVLSQRFDIPVKVGSFELYRVLRMINPSPYMYYMRLPDRDLVGSSPERLIHVQNGHLEIHPIAGTRKRGGDQAEDKRLEKELLADEKEKAEHYMLVDLARNDIGRVAEYGSVQVPEFTKIVSFSHVMHIISIVTGTLKAGVHPVDALMSAFPAGTLTGAPKIRAIQLLNELEPEPRETYGGCIAYIGFDGNIDSCITIRTMSVKNGVASIQAGAGIVADSVPEKEWEETCNKAGALLKTIQIAEETFLEKGEDGNERTAEIMR

SEQ ID NO: 69 - DNA, 1017 bp, Bacillus licheniformis DSM 13
  gene (1)..(1017): trpD - anthranilate phosphoribosyltransferase - BLi02402
atgaacgaactgctgaaatcatgcgttaacggacggacactgaatgaaatcgaggcacacagcatcatgcgcgacatgatgaccggctcgctgagcgctgcggaaatcggaggcctcctgtccgttctcgcctacaggggagaaacggctgaagagattacgggatttgtcagagccatgcgggagcaggcgcatacaatagacggaccggaacatgttgttgatacgtgcggaaccggaggagacggaagctccaccttcaacatttccacagcggctgcgatcatcgcttcatcagccggggcaaaaatcgcaaaacacggtaatcgatccgtttcttcaaaaagcgggagcgctgacgttctcgaatatctcggcgtttctattcagacgtcgcctgaagaaacgattaagagcattgaacgccgaaatatgggattcctgtatgcgccaagctatcactcttccatgaagcacgtggcaggcgcgaggaaggatctggcttttagaaccgtattcaatctgctcggcccgctcagcaacccgatgaaggccaaacgccaggtgatcggcgtctactcgatcgaaaaagcaaagcttatggcccgcgctcttgaagcgtttgaaccggagcacgtattgtttgtttcgagccgggacggtcttgatgaactgtcgatcacatcgccgacagatgtaattgaactgaaaggcggcaaaacatacgaatacaccgtcgagccgggcgatgttggactgcctctcggaaggctgagagacattcaggtggcgacgcctgaagaaagcggccgtctgatcgtcgatattttgaaaaaccgcggtccggactcggctgttcatatcgcagcgtttaatgccggagccgctttatacgtcgcagaacaggcagcggatttaaaagagggcgttgcgctcgctttagaagctgttgctggcggtgaagcgctcaatcagctggagcgattaaaacagaaagaggaagagttgtatgcttaa

SEQ ID NO: 70 - protein, 338 aa, Bacillus licheniformis DSM 13
MNELLKSCVNGRTLNEIEAHSIMRDMMTGSLSAAEIGGLLSVLAYRGETAEEITGFVRAMREQAHTIDGPEHVVDTCGTGGDGSSTFNISTAAAIIASSAGAKIAKHGNRSVSSKSGSADVLEYLGVSIQTSPEETIKSIERRNMGFLYAPSYHSSMKHVAGARKDLAFRTVFNLLGPLSNPMKAKRQVIGVYSIEKAKLMARALEAFEPEHVLFVSSRDGLDELSITSPTDVIELKGGKTYEYTVEPGDVGLPLGRLRDIQVATPEESGRLIVDILKNRGPDSAVHIAAFNAGAALYVAEQAADLKEGVALALEAVAGGEALNQLERLKQKEEELYA

SEQ ID NO: 71 - DNA, 759 bp, Bacillus licheniformis DSM 13
  gene (1)..(759): trpC - indol-3-glycerol phosphate synthase - BLi02401
atgcttaatcaaattattgatagaaaacgagaagacatcctaaagatcaagctgtcggaagatttaaaactgccaaagcggtcatttaaaaaagcgctcctttctccgaaccggtttgtcgctttaattgcggaggtgaaaaaagcttctccgtcaaaaggcgtgattcaggagggttttgagcccgtcaagatcgcaaaacaatacgaacaggctaaagccgattgtttgtctgttctgacagatacacctttttttcaagggaagaacagttatttgtcagatgtgaaacgctcggtttccctgcctgtgctgcgcaaggattttatcattgattccattcaggttgaagaagcggaccgcatcggagcggatgcgatcctgctgatcggtgaagcgcttgaaccgcaaaagctgtttgaactctatcagcaagccgttgaaaaaggcatggatgtgctcgtcgaagtacatggcgaagagacgcttgagggcattttaaatgtctttacacccgaaatcatcggcgtaaacaatcgaaatctaaaaaactttgaaacgaccgtcggacaaacggagcgaatggcgaaactcgttccgccggggaccgttctcatcagcgaaagcggcatcggaaaatcggaagacctgacttttgtaaaaacgtgcggagcccaagccgtgcttgtcggcgaatcgctgatgagagagacgtcacagctgaaggctgtctatgcgttattcggagaggacggctga

SEQ ID NO: 72 - protein, 252 aa, Bacillus licheniformis DSM 13
MLNQIIDRKREDILKIKLSEDLKLPKRSFKKALLSPNRFVALIAEVKKASPSKGVIQEGFEPVKIAKQYEQAKADCLSVLTDTPFFQGKNSYLSDVKRSVSLPVLRKDFIIDSIQVEEADRIGADAILLIGEALEPQKLFELYQQAVEKGMDVLVEVHGEETLEGILNVFTPEIIGVNNRNLKNFETTVGQTERMAKLVPPGTVLISESGIGKSEDLTFVKTCGAQAVLVGESLMRETSQLKAVYALFGEDG

SEQ ID NO: 73 - DNA, 657 bp, Bacillus licheniformis DSM 13
  gene (1)..(657): trpF - phosphoribosyl anthranilate isomerase - BLi02400
atgaagaaaccgagtctaaaatattgcgggattcattcattggaagatttaaaggtgacagcccgctcgaaagccgattatctcggatttattttcgccgaaagcaaacggtcggtagacccggcggatgtgaaacggtggtgcggcgaaacggatacgccaggtaaaaagcttgtcggcgtttttgttaatgaaaacatgagccgaatggctgaaatcgtaaaagatgcaggcttggacgtgatccagcttcacggagatgaaacggcggcagacatcaagcggcttaaaagcatgacagactgcgagatatggaaggcgctccctcacgggaatgataccgtacagagcatggcttctttcgcaccttacatcgacgggtatgtcatcgactcgtctgtcaaagggatgagagggggaacgggagtttcgttttcgtgggacagcgtcccgctgtatatagatgcagcccaaagagaaggcaagcgcctgtttatcgccggaggcgtgaacccggacacaatcgcagatcttttaaagcggcgtccgccgggaatcgacttggcaagcggaatcgaagaaagaggcagaaaaagcgaaaagctgatcagccttttagaagaaaggatgttcgaacatgtattcatatccgaatga

SEQ ID NO: 74 - protein, 218 aa, Bacillus licheniformis DSM 13
MKKPSLKYCGIHSLEDLKVTARSKADYLGFIFAESKRSVDPADVKRWCGETDTPGKKLVGVFVNENMSRMAEIVKDAGLDVIQLHGDETAADIKRLKSMTDCEIWKALPHGNDTVQSMASFAPYIDGYVIDSSVKGMRGGTGVSFSWDSVPLYIDAAQREGKRLFIAGGVNPDTIADLLKRRPPGIDLASGIEERGRKSEKLISLLEERMFEHVFISE

SEQ ID NO: 75 - DNA, 1227 bp, Bacillus licheniformis DSM 13
  gene (1)..(1227): trpB - tryptophan synthase (beta subunit) - BLi02399
atgtattcatatccgaatgaacaaggcaggtttggtgattttggcgggaaattcgtcccggaaacactcatgcagcctttggaagaaatcgaaaaagcgtttcttgaactgaaagacgacccttcatttcaaaaggattatcaaaagcttttaaatgactactcaggcaggccgacggctttgacgttcgcagaccagctcactaagcagctgggcggagcgaagatttacttaaaaagagaagatttaaaccatacaggcgcccacaaaatcaacaatgccctcggccaggcgctcctggccaagaaaatgggaaaaacaaagctcattgctgaaaccggtgcaggccagcacggtgtggcggcggcgactgtcgccgcgaagttcggcttatcatgcatcgtcttcatgggggaggaagatgttgccagacaatcgctgaacgtgttccggatgaagctcttgggagcggaggtcgtcccggtgtcaagcggaaacgggacattaaaagatgctacaaacgaagcgatccgctactgggttcagcattgccaggaccatttttacatgatcggttccgtcgtcgggccgcatccgtatccttatatcgtccgcgaatttcaacggatgatcggcgaagaggcgaaagcgcagttttatcaattggagcaaagcctccccgataaagttgtcgcctgtgtcggaggcggaagcaatgcgatcgggatgttcagcgcgttcattgaagaaactgttgaactgatcggagtcgaagctgcgggaaaaggtgttgacacacctcttcatgcggccacgatcacaaagggtacaaaaggcgtcatccacggttctttgacctacttgattcaagacgagtacggacagatcatcgagccttattccatctcagccggacttgattatccgggagtcggccccgagcacgcccatcttcatcaaacagggcgcgtcacgtatgaatctgtcacagatgatgaagcggtctcggcattaaggctgctgacagagactgagggcattttgccggctattgaatccgcccatgcattggcaaaagcatttgaaatcgctaaagaactgccgagagagaaaagcgttctcgtctgcctttcaggcaggggagacaaagacgtccataccttgatgaaggtgttggaagaagaggtgaatcaagatgttcaatctgaaaaaacagccgtccgataa

SEQ ID NO: 76 - protein, 408 aa, Bacillus licheniformis DSM 13
MYSYPNEQGRFGDFGGKFVPETLMQPLEEIEKAFLELKDDPSFQKDYQKLLNDYSGRPTALTFADQLTKQLGGAKIYLKREDLNHTGAHKINNALGQALLAKKMGKTKLIAETGAGQHGVAAATVAAKFGLSCIVFMGEEDVARQSLNVFRMKLLGAEVVPVSSGNGTLKDATNEAIRYWVQHCQDHFYMIGSVVGPHPYPYIVREFQRMIGEEAKAQFYQLEQSLPDKVVACVGGGSNAIGMFSAFIEETVELIGVEAAGKGVDTPLHAATITKGTKGVIHGSLTYLIQDEYGQIIEPYSISAGLDYPGVGPEHAHLHQTGRVTYESVTDDEAVSALRLLTETEGILPAIESAHALAKAFEIAKELPREKSVLVCLSGRGDKDVHTLMKVLEEEVNQDVQSEKTAVR

SEQ ID NO: 77 - DNA, 210 bp, Bacillus licheniformis DSM 13
  gene (1)..(210): yppF - unknown function - BLi02362
atgcaccatatgaacgtatcatacttaagaaattgttttgccgagatgaaacagtatgagacagactgcatgaacaaactgatggatttcgctaagtttttgtatatccagggacatctgacattaaacgaatttcgcacaagtatgaaagttcttgaagcgaatggcgcacagcaccctgcttatgatatgaatacggacgccagctaa

SEQ ID NO: 78 - protein, 69 aa, Bacillus licheniformis DSM 13
MHHMNVSYLRNCFAEMKQYETDCMNKLMDFAKFLYIQGHLTLNEFRTSMKVLEANGAQHPAYDMNTDAS

SEQ ID NO: 79 - DNA, 711 bp, Bacillus licheniformis DSM 13
  gene (1)..(711): ydfS - unknown function - similar to unknown function proteins - BLi01489
atgagtgaacatattgaagtaatccttcgatcgattcttgcctttggtattttattggtaggatcaagattgttgggaaaacaaacaatttcacaaatgaatatctttgattttattgcatcaattacattaggcgctattactgcaaatctggcttttaatacctctcttaaaattcatcacatggtactggctttttttatatttgtttttgtgatcttagccactgcgtatatatccctttggagtcaaaaaggccgcaaattttttgccggtgatccaactgttgtgattcaaaatggaaaagttcttgaacacaacatgcgaaaaatgcgttatacaattgattatttgaatcaacaacttagagaaaagaatgtattcaaaatcgaagaagtactctatgccattgttgaaacaaatgggaccttaacagtattaaaaaaaccacaattccgaaatgtgacaaaaaaggatttgtgggttgccactacgccagaaggaaaactcccgattgaattaatcatggatgggaaaatgaagaatgaaaacttgaaagataatcaactcactcaggcatggttacaatctgagatgaaaaagcgtggtctatccacagatgaagttttttatgcagtattatcacctgatggaaatatctatatagatacatatgaagatcatatacattcttcttttgataaagaataa

SEQ ID NO: 80 - protein, 236 aa, Bacillus licheniformis DSM 13
MSEHIEVILRSILAFGILLVGSRLLGKQTISQMNIFDFIASITLGAITANLAFNTSLKIHHMVLAFFIFVFVILATAYISLWSQKGRKFFAGDPTVVIQNGKVLEHNMRKMRYTIDYLNQQLREKNVFKIEEVLYAIVETNGTLTVLKKPQFRNVTKKDLWVATTPEGKLPIELIMDGKMKNENLKDNQLTQAWLQSEMKKRGLSTDEVFYAVLSPDGNIYIDTYEDHIHSSFDKE

SEQ ID NO: 81 - DNA, 852 bp, Bacillus licheniformis DSM 13
  gene (1)..(852): hypothetical protein - unknown function - BLi01488
atgcctcccgacagaaaactaattaatgacattcacaaggcaattaacggagagcatagcgccattctctgctataagcaaatagccgaaaatgcccccacagaagatataagaaaacaaatacttgaaattagacaagatgagcaacgacattttgaagagtttagcagaattttcactaacctgacaggcaaaaagccaatccccataatcatagaagaatgccctaaaaagtatagaacaagtctagagtctgcttttaaagatgagcaggagacagtcgatttttatttagatttagcagaccgaatacaaaatcagtcaattaaaaaggtttttcagcgtgcagcagctgatgaacaaaatcatgctgtatggttcttattttttttaaatacatcgtctacacaacaaacacgtcaagttaattatggggcaaaaggagcgttaaattccggtaggctcaccttacctcaaatgcttacctttgccattcaggatgagtacttagctcaagcaagatacaatgaaattctaagaaacttcggggatattcgaacctttgtccaaattaaagaagccgaattacggcatatcaatgcattactttctctttttgaacgataccaagtacagatacctaaagacacctctcaattatatgtcactacaccagtaacccttaaggatgcatatgcttctggggtacaaggagaaattgataatatcgccatgtataacaagtttctaacttttgatcttccagcagatgcaagaacagtttttactcaattacgcaacgcatcactaaatcatttagaggctttccgaagaggtcttgagagaaattaa

SEQ ID NO: 82 - protein, 283 aa, Bacillus licheniformis DSM 13
MPPDRKLINDIHKAINGEHSAILCYKQIAENAPTEDIRKQILEIRQDEQRHFEEFSRIFTNLTGKKPIPIIIEECPKKYRTSLESAFKDEQETVDFYLDLADRIQNQSIKKVFQRAAADEQNHAVWFLFFLNTSSTQQTRQVNYGAKGALNSGRLTLPQMLTFAIQDEYLAQARYNEILRNFGDIRTFVQIKEAELRHINALLSLFERYQVQIPKDTSQLYVTTPVTLKDAYASGVQGEIDNIAMYNKFLTFDLPADARTVFTQLRNASLNHLEAFRRGLERN

SEQ ID NO: 83 - DNA, 594 bp, Bacillus licheniformis DSM 13
  gene (1)..(594): yrkC - unknown function - similar to unknown function proteins - BLi01487
atgtattatgttcctcatatgtatccatatcaatattcttatccttattatgttaatgtgccaatgtacaactgtagaagacagtcagttcaccggactgttcctagtgaagtagagcatgcaaacaaattaagttctgttcgctcatccaaaggtagtaacagcattttgttaaaggattatggaccagaagcgtttgtggttaatatcaatgaagctacgaagcaaaataatacgtaccgtaccgctttatggacaggaactcatttgcaagtcaccttaatgagtctcaatgttggagaagatattggtttggaaatgcaccctaacgttgatcaattcttacgtgttgaacaaggtcagggaatcgttcaaatgggcaagaataaagaccatttaaatttcaaaagaaatgtctatgatgattctgcaatcataatacccgctggaacatggcacaatctaaccaatacaggtagaatcccgctaaaactatactccatatatgctcctcctaaccatccatttggtactgttcatgttactaaaggagatgcattagcttcagaagaagaatacggtcatggcaattga

SEQ ID NO: 84 - protein, 197 aa, Bacillus licheniformis DSM 13
MYYVPHMYPYQYSYPYYVNVPMYNCRRQSVHRTVPSEVEHANKLSSVRSSKGSNSILLKDYGPEAFVVNINEATKQNNTYRTALWTGTHLQVTLMSLNVGEDIGLEMHPNVDQFLRVEQGQGIVQMGKNKDHLNFKRNVYDDSAIIIPAGTWHNLTNTGRIPLKLYSIYAPPNHPFGTVHVTKGDALASEEEYGHGN

SEQ ID NO: 85 - DNA, 954 bp, Bacillus licheniformis DSM 13
  gene (1)..(954): putative protein - putative glycosyl hydrolase/lysozyme - BLi01486
atgggaatcaaaggaatcgacgtatcacactggcaaggtaatatcaattggaagaaagttgcgggggacggtattaaattcgcttttatcaaagcaacagaagggacaacattacaggacaataaatttgtaacgaatatttcaggtgctaacgctgtggggattaaaacgggagcctaccactttgcaagattcggttccaagtcagaagcattggcagaggccaggttctttttgtcagttgcaaataaggtccatctcacgtatccgcttgtgcttgatcttgaagttaatcagcggaatgtcagtaaatcagttttgacagatgcagcagtggcctttttacgggaagttgaaaaagctggttacttcgccatgatatacagcggtaagtctttccttgagaattgccttgacgaatccaagctgaagccatttgcattatgggttgctcgttataacaacacacttggccgtcatgcagatatctggcagtattctgattgtggaagggtcgccggtatttctgggaatgttgatatgaatatttgttatcgtgacgggttacgggctcaggtagccgtgacaactgaaaaagctgctactgtaaaacctgtttcaaacaaaaagccagttaaaacggagacagtttacacagtcaaaaaaggggacacgctttcggaaatcgcacagaaaaacaacacgaccgttaaagctctccaaaatttgaataacattaaagatgcgaataagatttacgttggccagaaattaaagattagcggcagtccttcaacagcatcgaataaaaagcaatattacaccattaaatccggtgacactttgtctgggatctcgaaaaggtttaatacatccattaagacgctgcaagcttggaatggtatcaagaatgccaacaagatttacgctggtcagaaaattcgtgttagataa

SEQ ID NO: 86 - protein, 317 aa, Bacillus licheniformis DSM 13
MGIKGIDVSHWQGNINWKKVAGDGIKFAFIKATEGTTLQDNKFVTNISGANAVGIKTGAYHFARFGSKSEALAEARFFLSVANKVHLTYPLVLDLEVNQRNVSKSVLTDAAVAFLREVEKAGYFAMIYSGKSFLENCLDESKLKPFALWVARYNNTLGRHADIWQYSDCGRVAGISGNVDMNICYRDGLRAQVAVTTEKAATVKPVSNKKPVKTETVYTVKKGDTLSEIAQKNNTTVKALQNLNNIKDANKIYVGQKLKISGSPSTASNKKQYYTIKSGDTLSGISKRFNTSIKTLQAWNGIKNANKIYAGQKIRVR

SEQ ID NO: 87 - DNA, 474 bp, Bacillus licheniformis DSM 13
  gene (1)..(474): yncE - unknown function - BLi03150
atgagtcataagcgaggcgttttatttgaaatcccgaacgggtacgggcgctttcttctcgatatactgaagccggttgaaccggccgatttcaattggctgcttgccgatggcgaatgctatctcgtggaaaacggtgaattgtcagatgaaaatctttttcctcaagatccgcaggtgatgacaggagaagcatttaaaaaaaggctggaagacgcaaggtactatctcatcttcgccgatctgaaggcttttccgaaggatggccatcccgaagatgtcagcacatatgaatcatatatgaaaagcagctgcgagctgattgtgcttacagccgattcgagttttgtcacgatatactgcaaagatcaaaaaaacctggagcttctatatcaaaatgcaaaggactgcggtttcaagcggattgaatatataacggatcacaacgatgccagaacaggaatgcatgtgtag

SEQ ID NO: 88 - protein, 157 aa, Bacillus licheniformis DSM 13
MSHKRGVLFEIPNGYGRFLLDILKPVEPADFNWLLADGECYLVENGELSDENLFPQDPQVMTGEAFKKRLEDARYYLIFADLKAFPKDGHPEDVSTYESYMKSSCELIVLTADSSFVTIYCKDQKNLELLYQNAKDCGFKRIEYITDHNDARTGMHV

SEQ ID NO: 89 - DNA, 1095 bp, Bacillus licheniformis DSM 13
  gene (1)..(1095): yvlB - unknown function - similar to unknown function proteins - BLi03752
atgaagcatgagaaagaacgaattttaaaattggtcgaggaaggaaagctgtctgcaaaagaggcgctgatcctgatcgaaaaacttgaagaagactacaaggaaaccgagagcaaggtgacagccctgtctgaggatgtcattgattcggaggacttctacagcgaaaagaaaaaagaaccgaagccttcgatcggttcaaaactgttcgaatggattgataccgctgtaaaaaaagtaaaagaagcagacttggatttgaatttcgggcactcgtacgatgtccagcatatattccagtttaaagacactgatttttcgactctggatattcagcttgccaacggcagcgtcaaccttgtaccatggagcgattccgacatcagagtcgaatgccaggcaaaagtgtaccgggctgaaaatcaggatgaagcaaggcaggcttttctccagcatatcgactgcggtgtggaaggcgataagttcatgatccgtaccgagaaaaaaacgatgaagaccaaccttaccgtctatataccggacaaagagtacggcaaaatccgctttaaactgtttaacggcccggtcagaggggaacatttaaacgtaaaagagctgtccgccaaaacgacgaacggcgtgctgtcgttctctgctctcacggcaggaaaagcttcattggaaactgcaaatggacaaatcaaactagccgaccatacttgcggagagattgaagcggaaaccatcaatggattgatcgaccttaaaggttcatgcgaggcgcttgacctgcagagctttaacggcaatatcgcggcaacagtcaaaaatccgcactgccggacggcatatgtgaaaacaacgactggaagcgtagagttgaatgttccccgggactgcgctgttacagcggagctgaaaagcaatctcggttctctgtctcatgatctttcagatgcagaaattttaaaggaaaaaaacgagacaatccagaaggaaatgtacctaaaagcaaatcaaaatcatgatcatagcatgaccgtgtttttggaatcgaagacaggatcgatccatttaaaccatacacaggagtga

SEQ ID NO: 90 - protein, 364 aa, Bacillus licheniformis DSM 13
MKHEKERILKLVEEGKLSAKEALILIEKLEEDYKETESKVTALSEDVIDSEDFYSEKKKEPKPSIGSKLFEWIDTAVKKVKEADLDLNFGHSYDVQHIFQFKDTDFSTLDIQLANGSVNLVPWSDSDIRVECQAKVYRAENQDEARQAFLQHIDCGVEGDKFMIRTEKKTMKTNLTVYIPDKEYGKIRFKLFNGPVRGEHLNVKELSAKTTNGVLSFSALTAGKASLETANGQIKLADHTCGEIEAETINGLIDLKGSCEALDLQSFNGNIAATVKNPHCRTAYVKTTTGSVELNVPRDCAVTAELKSNLGSLSHDLSDAEILKEKNETIQKEMYLKANQNHDHSMTVFLESKTGSIHLNHTQE

SEQ ID NO: 91 - DNA, 1182 bp, Bacillus licheniformis DSM 13
  gene (1)..(1182): putative protein - putative ABC transporter (amino acid permease) - BLi03212
atgaaggaaaacctgattccggtcactgttttaagcggttatctagggtccgggaaaacgacgctgctgcagcatgtcctgcatgaagcgcacggcttgaaaatcgccgtgatcgtcaatgatatgagcgagattaatatcgacggctccctcgtgaaaaaaggtgacctcatccgcacggatgaaaagcttgtcgaaatgcagaacggctgcatttgctgtacgctgagagacgatctgctcatcgaagtcaaaaagctggcggagaacggcggaatcgattatattctcattgaatcttccggtatcagcgagccgattcccgtcgcccaaacctttacgtacaaagatgaggagctgaatattgatttaggcaaatattgcaggcttgatacaatggtcaccgtcgtcgacggcagccgtttttttgaggacttcgcttcgggcgagagcctattggaacggaagcaggcggcagatgaaacggacagccgggaagtggccgaccttttaattgagcaaattgaatttgccgatgtcctcattttgaataaaactgatttactcaatgaagaagagcaggaaaagctgttctggtccctgagaaaactgaatccggactgcagaatcatttctgcggcattcgcaaaagtgccgctttcggccatcatgaatacgaaatcgttcgattttgaaaaagcaagtcaatcagcgggatggatcaaagagctgaatgaagagcacacacctgaaacagaggaattcggaatctcttctttcgtctatcaaaagcaaaggcctttccatcctgaaagactgaagcgctggcttgaaaactggccgccgcaaattatcagagcaaaaggcttcttctggcttgcttctcgaaatgacacagcaggccttctgtcccaggcaggctcttccctgatgatccaaagcgcgggcccctggattgcagcgtctaccgaggaagaacaaaagctcgcgtttgaagaggaccctaatttgaaagcggactgggatgaaacatacggagaccggaaaacggaaatcgtttttatcggcattgacatgaatcaggaagacattatccgctccctggatcagacccttcttacagataaagaaatgaattcagactggtcggccttacttgattcactgcctgagtatatttaa

SEQ ID NO: 92 - protein, 393 aa, Bacillus licheniformis DSM 13
MKENLIPVTVLSGYLGSGKTTLLQHVLHEAHGLKIAVIVNDMSEINIDGSLVKKGDLIRTDEKLVEMQNGCICCTLRDDLLIEVKKLAENGGIDYILIESSGISEPIPVAQTFTYKDEELNIDLGKYCRLDTMVTVVDGSRFFEDFASGESLLERKQAADETDSREVADLLIEQIEFADVLILNKTDLLNEEEQEKLFWSLRKLNPDCRIISAAFAKVPLSAIMNTKSFDFEKASQSAGWIKELNEEHTPETEEFGISSFVYQKQRPFHPERLKRWLENWPPQIIRAKGFFWLASRNDTAGLLSQAGSSLMIQSAGPWIAASTEEEQKLAFEEDPNLKADWDETYGDRKTEIVFIGIDMNQEDIIRSLDQTLLTDKEMNSDWSALLDSLPEYI

SEQ ID NO: 93 - DNA, 390 bp, Bacillus licheniformis DSM 13
  gene (1)..(390): putative protein - putative nitrogen regulatory protein P-II - BLi01176
gtgaaaaaaatcgaaacgattatacgccccgaacagttcccccgcctccgcaaaaagcttgaagaggttggcataaacggattaaccgtctcagaggttgccggctgcggccagcagcgggggaagcaggctctgtttcgcggcacctcctacgaaatcaagctgctgcctaaagtgaaagtcgaaatggtgattgaatcggagagcgttgatgaaattatcaaatcgaaagcacatgttcgacaaatacggtcggcgacggaaaaatctttgtccttccaattgaaaacgcgatccgaatccggacaggggaatcaggaaaggaagcgattgtctaaaacgaccgattccctcctgctctcttttagtcgatactttaagaaagggtga

SEQ ID NO: 94 - protein, 129 aa, Bacillus licheniformis DSM 13
  misc_feature (1)..(1): Xaa can be Val or Met.
XKKIETIIRPEQFPRLRKKLEEVGINGLTVSEVAGCGQQRGKQALFRGTSYEIKLLPKVKVEMVIESESVDEIIKSKAHVRQIRSATEKSLSFQLKTRSESGQGNQERKRLSKTTDSLLLSFSRYFKKG

SEQ ID NO: 95 - DNA, 366 bp, Bacillus licheniformis DSM 13
  gene (1)..(366): hypothetical protein - unknown function - BLi00719
gtgaatcaaatacagctggaagaaaagttgtctattttactaacgagtcgagtgattacaaaaaatgctttcaacacaacattgaacacatttgttttcttatcagaaaagctgaaaaaggaggtgatcaaagactctgaaatgttttggacgcacatggcgatggctttaactagaattaaggagggagaaagcgaagaagttcctgcaaatgccatcatcaatgaaatgtatgaaacaccatataaaagtgcaatagaagaagtgatagactttatacattcactggcaggtctagaacttccagaaggtgagctcgtttatttttatcttcatcttcataaagtcatagaatctaataaatag

SEQ ID NO: 96 - protein, 121 aa, Bacillus licheniformis DSM 13
  misc_feature (1)..(1): Xaa can be Val or Met.
XNQIQLEEKLSILLTSRVITKNAFNTTLNTFVFLSEKLKKEVIKDSEMFWTHMAMALTRIKEGESEEVPANAIINEMYETPYKSAIEEVIDFIHSLAGLELPEGELVYFYLHLHKVIESNK

SEQ ID NO: 97 - DNA, 327 bp, Bacillus licheniformis DSM 13
  gene (1)..(327): yvlA - unknown function - BLi03753
atgacgagaaacaacgcgctgatttcttcaatttgttattttagtgttttttttgccccgcttattttgccaatcgcagcgtattttataacagatgatcaagaaacgaaaaagcatgcgatgcgttcgcttatttcgcatattattccgtttataagcgtagtcatattggcagcaggcgcgataagcggtacgcttatctttggtgacggcggttatttggcggtcattttcatgatcggaggtttcgtttttttcgggatcatcagtttgattattgtggtctggaacgtagttcaagggattaaaatgctgactcaggcctaa

SEQ ID NO: 98 - protein, 108 aa, Bacillus licheniformis DSM 13
MTRNNALISSICYFSVFFAPLILPIAAYFITDDQETKKHAMRSLISHIIPFISVVILAAGAISGTLIFGDGGYLAVIFMIGGFVFFGIISLIIVVWNVVQGIKMLTQA

SEQ ID NO: 99 - DNA, 333 bp, Bacillus licheniformis DSM 13
  gene (1)..(333): tnrA - transcriptional pleiotropic regulator involved in global nitrogen regulation - BLi 01490
atgacagtcgaagatctttcttataaagataaaaaagtgatttcaattggcattgtcagcgagctgacgggtctgtcggtcaggcagatccgctattacgaagaacgcaagctgatctatccgcagcgctctacaagaggcacgcgcaaatattcctttgctgacgtcgaacggctgatggaaatcgcaaacaagcgggaagacggtgttcagaccgcagaaatcctcaaagatatgcgcaaaaaagaacaaaggctaaaaaacgaccagcagctcagaaagaaaatgctcgaaggccagcttaacgctcattttaaatacaaaaaccgttaa

SEQ ID NO: 100 - protein, 110 aa, Bacillus licheniformis DSM 13
MTVEDLSYKDKKVISIGIVSELTGLSVRQIRYYEERKLIYPQRSTRGTRKYSFADVERLMEIANKREDGVQTAEILKDMRKKEQRLKNDQQLRKKMLEGQLNAHFKYKNR

(19) United States
(12) Patent Application Publication
(10) Pub. No.: US 2010/0112552 A1
(43) Pub. Date: May 6, 2010

(54) NUCLEIC ACID-BINDING CHIPS FOR DETECTING NITROGEN DEFICIENCIES AS PART OF BIOPROCESS CONTROL

(75) Inventors: Stefan Evers, Mettmann (DE); Jorg Feesche, Erkrath (DE); Karl-Heinz Maurer, Erkrath (DE); Thomas Schweder, Greifswald (DE); Michael Hecker, Greifswald (DE); Birgit Voigt, Greifswald (DE); Britta Jurgen, Greifswald (DE); Le Thi Hoi, Hanoi (VN)

Correspondence Address: Ratner Prestia, P.O. Box 980, Valley Forge, PA, 19482, US

(73) Assignee: Henkel Kommanditgesellschaft Auf Aktien, Dusseldorf (DE)

(21) Appl. No.: 11/991,761

(22) PCT Filed: Sep. 7, 2006
(86) PCT No.: PCT/EP2006/008726

§ 371 Date: Jan. 18, 2010

(30) Foreign Application Priority Data
Sep. 8, 2005 (DE) 10 2005 042 572.0

(51) Int. Cl. C12Q 1/68; G01N 33/00

(52) U.S. Cl. 435/6; 422/68.1

(57) ABSTRACT

The invention relates to nucleic acid-binding chips for monitoring bioprocesses, specifically for detecting nitrogen deficiencies. Said chips carry probes that are sensitive to at least three of the following 50 genes: kdgR, citA, htrA, ycn1, yppF, trpB, ggt, alsR, glnA, nrgA, yciC, yvtA, nrgB, ycnJ, glnR, yvlA, yncE, yvlB, trpF, ydfS, trpD, ycnK, trpB, trpC, nasD, ycdH, nasC, nasB, trpE, pckA, nasF, yrkC, and tnrA or the homolgs to SEQ ID NO: 91, 41, 53, 19, 55, 47, 21, 17, 9, 85, 45, 49, 95, 63, 15, 93, or 81 at a maximum of 80 different probes that are specific of nitrogen metabolism. The invention also relates to the use of corresponding gene probes, especially on the aforementioned chips, to corresponding methods and possible uses.

The present invention relates to nucleic acid-binding chips for monitoring bioprocesses, specifically for detecting nitrogen deficiencies, as well as the use of corresponding gene probes, especially on the aforementioned chips, to corresponding methods and possible uses, which depend on such probes and chips.

In the industrial utilization of biological processes, one is faced with the very fundamental problem of monitoring their progress, of achieving the desired result, of conserving resources and/or of achieving an optimal result in a given time. Biological processes are understood to mean, for example, the culture of microorganisms on an agar plate or in a shaker culture, particularly however, their fermentation or the production of raw materials by the fermentation of microorganisms. There is extensive prior art for this in regard to both single cell eukaryotes, such as yeasts or streptomycetes, as well as to Gram-negative or Gram-positive bacteria.

Processes of this kind are monitored firstly by observing the properties and requirements of the concerned organisms, which change in the course of the process, these changes being reflected, for example, in the optical density and viscosity of the medium, in absorbed or released gases, in pH changes or in changing nutrient requirements. The measurement of enzymic activities via suitable assays, for example the detection of activities of interest in the culture supernatant, may also be included here.

Secondly, various techniques have been developed in recent years in order to monitor the metabolic processes of the organisms in question at the level of gene expression. A common method for this is the use of genes for readily detectable proteins as indicators of the activity of the promoters of the actual genes of interest (promoter analysis, gene expression analysis). For this, appropriate apparatuses (“(bio)sensors”) have also been developed.

Other techniques are concerned with the detection of the proteins of interest, or of the mRNA coding for these proteins. These techniques include (1.) proteome analysis, i.e. observing the change in provision of the cells in question with proteins, which analysis is usually carried out by way of two-dimensional gel electrophoresis of cell lysates, (2.) analysis of the mRNA formed (transcriptome) by way of a “genomic DNA array” generated in an analogous manner, and (3.) chip technology.

The last technique is in a comparatively early stage of development. Whereas the two methods mentioned first are ultimately based on quantitative isolation procedures and time-consuming analyses of the macromolecules in question, chip technology is based on the principle of attaching probes for proteins or for nucleic acids on physically readable carriers (chips), which probes respond immediately to the presence of the proteins or nucleic acids in question. Compared to the two former technologies, chips of this kind promise to provide a real-time analysis of the concerned process (At-line analysis). Another advantage is the need for comparatively small amounts of samples.

The principle of chip-based measurements is presented, for example, diagrammatically in FIG. 2 of the article “Real-time electrochemical monitoring: toward green analytical chemistry” by J. Wang (Acc. Chem. Res.; ISSN 0001-4842; Rec. Sep. 12, 2001, pp. A-F). According to this, the sample to be analyzed is contacted with a biorecognition layer which may be, for example, an enzyme, an antibody, a receptor or DNA; the signal received thereby is emitted as voltage or electric potential via a transducer, for example an amperometric or potentiometric electrode, through an amplifier (amplification/processing). The study in question also mentions optical systems, compared to which the electronically analyzable systems were regarded by the author as being superior in regard to miniaturizability and other advantages.

Owing to the present invention, the protein-specific chips need not be considered. mRNA-recognizing chips are usually doped with complementary DNA molecules or DNA-analogs. Their manufacture and utilization for very detailed problems, such as for example the differentiation of point mutations, is described for example in the application WO 95/11995 A1. The DNA chip analyses include those with PCR amplification of the target sequence and those without amplification. There are also those with optical evaluation of the signals attributable to the recognition and those with electrical evaluation.

The optical detection methods partly require a mechanism for amplifying the signals. For this purpose, for example, fluorophores, acridinium esters or indirect detection via secondary binding events, for example via biotin, avidin/streptavidin or digoxigenin, have been described. In the last case, optical detection makes use of digoxigenin-specific antibodies that are labeled with an enzyme. Here, the enzyme activity is detected either colorimetrically or by way of luminescence. According to Westin et al. (2000), Nature Biotechnol., 18, pp. 199-204, hybridization may be coupled with a PCR on the DNA chip in order to be able to carry out the entire detection reaction on one chip (“lab-on-a-chip concept”).

Other studies have described the development of DNA chips that miniaturize the principle of capillary electrophoresis for DNA sequencing or separation (Woolley and Mathies (1994), Proc. Natl. Acad. Sci., 91, pp. 11348-11352; Liu et al. (2000), Proc. Natl. Acad. Sci. 97, pp. 5369-5374).

Electrically readable DNA chips have already been presented in principle in some publications (Hoheisel (1999), DECHEMA Jahresbericht 1999, pp. 8-11; Hintsche et al. (1997), EXS, 80, pp. 267-283). Wright et al. (2000; Anal. Biochem., 282, pp. 70-79) utilized an ion channel sensor (ICS) for DNA detection, as has been described for the first time by Cornell et al. (1997: Nature, 387, pp. 580-583). This is a process in which the conductivity of molecular ion channels is detected by means of a binding reaction. The sensor is essentially an impedance element. According to Cheng et al. (1998; Nat. Biotechnol, 16, pp. 541-546), it is possible to utilize electrical pulses for amplifying the hybridization reaction on optical DNA chips. Fritsche et al. (2002; Laborwelt II) proposed an electrical chip system which employs metallic nanoparticles bound to oligonucleotides, for example. In this system, “metallic amplification” during the hybridization reaction causes a decrease in the electrical resistance at the electrode, and can then be measured as a signal.

Another approach is based on an electrical detection principle which uses DNA probes that, due to labeling with a suitable enzyme (e.g. alkaline phosphatase), after hybridization result in an electrically active substrate that can then be detected via a redox reaction at the electrode (Hintsche et al. (1997), EXS, 80, pp. 267-283).

If in regard to the fundamental configuration and the evaluation system it is decided to use a particular nucleic acid-recognition chip type, the more specific problem arises as to which gene activities are to be observed. For technical reasons, the number of genes that can be analyzed simultaneously using one nucleic acid chip is limited. Thus, in regard to the number of probes that can be applied to the chip, optically readable chips are currently superior to those, which can be evaluated electrically. The limits of the latter chips are determined by the miniaturizability of the electronic measuring units.

Thus the biological problem arises, as to which gene activities suitably depict the concerned process. This also includes monitoring product formation, if, for example, said product is produced by fermentation. At the same time, control genes may also be included which indicate if the process develops in a direction, which is not intended. In the course of this monitoring, for reasons of practicability, the number of different genes observed should not be too high.

Biotechnological processes involving Gram-positive bacteria are of particular industrial interest. These bacteria, particularly owing to their secretion capability, are used for the industrial production of valuable substances. Among said bacteria, those of the genus Bacillus and among these in turn the species B. subtilis, B. amyloliquefaciens, B. agaradherens, B. licheniformis, B. lentus and B. globigii are currently economically the most important.

The studies presented below, for example, are concerned with the simultaneous observation of the activity of a plurality of genes in bacteria (multiparametric recording). The article “Monitoring of genes that respond to process-related stress in large-scale bioprocesses” by Schweder et al. (1999), Biotech. Bioeng., 65, pp. 151-159, describes the alteration in mRNA levels of various stress factor-inducible genes, namely clpB, dnaK (induced during heat shock), uspA (glucose deficiency), proU (osmotic stress), pfl and frd (O₂ deficiency) and ackA (glucose surplus) in the course of fermentation of E. coli and during the subsequent concentration phase. They were recorded by means of a PCR-based method carried out in a conventional manner. In this connection, different rates of expression were detected already at various sites in the reactor, as were responses to altered conditions, which took place in a matter of seconds.

Another fermentation of E. coli is described in the study “Monitoring of genes that respond to overproduction of an insoluble recombinant protein in Escherichia coli glucose-limited fed-batch fermentations” by Jürgen et al. (2000), Biotech. Bioeng., 70, pp. 217-224. Here, expression of the genes lon, dnaK, ibpB, htrA, ppiB, groEL, tig, s6, 19 and dps is observed partly at the mRNA level, partly at the protein level, partly at both levels. The investigation was carried out by means of 2D PAGE and the DNA array technique. In view of the results, it was suggested to monitor recombinant bioprocesses such as heterologous protein preparation via (directly) process-concerned proteins and reporter genes such as ibpB.

The study “Genomic analysis of high-cell-density recombinant Escherichia coli fermentation and “cell conditioning” for improved recombinant protein yield” by R. T. Gill et al. (2001; Biotech. Bioeng., 72, pp. 85-95) is concerned with another observation of the course of a fermentation in which a recombinant protein is expressed by E. coli. This study describes increased expression of the stress genes degP, uvrB, alpA, mltB, recA, ftsH, ibpA, aceA and groEL under the conditions mentioned with high cell density, compared to low cell density. Said genes were grouped among each other into certain clusters, according to the strength of the reaction. This was determined via an approach based on RT-PCR and DNA microarray, which was supplemented by dot blot analysis and which was applied to samples from two points in time of the fermentation, that is at the beginning, at low cell density, and towards the end, at high cell density. From this, cell conditioning approaches were developed in order to reduce the stress response of the cells.

Fundamental differences in the expression patterns of Gram-positive organisms compared to those of Gram-negative bacteria are reviewed by the study “Proteome and transcriptome based analysis of Bacillus subtilis cells overproducing an insoluble heterologous protein” by Jürgen et al. (2001), Appl. Microbiol. Biotechnol, 55, pp. 326-332. The study describes the expression inter alia of the genes dnaK, groEL, grpE, clpP, clpC, clpX, rpsB and rplJ in B. subtilis, as can be determined via the DNA macroarray technique and via two-dimensional polyacrylamide gel electrophoresis. According to this, the genes for purine synthesis and pyrimidine synthesis and those of particular ribosomal proteins are expressed in Gram-positive bacteria used for overexpression more strongly than was to be expected from the findings with Gram-negative bacteria. Another difference relates to the proteases Lon and Clp.

Since that time, several of these genes or even nucleic acid-binding chips with several of these genes have been disclosed, or at least the possibility of their manufacture revealed, in a plurality of publications. Thus, for example, the two patent applications DE 10136987 A1 and DE 10108841 A1 disclose in each case a gene from Corynebacterium glutamicum, namely clpC and citB, respectively. Both genes are described as being relevant for the amino acid metabolism, this being the reason for an intended, commercially interesting utilization of said genes, which comprises inactivating or at least attenuating said genes in order to optimize the fermentative production of amino acids by this microorganism. According to these applications, further possible applications may consist of providing probes for the gene products in question on nucleic acid-binding chips.

On the other hand, more genomic data of various organisms are increasingly published, which contain such an abundance of sequence data that a representative selection therefrom seems desirable. Thus the patent application WO 02/055655 A2 discloses more than 1800 DNA sequences, which have been determined by completely sequencing the genome of the microorganism Methylococcus capsulatus.

Since then, for example, the complete genome of the gram-positive Bacillus licheniformis has also been sequenced. It is described in the publication “The Complete Genome Sequence of Bacillus licheniformis DSM13, an Organism with Great Industrial Potential” (2004) by B. Veith et al. in J. Mol. Microbiol. Biotechnol., 7 (4), pp. 204 to 211, and in addition is available under the entry AE017333 (Bases 1 bis 4.222.645) in the databank GeneBank (National Center for Biotechnology Information NCBI, National Institutes of Health, Bethesda, Md., USA; http://www.ncbi.nlm.nih.gov; at the date of Feb. 12, 2004).

By using the technique of optically analysable chips it is now even possible to manufacture nucleic acid-binding chips that reveal an almost complete genome or the associated transcriptome (genomic DNA chips).

With the application WO 2004/027092 A2, a representative cross section with a manageable number of genes is made available so as to identify various physiological states that an observed microorganism can pass through in the course of cultivation. They include, for example, states of starvation relating to various nutrients or stress situations such as, for example, heat shock or cold shock, shearing stress, oxidative stress or oxygen limitation. It concerns the following genes. acoA, ahpC, ahpF, citB, clpC, clpP, codY, cspA, cspB, des, dnaK, eno, glnR, groEL, groL, gsiB, ibpA, ibpB, katA, katE, lctP, ldh, opuAB, phoA, phoD, pstS, purC, purN, pyrB, pyrP, sigB, tnrA, trxA and ydjF. The associated DNA sequences of B. subtilis, E. coli and/or B. licheniformis are also found in this application. This also permitted the corresponding nucleic acid-binding chips to be manufactured, which when monitoring a bioprocess based on microorganisms, particularly for Gram-positive or Gram-negative bacteria, indicate changes in the metabolic activities characterizing said process.

Nucleic acid-binding chips that are based on this selection of genes furnish a certain, although overall rather only a rough outline of the concerned metabolic situations. Generally, they are not capable of specifically illuminating an individual partial problem; indeed a single positive signal can result from various situations or can also be a false positive, which is why—particularly so in such an ambiguous situation—it is sensible to separately analyze a chosen metabolic aspect. Indeed, for electrically readable nucleic acid-binding chips that have the advantage of a real-time analysis, the number of simultaneously available sites is limited, such that it is not simply additional gene probes that can be added for the analysis of specific metabolic situations.

Nucleic acid-binding chips for monitoring bioprocesses that are specifically focussed on the detection of phosphate deficiency situations or glucose deficiency situations are disclosed in the not previously published applications DE 102004061664.7 and DE 102005022145.9. They each carry a limited number of probes to indicate these specific stress situations.

A metabolic situation that can be critical for microorganisms and consequently limiting for a corresponding bioprocess, is that of nitrogen deficiency. Nitrogen is an essential element for nucleic acids and for amino acids and consequently for proteins, which themselves represent the major functional carriers and nutrients of biological organisms. Nitrogen deficiency therefore stands for a deficient provision of nutrient and can rapidly lead to the dying off of the cells or can at least prevent the achievement of higher cell densities. A nitrogen deficiency also affects the rate of product formation, especially during the fermentative production of proteins, such as for example industrial enzymes. Consequently, there exists a particular need in this regard to carry out a chip-based real-time analysis and because of the thereby rapidly obtained results, to be able to selectively and therefore more purposefully engage in the ongoing bioprocess. In this way a loss of product, which would result from an unrecognised or tardily recognized nitrogen shortage, is prevented.

Accordingly, the object was to identify genes that in organisms, especially microorganisms, can be associated as unambiguously as possible with the stress signal of nitrogen deficiency. The aim was to develop probes for these genes in order to be able to employ said probes to monitor corresponding bioprocesses.

Consequently, it should be possible to occupy nucleic acid-binding chips with gene probes for single or a plurality of these genes and thereby arrive at nucleic acid-binding chips that dependably notify the signal “nitrogen deficiency” in the course of a monitored bioprocess (nitrogen deficiency sensors). This object posed itself especially for those nucleic acid-binding chips, whose number of occupiable sites, due to their type of construction, is comparatively small, especially the electrically readable chips. Having said that, these chips have the advantages of a rapid readability and therefore permit an At-line analysis. This guarantees an if needed early intervention to optimise the bioprocess in question in regard to the nutrient supply.

Such a DNA-binding chip should be applicable in a plurality of comparable processes and be customized with comparatively minor variations for specific application possibilities. It should preferably be directed to bioprocesses based on Bacillus species, particularly B. subtilis, B. amyloliquefaciens, B. lentus, B. globigii, and quite particularly on B. Licheniformis. Among bioprocesses, special emphasis was on fermentations, in particular the industrial manufacture of products, quite particularly of overexpressed proteins.

A nitrogen deficiency sensor of this kind should also make possible corresponding processes for measuring the physiological state of the concerned cells and also corresponding possible uses for monitoring the concerned biological processes.

To achieve this object, a large number of genes from the biologically important bacterium B. licheniformis was investigated (Example 1) in regard to their activatability by converting the concerned culture into a state of nitrogen deficiency. Here it was surprisingly determined that by far not all genes involved in nitrogen metabolism deliver a well defined signal in this regard. In addition, likewise surprisingly, there was observed an activation of such genes that had not been brought into contact with the nitrogen metabolism, for example the gene for a putative malate synthesis, or those that code for proteins with a still unknown function. Furthermore, those genes were discounted from among these, which clearly respond to more than only this one stress signal. The genes identified in this way should now be considered, if required independently from their previously known function, according to the invention as nitrogen metabolic genes. This is supported by Example 2 of the present invention. Inductions of very different strengths were observed in this case. According to the teaching of the present invention, genes should be all the more suitable as indicators, the stronger this response. According to the invention, those genes are therefore selected as the nitrogen deficiency indicators, which provide a clear signal that is significantly above a defined threshold value. The more the result lies above this limit, the more they are to be regarded as a part of the solution of the inventive problem, and makes clear the corresponding grading of the preferred inventive aspects (see below).

In Table 1 are shown all 210 genes from Bacillus licheniformis DSM13 that were investigated in Example 1, whose induction under nitrogen deficiency was observed, wherein a factor of at least three was regarded as being significant. From these, all 49 genes shown in Table 2 are those whose induction by nitrogen deficiency amounted to at least the factor 8 at any of the measured times and for which it could be concluded (from experiments not shown here) that they were comparatively specific for this signal. In Table 3 they are again listed with respect to the strength of their observed maximum induction. Their DNA and amino acid sequences are listed in the sequence listing of the present application, wherein the odd numbers stand for DNA sequences and the following even numbers stand respectively for the derived amino acid sequences. The concerned SEQ ID numbers in Tables 2 and 3 also refer to these sequences. The following genes are concerned in the order of decreasing strength of the induction initiated by nitrogen deficiency (see Tables 2 and 3):
gene (homolog to SEQ ID NO. 81, 82) coding for a hypothetical protein of unknown function;
yrkC (unknown function—similar to proteins of unknown function; SEQ ID NO. 83, 84);
nasF (uroporphyrine-III C-methyltransferase; SEQ ID NO. 39, 40);
gene (homolog to SEQ ID NO. 93, 94) coding for a putative protein (putative nitrogen regulation protein P-II);
pckA (phosphoenol pyruvate-carboxykinase; SEQ ID NO. 43, 44);
trpE (anthranilate-synthase; SEQ ID NO. 67, 68);
nasB (electron-transfer-subunit of the assimilatory nitrate reductase; SEQ ID NO. 33, 34);
gene (homolog to SEQ ID NO. 15, 16) coding for a hypothetical protein (close homolog to the aldehyde-dehydrogenase DhaS);
nasC (catalytic subunit of the assimilatory nitrate reductase; SEQ ID NO. 35, 36);
ycdH (unknown function—similar to the binding protein of the ABC transporter; SEQ ID NO. 65, 66);
gene (homolog to SEQ ID NO. 63, 64) coding for a putative protein (putative ammonium transporter);
nasD (sub unit of the assimilatory nitrite reductase; SEQ ID NO. 37, 38);
gene (homolog to SEQ ID NO. 95, 96) coding for a hypothetical protein of unknown function;
gene (homolog to SEQ ID NO. 49, 50) coding for a putative protein (putative Na(+) bonded D-alanine-glycine permease);
gene (homolog to SEQ ID NO. 45, 46) coding for a putative protein (putative malate synthase);
gene (homolog to SEQ ID NO. 85, 86) coding for a putative protein (putative glycosyl hydrolase/lysozyme);
gene (homolog to SEQ ID NO. 9, 10) coding for a putative protein (putative serine protease);
gene (homolog to SEQ ID NO. 17, 18) coding for a putative protein (putative transcription regulator);
trpC (indole-3-glycerine phosphate synthase; SEQ ID NO. 71, 72);
trpB (beta-sub unit of tryptophan synthase; SEQ ID NO. 75, 76);
ycnK (unknown function—similar to the transcription regulator of the DeoR family; SEQ ID NO. 31, 32);
gene (homolog to SEQ ID NO. 21, 22) coding for a putative protein (putative phage capsid protein);
trpD (anthranilate-phosphoribosyl transferase; SEQ ID NO. 69, 70);
ydfS (unknown function—similar to proteins of unknown function; SEQ ID NO. 79, 80);
trpF (phosphoribosyl-anthranilate isomerase; SEQ ID NO. 73, 74);
gene (homolog to SEQ ID NO. 47, 48) coding for a putative protein (putative hydrolase);
yvlB (unknown function—similar to proteins of unknown function; SEQ ID NO. 89, 90);
yncE (unknown function; SEQ ID NO. 87, 88);
yvlA (unknown function; SEQ ID NO. 97, 98);
glnR (transcription repressor of the glutamine synthetase gene; SEQ ID NO. 11, 12);
ycnJ (unknown function—similar to the copper export protein; SEQ ID NO. 29, 30);
gene (homolog to SEQ ID NO. 55, 56) coding for a putative protein (ATP binding protein of a putative ABC transporter);
gene (homolog to SEQ ID NO. 19, 20) coding for a conserved hypothetical protein of unknown function;
nrgB (nitrogen regulating PII-similar protein; SEQ ID NO. 3, 4);
yvtA (unknown function—similar to the HtrA-similar serine protease; SEQ ID NO. 23, 24);
yciC (unknown function—similar to proteins of unknown function; SEQ ID NO. 57, 58);
nrgA (ammonium transporter; SEQ ID NO. 5, 6);
glnA (glutamine synthetase; SEQ ID NO. 13, 14);
alsR (transcription regulator of the alpha-acetolactate operon; SEQ ID NO. 61, 62);
ggt (gamma-glutamyl peptidase; SEQ ID NO. 51, 52);
yqjN (unknown function—similar to the amino acid decomposition; SEQ ID NO. 1, 2);
yppF (unknown function; SEQ ID NO. 77, 78);
gene (homolog to SEQ ID NO. 53, 54) coding for a hypothetical protein of unknown function;
gene (homolog to SEQ ID NO. 41, 42) coding for a hypothetical protein of unknown function;
ycnl (unknown function—similar to proteins of unknown function; SEQ ID NO. 27, 28);
htrA (serine protease Do; Heat shock protein; SEQ ID NO. 7, 8);
gene (homolog to SEQ ID NO. 91, 92) coding for a putative protein (putative ABC transporter/amino acid permease);
citA (auxiliary citrate synthase I; SEQ ID NO. 59, 60);
kdgR (transcription repressor of the pectin decomposition operon; SEQ ID NO. 25, 26).

From further experiments (see Example 5) it was determined that the following gene is also inventively suitable as an indicator:
tnrA (a global, transcription regulator involved in the nitrogen metabolic regulation; SEQ ID NO. 99, 100).

A solution to the posed problem therefore consists of a nucleic acid-binding chip provided with probes for at least three of the following 50 genes: kdgR, citA, gene coding for a putative protein (putative ABC transporter/amino acid permease) (homolog to SEQ ID NO. 91), htrA, ycnl, gene coding for a hypothetical protein of unknown function (homolog to SEQ ID NO. 41), gene coding for a hypothetical protein of unknown function (homolog to SEQ ID NO. 53), yppF, yqjN, ggt, alsR, glnA, nrgA, yciC, yvtA, nrgB, gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA,

wherein the total number of all the different probes specific for the nitrogen metabolism is not greater than 80.

More detailed information on these genes can be found in Examples 1 to 3 and the Tables 1 to 3 of the present application. These genes as obtainable from B. licheniformis DSM 13 are disclosed in the sequence listing. Most of them are also described from other species (see below). Some of them, however, concern putative (assumed/probable) genes or genes that code for putative (assumed/probable) enzymes. According to the invention, these are defined as far as possible, based on databank comparisons, as those with a putative (assumed) function and in addition as homologs to the found B. licheniformis genes. On this, two different points must be explained:

Firstly, for one or others, it could emerge from a biochemical analysis that these putative functions are not consistent with the real function. In this case the putative function will not be retained. In fact, these results do not challenge the invention in so far as according to the invention, it solely comes down to the observation of increased transcription in connection with the nitrogen deficiency, such that the gene activity in question, independently of the initially performed enzyme activity, can for all intents and purposes serve as the indicator for nitrogen deficiency.

secondly, in the absence of a gene name, no matching definition can be found for this other than that about the gene itself. One therefore speaks of homologs. Thus one assumes that in species other than B. Licheniformis under nitrogen deficiency the homologous genes are activated. Should it turn out that in a species under consideration a plurality of homologs exists to one of these genes, and said homologs are capable of transcript formation in vivo, then the indication “homolog to SEQ ID NO. . . . ) refers to the closest similarity of each of the various genes under consideration.

According to the invention, at least three of these genes are selected so as to obtain the conclusion with the highest possible reliability, i.e. in order to eliminate a single false positive signal traced back to only one probe type.

According to the invention, a nucleic acid-binding chip is understood to mean all objects that are provided with nucleic acid-specific probes and when binding to one or more specifically recognized nucleic acids deliver an analysable signal.

The composition of chips that are doped with nucleic acids as probes is known from the prior art presented in the introduction. In principle they can all be used for embodiments of the present invention. They are based on the principle of the nucleic acid hybridisation of the mRNA to be detected (or a molecule derived therefrom) with the probe supplied on the chip. According to the system for evaluating the signal initiated by the hybridisation, one distinguishes between chips having an optical and an electrical analysis system. According to the invention, both systems can be used in principle.

Such chips are employed as follows for monitoring each of the bioprocesses under consideration: At a defined time a sample with the biological material to be analysed is taken from the process. RNA, especially mRNA, is isolated from the sample by known methods, for example by cell digestion and the use of a denaturing buffer. The RNA is self tagged or used as the starting molecule for a molecule introduced into the measurement (for example cDNA obtained by reverse transcription) and the resulting molecules in a buffer are preferably conducted over/through the chip. During hybridisation (sandwich marking) of a prepared RNA or its derivative with the homologous (i.e. congruent with respect to its sequence) probes provided on the chip (target nucleic acid, for example target DNA or target nucleic acid analog) there results a corresponding optically or electronically analysable signal. This is based for example on the marking of the binding mRNA or a transcript thereof with a color marker or fluorescent marker, a hybridisation with a second probe or on a secondary detection reaction, for example via a RT-PCR.

As at any one time a plurality of molecules is generally bound to the chip from the same probe, the strength of the hybridisation signal over a given—in the individual case to be optimised if needed—area is proportional to the number of specific mRNA present in the sample at the time of sampling. In this way the strength of the signal is a direct measure of the activity of the gene in question at the time of sampling.

The time span between sampling and measurement should be as short as possible, for example by a largely automated sampling, its preparation and passage over/through the sensor.

In principle, all plants, animals and microorganisms, especially those that are commercially utilized, can be considered as the organisms monitored using an inventive chip. From the application DE 19860313 A1 entitled “Process for the recognition and characterization of agents against plant pathogens”, it emerges that metabolic situations must be observed in plants, particularly useful plants. Similarly, livestock or laboratory animals, for example can also be observed. Eukaryotic cell cultures are of quite considerable commercial interest, for example for the production of monoclonal antibodies, and in particular fermentative production of food, for example via alcoholic fermentation carried out by yeasts. Bacteria are utilized, in particular, for industrial production of proteins or low molecular weight desired substances (biotransformation), for example vitamins or antibiotics.

According to the invention, probes are understood to mean all molecules that are capable of incurring an essentially specific interaction with nucleic acids (to bind them). According to the invention, this interaction is exploited in order to obtain, in the context of a suitable configuration (chip), a largely unambiguously assignable and analysable signal.

From the chemical point of view, an inventive probe is a mostly a compound which is capable of binding mRNA molecules or nucleic acids derived therefrom through hydrogen bonds, as is the case, for example, for the interaction of the two strands of a DNA or for DNA-RNA interaction. For example, this can be a DNA, which is more stable to hydrolysis than RNA.

In addition, further molecules are known in the prior art, in particular chemically synthesized ones, which biomimetically make possible the same interaction but are more stable than DNA, for example by exchanging the phosphate ester bonds of the backbone against less hydrolysis-sensitive bonds. Such nucleic acid-analog probes characterize preferred embodiments of the present application (see below). The corresponding specific probes would have to be synthesized according to the example of the sequence listing related to this application. Advantageously, it should be possible to use chips of the invention multiple times, in particular during a single observed process in the course of which continuous monitoring is desirable.

Limiting the usability of a probe is thus in each case the extent of homology between the probe provided and the mRNA or the nucleic acid derived therefrom respectively, which is to be recognized by way of hybridization. Ultimately, the extent of hybridization of the probe with the mRNA to be detected (see above) decides its usability as a probe and, in individual cases, has to be optimized experimentally and/or taken into account by way of adjusting signal evaluation. Under the conditions determined by the construction of the measuring apparatus and other influences, a hybridization must take place which can specifically be attributed only to the gene of interest, is sufficiently strong in order to give a positive signal and, on the other hand, is not too strong such that, after the signal has been generated, the mRNA diffuses off in order to render the binding site empty for the next molecule, or to enable the signal to decay; the last, if needed in the course of a suitable washing step.

It is, however, necessary to estimate the extent of homology between the genes in question prior to using inventive chips for an organism of interest; if the affinity of the mRNAs to be detected to the initially introduced probes is not sufficient, to anchor those probes by way of the same genes of the invention from closer related species on the chip and to carry out calibration measurements in order to obtain reliable information as to which signal strength corresponds to which concentration of special mRNA.

The identification of the important 50 genes for the present invention is described in the examples of the present application. Their sequences obtained from B. licheniformis are presented in the sequence listing of the present application (SEQ ID NO. 1 to 100), wherein the sequences with odd numbers concern DNA sequences and each sequence with one higher number concern the amino acid sequences derived therefrom. Whereas the DNA sequences can be directly used for manufacturing probes (see above), the amino acid sequences serve for example to verify the gene function using sequence databank comparisons and moreover can be used to generate similar nucleic acid recognizing probes using back translations of the genetic code.

As is illustrated in example 1, numerous different gene transcripts, i.e. mRNA molecules were investigated, especially those that were known to participate in the nitrogen metabolism. These mRNA molecules were isolated at various times during the transition of B. licheniformis DSM 13 to a state of nitrogen deficiency. Example 1 likewise describes the way in which the increase in concentration of said mRNA inside B. licheniformis cells was determined experimentally. Possible alternative determinations of this may be established in the prior art; the compilation in Table 1 (Example 2) is critical to the understanding of the present invention. It depicts the changes in the concentrations of a total of 210 mRNAs linked to the transition. In this connection, the following thresholds of the ratio of the amount of RNA of the particular gene to the control value were considered significant: according to the invention, the genes whose RNA has a ratio of >3 (i.e. at least a three-fold increase) are considered induced; a distinct induction is present at a ratio of >10; genes having an RNA ratio of <0.3 (i.e. a decrease to less than 30%) are distinctly repressed. For the 210 genes listed in Table 1, at least a three-fold increase was observed at any of the observed times.

As is proven in Example 3, among these 210 genes there are only 67 genes having an at least 8 fold induction at any of the observed times under the conditions of nitrogen deficiency described in Example 1. Among these, 18 can be regarded as being not particularly specific, such that surprisingly solely the 49 genes listed in Table 2 remain as specifically distinctly induced genes under nitrogen deficiency. According to the invention, these 49 genes are considered representative indicators of a nitrogen deficiency condition. Further information about these genes, for example about their function or deviating start codons, can be found in Tables 2 and 3 and in the sequence listing; the particular English names for the corresponding proteins have already been listed above, in the sequence of data presented in Table 3.

According to Example 5, the separately identified gene tnrA shows a more than 15 fold induction 30 minutes after the transition into the stationary phase induced by nitrogen deficiency, such that this gene can also be utilized for the realization of the inventive teaching and is correspondingly preferred.

All these genes have been described in each case individually in the prior art. They can be found for the various organisms in generally accessible databases. As mentioned above, the sequences indicated in the sequence listing for B. licheniformis DSM 13 have been determined from this microorganism and are virtually identical to the information described in the publication “The Complete Genome Sequence of Bacillus licheniformis DSM13, an Organism with Great Industrial Potential” (2004) by B. Veith et al. in J. Mol. Microbiol. Biotechnol., Volume 7(4), pages 204 to 211 and additionally accessible under the entry AE017333 (bases 1 to 4 222 645) in the GenBank database (see above). The B. licheniformis DSM 13 strain is generally obtainable in the Deutsche Sammlung von Mikroorganismen and Zellkulturen GmbH, Mascheroder Weg 1b, 38124 Braunschweig, Germany (htt://www.dsmz.de). It has the deposition number ATCC 14580 at the American Type Culture Collection, 10801 University Boulevard, Manassas, Va. 20110-2209, USA (http;//www.atcc.org).

A large proportion of the genes from other organisms, which correspond to the 50 genes mentioned, have likewise been deposited in generally accessible databases, for example for the well-characterized species B. subtilis and E. coli which are generally regarded as model organisms of Gram-positive and Gram-negative bacteria, respectively. The corresponding sequences may be found, for example, in the databases of Institut Pasteur, 25, 28 rue du Docteur Roux, 75724 Paris CEDEX 15, France, which are accessible via the internet addresses http://genolist.pasteur.fr/Colibri/ (for E. coli) and http://genolist.pasteur.fr/Colibri/ (for B. subtilis), respectively (as of 12.2.2004). Other databases suitable for this are that of the EMBL-European Bioinformatics Institute (EBI) in Cambridge, United Kingdom (http://www.ebi.ac.uk), Swiss-Prot (Geneva Bioinformatics (GeneBio) S.A., Geneva, Switzerland (http://www.genebio.com/sprot.html) or GenBank (National Center for Biotechnology Information NCBI, National Institutes of Health, Bethesda, Md., USA).

Said “corresponding genes” mean those which in each case code for the proteins which catalyze the same chemical reaction in the observed organism or which are involved in the same physiological process as the cited 50 proteins in B. licheniformis DSM 13. For other organisms, most of these have similar names and abbreviations to those indicated for B. licheniformis in Tables 1, 2 and 3 because these names represent the particular function. If in doubt, they can be recognized via their sequence, which in each case is the next similar (most homologous) for the organism in question to the sequences mentioned herein. When assigning the function, the similarity of the amino acid sequences to one another is especially decisive because the amino acids are the carriers of the function of the protein and, owing to the degeneracy of the genetic code, various nucleotide sequences can code for the same amino acid sequence.

Particularly high degrees of relationship exist between closely related species. Thus it can be assumed in principle that for most of the cited 50 genes, homologs can be found in all species, even in cyanobacteria, in eukaryotic cells such as, for example, fungi, or in Gram-negative species such as E. coli or Klebsiella. This probability is even higher for Gram-positive bacteria, in particular of the genus Bacillus, because B. licheniformis DSM 13, from which the sequences listed in the sequence listing are derived, is such a Gram-positive bacterium. It can moreover be assumed that in increasingly related organisms the homologous genes are also increasingly subjected to the same regulatory mechanisms or regulatory mechanisms acting in the same way; therefore, these homologs should also indicate the same metabolic situation, in particular a nitrogen deficiency. In this respect, B. licheniformis is a good choice of an exemplary organism because the commercially likewise particularly important species B. subtilis, B. amyloliquefaciens, B. lentus, B. globigii are likewise Bacilli and therefore Gram-positive. This is in accordance with the concerned aspect of the stated object.

It should be noted that, in order to practice the invention for a particular species, not all of the 50 mentioned genes must be known but that only a few of them (see below) are sufficient in order to reproduce nitrogen metabolism and, in particular, to be able to detect the transition to a nitrogen deficiency condition. Nevertheless, the reliability of the information about the nitrogen supply state increases with an increasing number of probes. If a plurality of genes are known that on the basis of the present disclosure appear to be suitable in principle, it is recommended to carry out an expression study prior to preparing a corresponding chip, in order to check whether the genes in question actually allow significant information, similarly to the representation in Example 1 or else via a Northern analysis. Shifts regarding the level of expression caused by nitrogen deficiency may be more likely the less related the observed species is to B. licheniformis, such that subgroups (where appropriate other subgroups than the ones listed below) of these 50 genes prove to be particularly suitable and therefore preferred.

Accordingly, the preparation of an inventive nucleic acid-binding chip for an organism not specified herein must involve the identification of the homologous genes that correspond to at least some of the genes specified for B. licheniformis, for example by comparing the known DNA sequences of the organism in question with the sequences indicated herein. These or parts thereof (see below) may then serve per se as probes or as a template for synthesizing corresponding probes that are applied to a nucleic acid-binding chip by methods known per se.

If it is the case that some homologous sequences have not been deposited in databases, it is possible for the person skilled in the art to synthesize particular probes on the basis of the sequences disclosed in the sequence listing of the present application in order to screen with the aid thereof a gene library generated for the desired organism (genomic or preferably on the basis of the cDNA) for the homolog in question by generally standard methods. As an alternative to this, it is also possible, on the basis of the DNA sequences indicated in the sequence listing, to synthesize oligonucleotides that serve as PCR primers so as to amplify the genes in question or parts thereof that can be used as probes from a DNA preparation of the complete genome or from a cDNA preparation of the organism of interest. These or parts thereof (see below) can be employed as probes on inventive nucleic acid-specific chips.

An essential feature of the present invention is the fact that the total number of all the different nitrogen metabolism-specific probes does not exceed 80. This feature correlates with the stated object, according to which it should mainly focus on those nucleic acid-binding chips whose number of occupiable places is comparatively low due to their type of construction. In particular, these are the electrically analyzable chips.

Further nitrogen metabolism-specific probes may include, for example, those which are induced by an excess of nitrogen, and may include also others which appear not to be directly associated with nitrogen metabolism, but which may be defined as such due to said inducibility. As a result, such a chip also produces analyzable information useful in the observed process, after nitrogen deficiency has been overcome, for example by carrying out appropriate countermeasures.

Furthermore, nucleic acid-specific probes are usually in each case only fragments of the complete genes (see below). In individual cases, for example with regulation via splicing or with large, multifunctional polypeptides, it may therefore be sensible to detect the same gene with two or more different probes. Corresponding embodiments are therefore, where appropriate, characterized by more than 50 probes which, however, do not respond to more than said 50 genes.

Depending on the process to be observed, probes for additional genes or gene products may also be comprised on the inventive chips (see below).

On the other hand, the core of the invention consists specifically of the specificity of the chip in question, with which a special metabolic situation is to be recorded. In the case of such a specific question, the preparation of a chip with more than 80 probes responding to various genes, or even of a chip that reproduces a large part of the genome of an organism, is not part of the invention described herein, due to the complexity associated therewith. Rather, both kinds of chips can be sensibly used in parallel in an observed bioprocess: thus the chips containing a number of various gene probes or containing a representative cross section of various, possibly concerned situations, as are provided by the application WO 2004/027092 A2, can provide a rough overview of the condition of the organism in question, whereas a chip of the invention is used as a control, should there be reason to be concerned that the cells in question could enter a nitrogen deficiency state.

A preferred embodiment is an inventive nucleic acid-binding chip that is doped increasingly preferably with the probes indicated above in the order indicated there.

This means the genes listed in Table 3 in reverse order. Accordingly, chips with probes for the kdgR gene (transcription repressor of the pectin break down operon; SEQ ID No. 25, 26) are least preferred among the inventive chips with respect to gene selection for the formation of probes, since this gene has the weakest induction among the mentioned 50 genes, whose transcription is enhanced in a significant manner due to nitrogen deficiency. In contrast, those chips with a probe for the gene that codes for a hypothetical protein of unknown function; (homologous to SEQ ID No. 81, 82) are most preferred in regard to gene selection. This is because this gene showed a more than 146 fold induction over the starting level and therefore has the highest induction of all the genes measured. The signal linked thereto should thus be the most suitable of all the investigated genes for indicating the metabolic situation “nitrogen deficiency”.

According to Example 5, tnrA is between trpC and trpB and is correspondingly preferred.

A preferred embodiment is an inventive nucleic acid-binding chip, wherein at least one, increasingly preferably two or three of the probes are selected from the following 34 genes:

gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA,

In the studies carried out in the examples on the basis of B. licheniformis DSM 13 and depicted in Examples 1 to 3 and 5, these genes exhibited gene inductions that were elevated by at least a factor of 10. Correspondingly preferred embodiments are thus characterized by the first 33 of the genes listed in Table 3.

Further preferred are those inventive nucleic acid-binding chips, wherein at least one, increasingly preferably two or three of the probes are selected from the following 20 genes:

trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA,

In the studies carried out in the examples on the basis of B. licheniformis DSM 13 and depicted in Examples 1 to 3 and 5, these genes exhibited gene inductions that were elevated by at least a factor of 15.

Further preferred are those inventive nucleic acid-binding chips, wherein at least one, increasingly preferably two or three of the probes are selected from the following 12 genes:

nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81),

In the studies carried out in the examples on the basis of B. licheniformis DSM 13 and depicted in Examples 1 to 3, these genes exhibited gene inductions that were elevated by at least a factor of 20.

Further preferred are those inventive nucleic acid-binding chips, wherein at least one, increasingly preferably two or three of the probes are selected from the following 4 genes:

gene coding for a putative protein (putative nitrogen regulation protein P-II) (homolog to SEQ ID NO. 93), nasF, yrkC, gene coding for a hypothetical protein of unknown function (homolog to SEQ ID NO. 81).

In the studies carried out in the examples on the basis of B. licheniformis DSM 13 and depicted in Examples 1 to 3, these genes exhibited gene inductions which were elevated by at least a factor of 30, in the case of yrkC by more than 60 and in the last case even significantly more than a factor of 100.

In preferred embodiments, nucleic acid-binding chips of the invention are doped with at least 4, 5, 6, 7, 8, 9, 10, 12, 14, 16, 18, 20, 22, 24, 26, 28, 30, 35, 40, 45 or 50 of the probes specified for the present invention.

The more of these probes that respond to a corresponding signal, the more reliable is the information related thereto about the supply or inadequate supply of nitrogen at that moment. Thus it is also sensible to combine the particularly meaningful probes which therefore characterize preferred embodiments with apparently less meaningful ones in order to be able to rule out false-positive signals. A further advantage consists of combining, according to the information in Table 2, those probes with one another that produce signals of different strength at different times indicated there. Thus it is possible to estimate the time interval from the onset of nitrogen deficiency to the sampling and the possibility of said deficiency being attributed to a particular environmental influence—while recording the culturing conditions.

With respect to their specific sequences, various probes which, however, respond to the same mRNA, for example fragments which hybridize with different regions of the same mRNA (see below), may be present several times in order to increase the read-out reliability, but they are only counted once for the purposes of this aspect of the invention, since they respond, in the best case equally strongly to the same signal and would in principle be exchangeable with one another.

In preferred embodiments of nucleic acid-binding chips of the invention, the total number of all the different probes does not exceed, in increasing preference 80, 75, 70, 65, 60, 55, 50, 40, 30, 20 or 10.

This corresponds to the inventive concept stated above, according to which the chips described herein are intended to monitor a special metabolic aspect so that it is not necessary to apply a larger number of probes to the chips in question than is required for recording this situation. This does not affect the situation in which it may be sensible in individual cases to employ more than one probe for detecting the same mRNA and/or to apply individual probes that are linked to the production of a valuable substance of interest. Overall, the present invention moves within the specified scope in order to be able to include within the scope of protection chips that for technical reasons have only a few binding sites.

In preferred embodiments of nucleic acid-binding chips of the invention, the probes referred to as being relevant to the invention are those which react to the concerned, respectively most homologous, in vivo transcribable genes from the organism chosen for the bioprocess, preferably those which are derived from the concerned, respectively most homologous, in vivo-transcribable genes of this same organism.

To this end, it has already been stated above that the sequences disclosed in the present application have been obtained from B. licheniformis and should be suitable particularly for monitoring related species, in particular those of the genus Bacillus, owing to the generally known relationships. This applies both to the identified genes to which it was possible to assign specific functions based on database comparisons and which should in principle encode the same function in other species, and to those which have been defined only by their sequences. To this end, the most closely related genes in the observed organism are used according to the invention for deriving corresponding probes. However, care must be taken here that probes are generated not for pseudo genes but for those that are actually transcribed to mRNA under in vivo conditions, i.e. which result in a nucleic acid signal that can be measured in the cytoplasm.

From a statistical point of view, however, such a chip should be all the more successfully usable, the better is the interaction of the chosen probes with the nucleic acids being measured. As a result, especially with a decreasing degree of relationship to B. licheniformis, it becomes increasingly necessary not to rely on the indicated sequences for this hybridization, but—if sequence differences exist—to use those for the homologous genes from the species in question. The latter sequences can, as explained above, be obtained by methods per se, in particular gene library screening or PCR with primers based on the sequences disclosed herein (where appropriate in the form of “mismatch primers” containing certain, random sequence variations).

In preferred embodiments of nucleic acid-binding chips of the invention, the organism selected for the bioprocess is a representative of unicellular eukaryotes, Gram-positive or Gram-negative bacteria.

This is because these groups include the commercially most employed organisms, in particular if the bioprocess to be observed is a fermentation. This includes, for example, fermentative processes, for example for producing wine or beer, or biotechnological production of valuable substances such as proteins or low molecular-weight compounds.

Various organisms are chosen for a biotechnological method, depending on the type of the desired product. In the context of the invention, this means not only the producer strains but also any organisms upstream of the production process, for example for the cloning of corresponding genes or the selection of suitable expression vectors. In this connection, the need for recording nitrogen limitation is in principle present during each part of the process.

In preferred embodiments of nucleic acid-binding chips of the invention, the unicellular eukaryotes are protozoa or fungi, among these in particular yeast, quite particularly Saccharomyces or Schizosaccharomyces.

This is because the latter are employed intensively as host cells, in particular for the gene products of eukaryotes, in addition to being employed for producing alcoholic beverages and other food obtained by fermentation. The former usage is particularly advantageous, if said gene products are to undergo special modifications that can only be carried out by these strains, such as glycosylations of proteins, for example.

This subject matter also includes inventive chips that are geared to monitor the course, in particular the growth, of cell cultures of higher eukaryotes, for example of rodents or humans. In a certain sense, they may likewise be understood as meaning, at least substantially, unicellular eukaryotes that are of considerable commercial importance, in particular in immunology, for example for producing monoclonal antibodies.

In preferred embodiments of nucleic acid-binding chips of the invention, the Gram-positive bacteria are Coryneform bacteria or those of the genera Staphylococcus, Corynebacteria or Bacillus, in particular of the species Staphylococcus carnosus, Corynebacterium glutamicum, Bacillus subtilis, B. licheniformis, B. amyloliquefaciens, B. agaradherens, B. stearothermophilus, B. globigii or B. lentus, and quite particularly B. licheniformis.

This is because they are industrially particularly important producer strains. They are employed in particular for producing low molecular weight chemical compounds, for example vitamins or antibiotics, or for producing proteins, in particular enzymes. Particular mention must be made here of amylases, cellulases, lipases, oxidoreductases and proteases. The particular orientation toward B. licheniforms can be explained by the fact that the sequences indicated in the sequence listing have been obtained from this species and that, as described in Examples 2 and 3, it was possible to prove their connection with the transition to nitrogen deficiency.

In no less preferred embodiments of nucleic acid-binding chips of the invention, the Gram-negative bacteria are those of the genera E. coli and Klebsiella, in particular derivatives of Escherichia coli K1 2, of Escherichia coli B or Klebsiella planticola, and quite particularly derivatives of the strains Escherichia coli BL21 (DE3), E. coli RV308, E. coli DH5α, E. coli JM109, E. coli XL-1 or Klebsiella planticola (Rf).

This is because they are used for producing biological valuable substances, both on the laboratory scale, for example cloning and expression analysis, as well as on the industrial scale.

In preferred embodiments of nucleic acid-binding acids of the invention, at least one, increasingly preferably a plurality, of the probes specified in connection with the invention described herein is/are derived from the sequences listed in the sequence listing under numbers SEQ ID No. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97 and 99.

This is because it was possible to prove the connection of these genes with the transition to nitrogen deficiency in B. licheniformis, as described in Examples 2 and 3. These sequences should therefore be used in particular, if B. licheniformis or other, especially related, Bacillus species are to be monitored.

Preferred embodiments of nucleic acid-binding chips of the invention are those which are additionally doped with at least one probe for an additional gene, in particular one which is metabolically associated with the gene(s) additionally expressed depending on the process, very particularly for one of these or this one itself.

As explained above, the observed processes are of an industrial interest that is often related to further specific genes. This is, for example if a protein is to be produced, the gene for this protein and, if a low molecular-weight compound is to be produced, one or more gene products which are on the synthetic pathway of the compound in question or which regulate said pathway. Other genes intrinsic to the cell may also be affected, for example metabolic genes which must be increasingly produced in the course of product production, for example an oxidoreductase intrinsic to the cell, if the product is to be obtained from a reactant or an intermediate by oxidation or reduction.

Moreover, in certain biological processes, in particular the production of commercially relevant compounds by microorganisms, strains geared to the process in question rather than wild type strains are usually employed. They include, apart from transformation with the genes responsible for actual production of the product, provision with selection markers or further metabolic adjustments up to auxotrophies. Such strains have a particular profile of demands on the growth conditions, and some of them possess metabolic genes that are mutated from the wild type genes. Since chips of the invention are advantageously intended to be geared to exactly these strains, quite particularly to the observed bioprocess, these strain-specific peculiarities should be taken into account and may be reflected in the choice of the probes in question.

In preferred embodiments of such nucleic acid-binding chips of the invention, the gene additionally expressed depending on the process is that for a commercially usable protein, in particular an amylase, cellulose, lipase, oxidoreductase, a hemicellulase or protease, or one which is on a synthetic pathway for a low molecular-weight chemical compound or which at least partially regulates said pathway.

These are then geared particularly to those bioprocesses, especially fermentations, in which the cited proteins are produced. The latter are commercially particularly important enzymes that are used, for example, in the food industry or detergent industry. The latter case includes in particular the removal of soilings that are hydrolyzable by amylases, cellulases, lipases, hemicellulases and/or proteases, the treatment of the respective materials, in particular by cellulases, or the provision of an enzymatic bleaching system based on an oxidoreductase.

The latter variant falls within the area of biotransformation, according to which particular, where appropriate additionally introduced, metabolic activities of microorganisms are utilized for the synthesis of chemical compounds.

In preferred embodiments of nucleic acid-binding chips of the invention, one, preferably a plurality, of the probes specified in connection with the invention described herein is/are provided in single-stranded form, in the form of the codogenic strand.

This embodiment has the aim of improving hybridization between the probe and the sample to be detected. This applies in particular to the case in which the concerned mRNA content is actually determined from the sample. Since said mRNA is single-stranded and its sequence corresponds to the coding strand of the DNA, optimal hybridization with the complementary, i.e. codogenic, strand should ensue.

In preferred embodiments of nucleic acid-binding chips of the invention, one, preferably a plurality, of the probes specified in connection with the invention described herein is/are provided in the form of a DNA or of a nucleic acid analog, preferably in the form of a nucleic acid analog.

This embodiment has the aim of improving the durability and multiple usability of the chips of the invention. This need arises in particular during a single observed process in the course of which constant monitoring is desirable. The durability of chips of the invention, in particular toward nucleic acid-hydrolyzing enzymes, is already increased by providing the probes in the form of a DNA, since the latter is hydrolytically less sensitive per se than an RNA, for example. Even more stable are nucleic acid analogs in which, for example, the phosphate of the sugar-phosphate backbone has been replaced with a chemically different building block that cannot be hydrolyzed, for example, by natural nucleases. Such compounds are known in principle in the prior art and are commercially synthesized by specialized companies for customer indicated sequences. The probes in question can be synthesized, for example, according to the template of the sequences indicated in the sequence listing.

In preferred embodiments of nucleic acid-binding chips of the invention, one, preferably a plurality of the probes referred to as being relevant to the invention comprises/comprise gene regions that are transcribed into mRNA by the organism to be studied, in particular the gene regions close to the 5′ end of said mRNA.

This takes into account the aspect that in many cases the regulatory DNA sections are also assigned to a special gene. However, the chip of the invention is actually intended to be employed for detecting the mRNA actually present in the observed cells, such that, for the purpose contemplated herein, only the gene section that is actually translated into mRNA is important. Secondly, it must also be considered that introns occur, in particular in eukaryotes, i.e. the coding region is interrupted by sections which are not translated into mRNA. Probes that contain introns should therefore respond to the mRNA in question only poorly or not at all. To implement this aspect, it is advisable to use cDNA sequences rather than genomic DNA sequences, i.e. those sequences that have been obtained on the basis of the actual mRNA.

Furthermore, detection of an mRNA often does not require hybridization over the entire length of the sequence. The specific probes therefore need to comprise usually only a smaller part of the gene transcribed to mRNA. Advantageous for this is a selection of a region close to the 5′ end of the mRNA, since this region is the first to be transcribed to mRNA and therefore the first to be detectable after activation of the gene. This benefits real-time detection.

In preferred embodiments of nucleic acid-binding chips of the invention, one, preferably a plurality, of the probes referred to as being relevant to the invention reacts/react to fragments of the concerned nucleic acids, in particular to those whose respective mRNA has a low degree of secondary folding, based on the particular total mRNA.

This is a further aspect in order to optimize hybridization between the probes and the mRNA to be detected. This is because mRNA molecules frequently have a secondary structure, which is based on hybridization of individual mRNA regions with intrinsic, other regions. Thus, for example loop or stem-loop structures are formed. Such regions, however, usually hybridize less readily with other nucleic acid molecules, even if the latter are homologous. Regions of this kind can be calculated quite accurately by computer programs directed thereto (see below). Thus, to implement this aspect, the gene whose activity is desired to be determined for an organism of interest should be analyzed by such a program and, in order to obtain a suitable probe—usually comprising only a subsection (see below)—sections should be used for which only a low degree of mRNA secondary structures is predicted.

In preferred embodiments of nucleic acid-binding chips of the invention, one, preferably a plurality, of the probes referred to as being relevant to the invention has/have a length of increasingly preferably less than 200, 150, 125 or 100 nucleotides, preferably from 20 to 60 nucleotides, particularly preferably from 45 to 55 nucleotides.

This is because the probes used for the detection reaction need to comprise only part of the mRNA to be detected, as long as the signal obtainable via them is still specific enough. This specificity, the distinguishability of different mRNAs, sets the lower limit of the length of the probes in question and must, where appropriate, be determined in preliminary experiments.

The identification of suitable probe lengths and probe regions is known per se to the person skilled in the art and is normally carried out with the aid of specialized software. Examples of such software are the programs Array Designer from Premier Biosoft International, USA, and Vector NTI® Suite, V. 7, obtainable from InforMax, Inc., Bethesda, USA. These software programs also take into account, for example, predefined probe lengths and melting temperatures, in addition to the secondary structures already mentioned.

In preferred embodiments of nucleic acid-binding chips of the invention, binding of the mRNA to the concerned probe referred to as being relevant to the invention triggers an electric signal.

The article by J. Wang (Acc. Chem. Res.; ISSN 0001-4842; Rec. Sep. 12, 2001, pp. A-F), mentioned above, discusses the advantages of an electrically analyzable system over an optical system. It also refers to various embodiments of such sensors, which have been developed in the prior art.

Thus the time interval from sampling to measuring the signal is currently approximately 24 h for optically analyzable chips. With the aid of an electrical system, the required time is currently less than 2 h (cf. FIG. 1). In contrast to this, the number of simultaneously analyzable samples is currently in the two-digit range with electrically analyzable chips, but rapid development gives reason to believe that this order of magnitude may be exceeded soon. The limiting factors in this are the electronic evaluation units for the various signals.

An example of a method for quantifying mRNA, which is established in the prior art, is RT-PCT. This method is described in the article “Quantification of Bacterial mRNA by One-Step RT-PCR Using the LightCycler System” (2003) by S. Tobisch, T. Koburger, B. Jürgen, S. Leja, M. Hecker and T. Schweder in BIOCHEMICA, Volume 3, pages 5 to 8. In comparison with this, detection via electrochips has another advantage, namely higher reliability of the data, since these have distinctly smaller ranges of fluctuation compared to RT-PCR.

The preparation of corresponding electronically analyzable chips is described, for example, in the patent applications WO 00/62048 A2, WO 00/67026 A1 and WO 02/41992, whose entire disclosed content is incorporated into the present application.

The manner in which function electrically readable chips of a particularly preferred embodiment can be described as follows: the gene-specific probes are bound covalently in a manner known per se to magnetic beads located in specifically designed chambers of said chips. The specific hybridization of the corresponding mRNA on the particular beads occurs in this hybridization chamber whose temperature can be controlled and which can be flushed by the solutions in question. The beads are retained in this chamber by a magnet. After hybridization of the RNA samples on the beads-bound to the DNA probes, unbound RNA is removed in a washing step, as a result of which only specific hybrids are still present in the incubation chamber, in fact bound to the magnetic beads.

After washing, a detection probe labeled by a biotin-extravidin-bound alkaline phosphatase is introduced into the incubation chamber. This probe binds to a second free region of the hybridized mRNA. This hybrid is then washed again and incubated with the alkaline phosphatase substrate, para-aminophenol phosphate (pAPP). The enzymatic reaction in the incubation chamber releases the redox-active product, para-aminophenol (pAP), which is then passed over the Red/Ox electrode on the electrical chip and the signal is transmitted to a potentiostat.

A system-specific software (for example MCDDE32) reads the obtained data, and the results may be evaluated and displayed on a computer with the aid of a further program (for example Origin).

This process can of course be varied with regard to both the technical design of the chips and the evaluation. Thus, for example, the detection reaction may be carried out by way of a different reaction, but preferably a redox reaction due to the electrical principle of measurement.

One achievement of the present invention is to have identified nitrogen metabolism-specific and thus process-critical genes and to have made them accessible to analysis via correspondingly designed biochips. The advantage of chips over conventional detection methods comprises, in addition to the time saved and higher accuracy, the possibility of detecting the activities of a plurality of different genes in the same sample at the same time by providing a plurality of probes on a single support and of said activities resulting in a more solid and more detailed picture, for example with regard to the time at which a nitrogen deficiency started, with the application to a special problem, described herein.

A separate subject matter of the invention is therefore the simultaneous use of nucleic acid probes or nucleic acid analog probes for at least three of the following 50 genes:

kdgR, citA, gene coding for a putative protein (putative ABC transporter/amino acid permease) (homolog to SEQ ID NO. 91), htrA, ycnl, gene coding for a hypothetical protein of unknown function (homolog to SEQ ID NO. 41), gene coding for a hypothetical protein of unknown function (homolog to SEQ ID NO. 53), yppF, yqjN, ggt, alsR, glnA, nrgA, yciC, yvtA, nrgB, gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA, bound to a nucleic acid-binding chip, preferably to the same chip, for determining the physiological state of an organism undergoing a biological process.

As explained above, these 50 genes are selected so as to deliver a picture of the situation of the nitrogen metabolism of the observed organism, since they are, as described in Examples 1 to 3 and 5, significantly and comparatively specifically induced during transition of the Gram-positive bacterium B. licheniformis to a nitrogen deficiency condition. A comparable statement can also be expected for other organisms, which have the homologous genes or proteins with essentially the same metabolically relevant properties.

As likewise described in detail above, such gene activities may in principle be determined in various ways, for example by Northern hybridization. However, analysis with the aid of a nucleic acid-binding chip, in particular an above-described chip, enables a plurality of gene activities to be determined very efficiently at the same time and, moreover, very much in real-time. As a result, the metabolic alterations of an organism undergoing a biological process may be observed in real time and, where appropriate, intervened in a regulatory manner.

The above statements on nucleic acid-binding chips apply to the uses accordingly specified herein of the probes in question.

Accordingly, preference is given here to those uses, wherein the total number of all the different nitrogen metabolism-specific probes does not exceed 80.

Accordingly, further preference is given here to those uses, wherein the probes indicated above as inventive are employed with increasing preference in the order indicated above as preferable (inverse to the order in Table 3 including example 5).

In accordance with the above statements, increasing preference is given to the following of the above-specified uses of nucleic acid probes or nucleic acid analog probes:

use, wherein at least one, increasingly preferably two or three of the nucleic acid- or nucleic acid analog-probes is/are selected from the following 34 genes: gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA,

among these, preferably for genes from the following 20 genes:

trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), tnrA,

among these, particularly preferably for genes from the following 12 genes:

nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81),

among these, quite particularly preferably for genes from the following 4 genes:

gene coding for a putative protein (putative nitrogen regulation protein P-II) (homolog to SEQ ID NO. 93), nasF, yrkC, gene coding for a hypothetical protein of unknown function (homolog to SEQ ID NO. 81).

In accordance with the above statements, the inventive uses preferably concern the simultaneous use of at least 4, 5, 6, 7, 8, 9, 10, 12, 14, 16, 18, 20, 22, 24, 26 28, 30, 35, 40, 45 or 50 of the specified probes.

In accordance with the above statements, preferred uses of the invention are those, wherein the total number of all the different probes does, with increasing preference, not exceed 80, 75, 70, 65, 60, 55, 50, 40, 30, 20 or 10.

In accordance with the above statements, further preference is given to uses of the invention for determining a change in the nitrogen metabolism of the organism undergoing the biological process, preferably for detecting a nitrogen deficiency condition.

In accordance with the above statements, further preference is given to uses of the invention, wherein at least one, increasingly preferably a plurality, of the specified probes is/are derived from the sequences listed in the sequence listing under the numbers SEQ ID No. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97 and 99.

A separate subject matter of the invention is formed by methods for the determination of the physiological state of an organism under a biological process by the use of a nucleic acid-binding chip of the invention.

These methods in principle comprise removing samples of the observed organism during said process, without interrupting the latter, and isolating the mRNA from said samples. These compounds or, where appropriate, compounds derived therefrom, such as cDNA, for example, are passed across an above-described nucleic acid-binding chip which is treated taking into account the method steps indicated above—such as, for example, adequate incubation time or washing off non-specifically binding nucleic acids—and is finally delivered to the detection apparatus. A method protocol of this kind is depicted in principle in FIG. 1 on the basis of the example of electrically analyzable chips.

The above comments on nucleic acid-binding chips apply accordingly to the methods specified herein for the determination of the physiological state of an organism undergoing a biological process.

Preference is given to methods of the invention, wherein a change in the nitrogen metabolism of the organism undergoing the biological process, preferably a nitrogen deficiency condition, is determined.

With respect to this field of use, the genes described in the examples and listed above have been selected. Their significant induction, at least in B. licheniformis, is accompanied by the onset of a nitrogen deficiency condition, such that this can be detected particularly reliably by the cited method, and in fact not only in B. Licheniformis but, with increasingly improving prospects of success, also with increasingly related species (see above). Moreover, this metabolic situation represents a critical point in the lifecycle of many microorganisms. Thus, as illustrated in the examples (see below), the particular onset of nitrogen deficiency was always also associated with a transition to the stationary growth phase. Methods, which facilitate the early recognition of this point in time, serve to delay this transition and, in particular with industrially utilized fermentations, prolong the phase of production of a valuable substance, particularly when dealing with a nitrogen-containing valuable product such as for example a protein.

A further possibility for conducting the method for the cultivation of microorganisms consists of supplying, beside the first (and therefore better) utilized N-source, a further source. In the phase of the initially observed nitrogen deficiency, the concerned cells, in so far as they are capable, turn themselves to using the second N source that is less utilizable for them. With this transition there is generally observed a decrease in the growth rate, which can be detected at an early stage by means of the inventive method.

In accordance with the above, preference is given to those inventive methods, wherein the organism selected for the bioprocess is a representative of unicellular eukaryotes, Gram-positive or Gram-negative bacteria.

In accordance with the above statements, preference is given among said methods to those methods of the invention, wherein the unicellular eukaryotes are protozoa or fungi, among these in particular yeast, quite particularly Saccharomyces or Schizosaccharomyces.

In accordance with the above statements, preference is given here also to those inventive methods, wherein the Gram-positive bacteria are coryneform bacteria or those of the genera Staphylococcus, Corynebacteria and Bacillus, in particular of the species Staphylococcus carnosus, Corynebacterium glutamicum, Bacillus subtilis, B. licheniformis, B. amyloliquefaciens, B. agaradherens, B. stearothermophilus, B. globigii or B. lentus, and quite particularly B. licheniformis.

In accordance with the above statements, no less preference is given here also to those inventive methods, wherein the Gram-negative bacteria are those of the genera E. coli and Klebsiella, in particular derivatives of Escherichia coli K122, of Escherichia coli B or Klebsiella planticola, and quite particularly derivatives of the strains Escherichia coli BL21 (DE3), E. coli RV308, E. coli DH5α, E. coli JM109, E. coli XL-1 or Klebsiella planticola αRf).

In accordance with the above statements, preference is given to those methods of the invention, wherein those specified probes are used, which are derived from the SEQ ID Nos. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97 or 99 indicated in the sequence listing.

Among these, preference is again given to those methods, which employ the probes specified herein for the above-listed Gram-positive bacteria, in particular B. licheniformis, since these sequences have been isolated from this very organism and can therefore be applied most successfully to this species.

In accordance with the above statements, preference is given to those inventive methods, wherein the physiological state is determined at various times of the same process, preferably using a plurality of structurally identical nucleic acid-binding chips, particularly preferably of the same nucleic acid-binding chip.

In accordance with the above statements, preference is furthermore given to those methods of the invention, wherein the process is a fermentation, in particular the fermentative production of a commercially usable product, particularly preferably the production of a protein or of a low molecular weight chemical compound.

In accordance with the above statements, preference is given among these methods to those methods of the invention, wherein the low molecular weight chemical compound is a natural substance, a food supplement or a pharmaceutically relevant compound.

In accordance with the above statements, preference is alternatively also given to those methods of the invention, wherein the protein is an enzyme, in particular an enzyme from the group of the α-amylases, proteases, cellulases, lipases, oxidoreductases, peroxidases, laccases, oxidases and hemicellulases.

A separate subject matter of the invention is also the possible uses of inventive nucleic acid-binding chips, as have been described in detail above, for determining the physiological state of an organism undergoing a biological process.

The above comments on nucleic acid-binding chips apply accordingly to the uses specified herein for determining the physiological state of an organism undergoing a biological process.

In accordance with the above statements, preference is given to methods of the invention, wherein a change in the nitrogen metabolism of the organism undergoing the biological process, preferably a nitrogen deficiency condition, is determined.

In accordance with the above statements, further preference is given to those inventive methods, wherein the organism selected for the bioprocess is a representative of unicellular eukaryotes, Gram-positive or Gram-negative bacteria.

In accordance with the above statements, such inventive methods among these are preferred, wherein the unicellular eukaryotes are protozoa or fungi, among these in particular yeast, quite particularly Saccharomyces or Schizosaccharomyces.

In accordance with the above statements, preference is alternatively given here to those inventive methods, wherein the Gram-positive bacteria are coryneform bacteria or those of the genera Staphylococcus, Corynebacteria and Bacillus, in particular of the species Staphylococcus carnosus, Corynebacterium glutamicum, Bacillus subtilis, B. licheniformis, B. amyloliquefaciens, B. agaradherens, B. stearothermophilus, B. globigii or B. lentus, and quite particularly B. licheniformis.

In accordance with the above statements, no less preference is given here also to those inventive methods, wherein the Gram-negative bacteria are those of the genera E. coli and Klebsiella, in particular derivatives of Escherichia coli K122, of Escherichia coli B or Klebsiella planticola, and quite particularly derivatives of the strains Escherichia coli BL21 (DE3), E. coli RV308, E. coli DH5α, E. coli JM109, E. coli XL-1 or Klebsiella planticola (Rf).

In accordance with the above statements, preference is given to those methods of the invention, wherein those specified probes are used, which are derived from the SEQ ID Nos. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97 or 99 indicated in the sequence listing.

For the reason already specified above, among these uses, preference is given in turn to those uses which employ the probes specified herein for the above-listed Gram-positive bacteria, in particular B. licheniformis.

In accordance with the above statements, preference is given to those inventive methods, wherein the physiological state is determined at various times of the same process, preferably using a plurality of structurally identical nucleic acid-binding chips, particularly preferably of the same nucleic acid-binding chip.

In accordance with the above statements, preference is furthermore given to those methods of the invention, wherein the process is a fermentation, in particular the fermentative production of a commercially usable product, particularly preferably the production of a protein or of a low molecular weight chemical compound.

In accordance with the above statements, preference is given among these methods to those methods of the invention, wherein the low molecular weight chemical compound is a natural substance, a food supplement or a pharmaceutically relevant compound.

In accordance with the above statements, preference is alternatively also given to those methods of the invention, wherein the protein is an enzyme, in particular an enzyme from the group of the α-amylases, proteases, cellulases, lipases, oxidoreductases, peroxidases, laccases, oxidases and hemicellulases.

The present invention is additionally illustrated by the following examples.

### EXAMPLES

All molecular-biological work was carried out by standard methods as can be found, for example, in the manual by Fritsch, Sambrook and Maniatis “Molecular cloning: a laboratory manual”, Cold Spring Harbour Laboratory Press, New York, 1989, or comparable specialist literature. Enzymes and kits were used according to the instructions of the particular manufacturer.

### Example 1

### Identification of the Gene Probes by Chip Analyses

### Culturing of Bacteria and Isolation of Samples

Cells of the Bacillus licheniformis DSM13 strain (obtainable from Deutsche Sammlung von Mikroorganismen and Zellkulturen GmbH, Mascheroder Weg 1b, 38124 Braunschweig, Germany; http://www.dsmz.de) were cultured in nitrogen-limited, synthetic Belitsky minimal medium (1 mM final concentration) with constant shaking at 270 rpm and 37° C. This medium has the following composition: (1.) Basic medium (pH 7.5): 0.015 M (NH₄)₂SO₄, 0.008 M MgSO₄×7H₂O, 0.027 M KCl, 0.007 M Na₃citrate×2H₂O, 0.050 M Tris-HCl and 0.009 M glutamic acid; (2.) supplements: 0.2 M KH₂PO₄, 0.039 M L-tryptophan-HCl, 1 M CaCl₂×2H₂O, 0.0005 M FeSO₄×7H₂O, 0.025 M MnSO₄×4H₂O and 20% (w/v) glucose; and as (3.) medium supplementing plan: 0.14 ml KH₂PO₄, 0.2 ml CaCl₂, 0.2 ml FeSO₄, 0.04 ml MnSO₄, 1 ml glucose. All quantities refer to 100 ml of basic medium.

In the course of establishing the growth profile, the control sample was removed at an optical density at 500 nm (OD₅₀₀) of from 0.4 to 0.5, with another sample (“transition”) being removed in the transient growth phase at OD₅₀₀ 0.8 to 0.9. The supplementing plan for the Belitzky minimal medium, indicated above, was set up in such a way that nitrogen deficiency started at OD₅₀₀ 0.8 to 1.0, thereby introducing the stationary phase. Further samples were taken after another 30, 60, 90 and 120 min. Immediately after their removal, an equal volume of Killing buffer (20 mM NaN₃; 20 mM Tris-HCl, pH 7.5; 5 mM MgCl₂) cooled to 0° C. was added to the samples. Immediately thereafter, the cell pellet was removed by centrifugation at 4° C. and 15000 rpm for 5 min, the supernatant was discarded, and the pellet was either frozen at −80° C. or immediately worked up further.

### Cell Disruption

The cells were disrupted using the “Hybaid RiboLyser™ Cell Disruptor” (Thermo Electron Corporation, Dreieich, Germany). This method is based on mechanically destroying the cell wall and the cell membrane with the aid of glass beads of approx. 0.1 mm in size (Sartorius BBI, Melsungen, Germany). The cells to be disrupted which had been resuspended in lysis buffer II (3 mM EDTA; 200 mM NaCl) beforehand were introduced together with the glass beads into a glass (strain maintenance) tube. Acidic phenol was also added in order to prevent RNA degradation by RNases. This tube was then clamped inside the RiboLyser, wherein the glass beads collided with the cells under vigorous shaking, thereby causing disruption of the cells.

### Isolation of Total RNA

After cell disruption, the RNA-containing aqueous phase is separated by centrifugation from the protein and cell fragments, chromosomal DNA and the glass beads, and the RNA is isolated therefrom with the aid of the KingFisher mL apparatus (Thermo Electron Corporation, Dreieich, Germany) using the MagNA Pure LC RNA Isolation Kit I (Roche Diagnostics, Penzberg, Germany). This purification is based on the RNA binding to magnetic glass particles in the presence of chaotropic salts that also inactivate RNases. The magnetic particles here serve as a means of transporting the RNA between various reaction vessels filled with binding, washing and elution buffers. The KingFisher mL utilized for this is a kind of pipetting robot that transports the particles with the bound RNA back and forth between the vessels with the aid of magnets and is also utilized for mixing the samples. Finally, the RNA is detached from the magnetic particles and is then present in a purified state.

### RNA Quality and Quantity Control

The purification of RNA is followed by quality and quantity controls. The Agilent Bioanalyzer 2100 apparatus (Agilent Technologies, Berlin, Germany) utilized for this enables the analysis of RNA on lab-on-a-chip scale. Together with the Agilent “RNA 6000 Nano Kit”, total RNA is fractionated gel electrophoretically and thereby offers the possibility of examining the quality with regard to partial degradation and contaminations. This involves detecting ribosomal RNA (16 S and 23 S rRNA). If the latter appear as clear bands, the RNA can be assumed not to have been degraded during processing and therefore to be intact and to be able to be introduced into the subsequent examinations. In addition, the exact concentration was also determined here.

### Transcriptome Analyses

Transcriptome analyses were carried out using total genomic B. licheniformis DSM13 DNA microarrays which had been prepared in the usual manner (for example according to WO 95/11995 A1) and which can be analyzed by an optical system. These DNA microarrays contained two copies of virtually each B. licheniformis gene, such that it was possible to analyze two samples in parallel on the same chip and to average the values obtained.

The principle of the measurement carried out consists of transcribing in vitro the particular mRNA molecules from the sample taken via reverse transcription to DNA, wherein one of the added deoxyribonucleotides carries a dye marker. These labeled molecules are then hybridized with the known probes located at known sites of the chip, and the particular strength of the signal at the sites in question, which can be attributed to the fluorescent marker, is optically recorded. When using two different fluorescent markers for a control and for a sample actually to be studied, which are made to hybridize simultaneously and thereby compete for binding to the presented probe, different color values are obtained which provide a measure of the ratio of the concentration of the control to that of the sample.

dUTP labeled with the fluorescent dye cyanine 3 or cyanine 5 was chosen for this labeling. Thus, in each case 25 pg of total RNA of the control (OD₅₀₀ 0.4) were labeled with the fluorescent dye cyanine 3 (Amersham Biosciences Europe GmbH, Freiburg, Germany) and 25 μg of total RNA of the particular stress sample (transient phase, 30, 60 and 120 min) were labeled with the fluorescent dye cyanine 5 (GE Healthcare, Freiburg, Germany). Competitive hybridization of the two samples was carried out at 42° C. on the conventional, total genomic B. licheniformis DSM13 DNA microarray for at least 16 hours.

After washing the array in order to remove unspecific bonds, the array was read out optically with the aid of the ScanArray® Express Laser Scanner (PerkinElmer Life and Analytical Sciences, Rodgau-Jügesheim, Germany). All hybridizations were repeated, with the samples being labeled with the in each case other dye (dye swap method). Quantitative evaluation of the arrays was carried out using the ScanArray® Express software (available from PerkinElmer Life and Analytical Sciences, Rodgau-Jügesheim, Germany) according to the manufacturer's instructions and with standard parameters.

The arrays were normalized and evaluated with the aid of the Lucidea Score Card controls (GE Healthcare, Freiburg, Germany). With the aid of this “Score Card” which is used for controlling hybridization efficiency and quality, known control DNAs and “spikes” in the form of oligos have been applied to the array according to the manufacturer's instructions and admixed with complementary sequences during hybridization. Thus it was possible to control, after scanning, the success of said hybridization and incorporation of the dyes. The controls should be present in the same amounts in both samples and therefore appear yellow after scanning or have a ratio of 1 between both channels. The spikes are specific for the particular sample and are applied in various dilutions, i.e. they appear red or green after scanning for the particular sample.

For expression of the genes, averages of the two hybridizations and the particular standard deviations were calculated. For a significant induction or significant repression, the following thresholds of the ratio of the amount of RNA of the particular gene to the control value were observed: genes whose RNA has a ratio of >3 (i.e. at least a three-fold increase) are regarded as induced; genes having an RNA ratio of <0.3 (i.e. a decrease to less than 30%) are distinctly repressed. These results are listed in Example 2.

### Example 2

### Genes Induced Under Nitrogen Deficiency

Table 1 below lists all 210 Bacillus licheniformis DSM13 genes determined in Example 1 whose induction (of at least a factor of 3) was observed under the conditions of nitrogen deficiency described in Example 1. The first two columns indicate the particular name of the derived protein and, respectively, its abbreviation (if available); the “BLi number” corresponds to the “locus_tag” of the B. licheniformis complete genome accessible under the entry AE017333 (bases 1 to 4 222 645) in the GenBank database (National Center for Biotechnology Information NCBI, National Institutes of Health, Bethesda, Md., USA; http://www.ncbi.nlm.nih.gov; as of 12.2.2004); this is followed by the factors of increasing the concentration of the in each case corresponding mRNAs, observed at the times indicated at the top.

TABLE 1
The 210 Bacillus licheniformis DSM13 genes determined in Example
1, whose induction (of at least a factor of 3) under nitrogen
deficiency was observed (explanations: see text).

| Protein name/Gene | | | | | | | |
|---|---|---|---|---|---|---|---|
| function | Gene | BLi—Nr | Transition | 30 min | 60 min | 90 min | 120 min |
| unknown function - | yqjN | BLi02550 | 1.56 | 2.68 | 4.48 | 8.50 | 8.65 |
| similar to amino acid | | | | | | | |
| degradation | | | | | | | |
| unknown function | gene for a | BLi02843 | 1.03 | 1.74 | 2.72 | 3.36 | 3.32 |
| | hypothetical | | | | | | |
| | protein | | | | | | |
| unknown function - | yozO | BLi02247 | 1.14 | 2.67 | 2.38 | 3.17 | 3.05 |
| similar to unknown | | | | | | | |
| function proteins | | | | | | | |
| aldehyde | dhaS | BLi02249 | 1.60 | 4.31 | 6.38 | 5.59 | 5.99 |
| dehydrogenase | | | | | | | |
| bacillopeptidase F | bpr | BLi01748 | 1.43 | 6.26 | 4.37 | 7.60 | 12.43 |
| putative | gene for a | BLi01747 | 1.17 | 3.58 | 2.49 | 3.41 | 3.63 |
| bacillopeptidase F | putative | | | | | | |
| | protein | | | | | | |
| unknown function - | yvyD | BLi03774 | 1.60 | 10.78 | 9.72 | 11.51 | 8.37 |
| similar to sigma-54 | | | | | | | |
| modulating factor of | | | | | | | |
| gram-negative bacteria | | | | | | | |
| unknown function | gene for a | BLi00431 | 1.23 | 4.74 | 4.29 | 4.97 | 3.47 |
| | hypothetical | | | | | | |
| | protein | | | | | | |
| nitrogen-regulated PII- | nrgB | BLi03889 | 3.61 | 6.92 | 5.04 | 9.99 | 6.67 |
| like protein | | | | | | | |
| ammonium transporter | nrgA | BLi03888 | 3.04 | 5.71 | 3.91 | 9.56 | 7.31 |
| unknown function | gene for a | BLi00491 | 2.56 | 3.79 | 3.15 | 3.71 | 2.75 |
| | conserved | | | | | | |
| | hypothetical | | | | | | |
| | protein | | | | | | |
| unknown function | yoeB | BLi01308 | 1.31 | 22.73 | 25.86 | 46.66 | 39.40 |
| unknown function - | yjcG | BLi01284 | 1.23 | 3.28 | 2.71 | 3.07 | 2.42 |
| similar to unknown | | | | | | | |
| function proteins | | | | | | | |
| unknown function - | ykfA | BLi01397 | 1.86 | 3.48 | 4.44 | 3.15 | 3.61 |
| similar to immunity to | | | | | | | |
| bacteriotoxins | | | | | | | |
| dipeptide ABC | dppE | BLi01396 | 2.60 | 8.07 | 8.32 | 5.59 | 6.85 |
| transporter/dipeptide- | | | | | | | |
| binding protein | | | | | | | |
| (sporulation) | | | | | | | |
| dipeptide ABC | dppC | BLi01394 | 2.08 | 4.08 | 4.62 | 3.16 | 3.40 |
| transporter/permease | | | | | | | |
| (sporulation) | | | | | | | |
| dipeptide ABC | dppB | BLi01393 | 2.47 | 4.19 | 3.35 | 2.13 | 2.46 |
| transporter/permease | | | | | | | |
| (sporulation) | | | | | | | |
| D-alanyl- | dppA | BLi01392 | 2.39 | 3.78 | 3.51 | 2.30 | 2.64 |
| aminopeptidase | | | | | | | |
| pyrroline-5-carboxylate | proG | BLi01391 | 1.72 | 4.49 | 3.23 | 1.54 | 1.72 |
| reductase | | | | | | | |
| serine protease Do | htrA | BLi01390 | 7.91 | 8.10 | 2.03 | 1.93 | 1.67 |
| (heat-shock protein) | | | | | | | |
| unknown function | gene for a | BLi01389 | 4.31 | 4.00 | 1.87 | 1.93 | 2.03 |
| | hypothetical | | | | | | |
| | protein | | | | | | |
| unknown function - | ypjP | BLi02321 | 1.33 | 3.12 | 4.80 | 3.44 | 3.93 |
| similar to unknown | | | | | | | |
| function proteins | | | | | | | |
| unknown function - | yplQ | BLi02317 | 1.10 | 2.44 | 2.85 | 3.31 | 3.10 |
| similar to hemolysin III | | | | | | | |
| homolog | | | | | | | |
| unknown function - | yppQ | BLi02302 | 1.58 | 4.72 | 4.17 | 5.38 | 3.63 |
| similar to peptide | | | | | | | |
| methionine sulfoxide | | | | | | | |
| reductase | | | | | | | |
| unknown function - | yusX | BLi03477 | 1.31 | 2.92 | 3.22 | 3.21 | 2.95 |
| similar to | | | | | | | |
| oligoendopeptidase | | | | | | | |
| major intracellular | ispA | BLi01423 | 1.11 | 6.98 | 4.30 | 17.19 | 4.56 |
| serine protease | | | | | | | |
| negative regulator of | rsiX | BLi02448 | 2.06 | 4.92 | 2.62 | 5.37 | 5.95 |
| sigma-X activity | | | | | | | |
| RNA polymerase ECF- | sigX | BLi02449 | 2.24 | 5.73 | 2.18 | 5.40 | 4.34 |
| type sigma factor | | | | | | | |
| anti-sigma F factor (EC | spoIIAB | BLi02496 | 2.37 | 7.05 | 4.42 | 4.37 | 2.02 |
| 2.7.1.37)/stage II | | | | | | | |
| sporulation protein AB | | | | | | | |
| anti-sigma F factor | spoIIAA | BLi02497 | 1.43 | 4.60 | 3.37 | 3.17 | 2.24 |
| antagonist/stage II | | | | | | | |
| sporulation protein AA | | | | | | | |
| unknown function - | ysdB | BLi03031 | 1.42 | 2.88 | 2.55 | 4.02 | 4.69 |
| similar to unknown | | | | | | | |
| function proteins | | | | | | | |
| subtilisin carlsberg | aprE | BLi01109 | 1.74 | 13.83 | 5.92 | 18.92 | 7.74 |
| precursor (EC | | | | | | | |
| 3.4.21.62) | | | | | | | |
| unknown function | gene for a | BLi00303 | 1.20 | 4.92 | 2.55 | 5.06 | 4.40 |
| | hypothetical | | | | | | |
| | protein | | | | | | |
| unknown function | ybdN | BLi00302 | 1.71 | 4.07 | 2.95 | 7.30 | 5.02 |
| putative serine protease | gene for a | BLi00301 | 2.31 | 13.40 | 7.51 | 15.91 | 11.55 |
| | putative | | | | | | |
| | protein | | | | | | |
| unknown function - | yoaD | BLi04062 | 2.39 | 1.82 | 2.44 | 4.37 | 4.19 |
| similar to | | | | | | | |
| phosphoglycerate | | | | | | | |
| dehydrogenase | | | | | | | |
| putative transcriptional | gene for a | BLi04093 | 1.25 | 2.63 | 2.96 | 3.01 | 2.80 |
| regulator | putative | | | | | | |
| | protein | | | | | | |
| citrate synthase III | mmgD | BLi04094 | 1.32 | 5.34 | 4.06 | 6.95 | 3.45 |
| function unknown | mmgE | BLi04095 | 0.95 | 2.34 | 3.19 | 3.60 | 3.10 |
| function | | | | | | | |
| unknown function - | yqiQ | BLi04096 | 1.11 | 2.03 | 3.13 | 2.77 | 3.21 |
| similar to | | | | | | | |
| phosphoenolpyruvate | | | | | | | |
| mutase | | | | | | | |
| RNA polymerase ECF- | sigW | BLi00199 | 1.04 | 1.61 | 3.11 | 3.58 | 4.83 |
| type sigma factor | | | | | | | |
| unknown function - | ybbM | BLi00200 | 1.01 | 1.79 | 2.91 | 3.84 | 4.17 |
| similar to unknown | | | | | | | |
| function proteins | | | | | | | |
| unknown function | gene for a | BLi02331 | 0.98 | 1.45 | 1.93 | 4.09 | 3.26 |
| | hypothetical | | | | | | |
| | protein | | | | | | |
| unknown function | gene for a | BLi01570 | 1.33 | 3.57 | 1.86 | 3.01 | 1.74 |
| | hypothetical | | | | | | |
| | protein | | | | | | |
| phage shock protein A | pspA | BLi00640 | 1.59 | 2.26 | 3.17 | 2.88 | 3.78 |
| homolog | | | | | | | |
| unknown function | yfmQ | BLi00629 | 1.38 | 3.99 | 2.78 | 5.91 | 1.88 |
| transcriptional repressor | glnR | BLi01992 | 4.38 | 11.07 | 7.61 | 9.77 | 8.25 |
| of the glutamine | | | | | | | |
| synthetase gene | | | | | | | |
| glutamine synthetase | glnA | BLi01993 | 5.69 | 9.51 | 8.60 | 9.06 | 8.61 |
| unknown function | yoaW | BLi02014 | 1.16 | 2.80 | 3.04 | 3.22 | 4.52 |
| unknown function - | ydfO | BLi02024 | 1.86 | 3.64 | 3.36 | 3.89 | 3.07 |
| similar to unknown | | | | | | | |
| function proteins | | | | | | | |
| unknown function - | ywfL | BLi03988 | 2.21 | 2.33 | 2.84 | 5.85 | 4.75 |
| similar to unknown | | | | | | | |
| function proteins | | | | | | | |
| putative | gene for a | BLi03989 | 2.19 | 2.76 | 3.89 | 6.36 | 7.16 |
| hydroxybenzoate | putative | | | | | | |
| hydroxylase | protein | | | | | | |
| putative benzoate | gene for a | BLi03990 | 4.12 | 4.74 | 6.49 | 13.68 | 12.33 |
| transport protein | putative | | | | | | |
| | protein | | | | | | |
| putative aromatic | gene for a | BLi03991 | 3.29 | 4.33 | 3.86 | 13.22 | 12.18 |
| compounds specific | putative | | | | | | |
| dioxygenase | protein | | | | | | |
| putative decarboxylase/ | gene for a | BLi03993 | 3.84 | 5.22 | 10.49 | 22.93 | 24.21 |
| dehydratase | putative | | | | | | |
| | protein | | | | | | |
| close homolog to DhaS | gene for a | BLi03994 | 5.33 | 9.09 | 10.32 | 22.37 | 19.12 |
| aldehyde | hypothetical | | | | | | |
| dehydrogenase | protein | | | | | | |
| putative transcriptional | gene for a | BLi03995 | 1.82 | 5.01 | 7.61 | 12.47 | 15.76 |
| regulator | putative | | | | | | |
| | protein | | | | | | |
| unknown function | gene for a | BLi03996 | 1.52 | 2.34 | 4.24 | 8.76 | 10.13 |
| | conserved | | | | | | |
| | hypothetical | | | | | | |
| | protein | | | | | | |
| putative phage protein | gene for a | BLi01482 | 1.07 | 2.69 | 2.55 | 3.27 | 3.19 |
| | putative | | | | | | |
| | protein | | | | | | |
| putative phage protein | gene for a | BLi01480 | 1.26 | 2.64 | 2.69 | 3.60 | 3.37 |
| | putative | | | | | | |
| | protein | | | | | | |
| putative phage capsid | gene for a | BLi01470 | 3.21 | 14.00 | 9.43 | 9.07 | 5.59 |
| protein | putative | | | | | | |
| | protein | | | | | | |
| putative phage protein | gene for a | BLi01466 | 2.04 | 2.57 | 3.21 | 6.12 | 5.04 |
| | putative | | | | | | |
| | protein | | | | | | |
| putative portal protein | gene for a | BLi01465 | 1.85 | 3.40 | 3.52 | 6.43 | 5.22 |
| | putative | | | | | | |
| | protein | | | | | | |
| unknown function | ykoP | BLi03288 | 1.66 | 3.60 | 2.04 | 3.67 | 1.90 |
| metalloregulation DNA- | mrgA | BLi03480 | 3.03 | 3.23 | 2.60 | 1.62 | 1.57 |
| binding stress protein | | | | | | | |
| unknown function - | yvtA | BLi03481 | 9.99 | 3.41 | 1.30 | 1.61 | 1.00 |
| similar to HtrA-like | | | | | | | |
| serine protease | | | | | | | |
| unknown function - | yvqH | BLi03496 | 0.96 | 3.55 | 9.12 | 12.94 | 22.11 |
| similar to unknown | | | | | | | |
| function proteins from | | | | | | | |
| B. subtilis | | | | | | | |
| unknown function | yvqI | BLi03497 | 0.96 | 2.79 | 6.83 | 10.39 | 21.62 |
| unknown function - | ycbA | BLi00275 | 0.90 | 1.43 | 2.31 | 3.81 | 5.46 |
| similar to two- | | | | | | | |
| component sensor | | | | | | | |
| histidine kinase [YcbB] | | | | | | | |
| transcriptional repressor | kdgR | BLi03828 | 0.77 | 1.99 | 2.15 | 4.81 | 8.02 |
| of the pectin utilization | | | | | | | |
| operon | | | | | | | |
| unknown function - | ydhQ | BLi02559 | 1.02 | 1.31 | 1.56 | 3.12 | 3.48 |
| similar to transcriptional | | | | | | | |
| regulator (GntR family) | | | | | | | |
| unknown function - | ycnI | BLi00479 | 3.74 | 5.12 | 3.84 | 8.23 | 3.18 |
| similar to unknown | | | | | | | |
| function proteins | | | | | | | |
| unknown function - | ycnJ | BLi00480 | 4.04 | 5.28 | 4.59 | 10.74 | 3.79 |
| similar to copper export | | | | | | | |
| protein | | | | | | | |
| unknown function - | ycnK | BLi00481 | 4.60 | 10.14 | 8.17 | 14.46 | 8.30 |
| similar to transcriptional | | | | | | | |
| regulator (DeoR family) | | | | | | | |
| assimilatory nitrate | nasB | BLi00482 | 18.17 | 22.63 | 20.62 | 25.24 | 16.47 |
| reductase (electron | | | | | | | |
| transfer subunit) | | | | | | | |
| assimilatory nitrate | nasC | BLi00483 | 11.07 | 22.08 | 20.33 | 21.01 | 20.53 |
| reductase (catalytic | | | | | | | |
| subunit) | | | | | | | |
| assimilatory nitrite | nasD | BLi00484 | 6.63 | 10.58 | 15.49 | 20.64 | 12.06 |
| reductase (subunit) | | | | | | | |
| assimilatory nitrite | nasE | BLi00485 | 10.63 | 23.53 | 27.51 | 42.63 | 21.86 |
| reductase (subunit) | | | | | | | |
| uroporphyrin-III C- | nasF | BLi00486 | 8.08 | 37.38 | 22.44 | 36.50 | 18.60 |
| methyltransferase | | | | | | | |
| unknown function | gene for a | BLi00236 | 2.98 | 5.18 | 6.04 | 7.59 | 8.29 |
| | hypothetical | | | | | | |
| | protein | | | | | | |
| unknown function - | yckI | BLi00418 | 3.21 | 3.55 | 3.01 | 3.09 | 2.03 |
| similar to glutamine | | | | | | | |
| ABC transporter (ATP- | | | | | | | |
| binding protein) | | | | | | | |
| unknown function | gene for a | BLi00413 | 4.35 | 4.40 | 3.28 | 5.49 | 3.05 |
| | hypothetical | | | | | | |
| | protein | | | | | | |
| phosphoenolpyruvate | pckA | BLi03197 | 0.90 | 5.90 | 7.35 | 28.58 | 10.77 |
| carboxykinase | | | | | | | |
| translocation-dependent | tasA | BLi02637 | 4.15 | 3.31 | 1.71 | 4.69 | 1.35 |
| antimicrobial spore | | | | | | | |
| component | | | | | | | |
| unknown function | yqxM | BLi02639 | 2.63 | 3.46 | 1.74 | 4.87 | 1.44 |
| nuclease inhibitor | dinB | BLi02244 | 1.44 | 5.18 | 6.35 | 5.81 | 5.65 |
| unknown function - | yngK | BLi02129 | 1.33 | 4.09 | 4.09 | 5.89 | 4.03 |
| similar to unknown | | | | | | | |
| function proteins | | | | | | | |
| unknown function | gene for a | BLi04184 | 1.97 | 5.16 | 5.07 | 4.72 | 4.39 |
| | hypothetical | | | | | | |
| | protein | | | | | | |
| unknown function | gene for a | BLi04185 | 1.24 | 2.90 | 2.25 | 6.77 | 5.26 |
| | hypothetical | | | | | | |
| | protein | | | | | | |
| putative malate | gene for a | BLi04208 | 1.00 | 5.95 | 3.71 | 16.98 | 2.98 |
| synthase (EC 4.1.3.2) | putative | | | | | | |
| | protein | | | | | | |
| unknown function - | yvcK | BLi03724 | 1.54 | 2.78 | 3.11 | 2.83 | 3.06 |
| similar to unknown | | | | | | | |
| function proteins | | | | | | | |
| ATP-dependent Clp | clpP | BLi03710 | 2.62 | 4.39 | 3.50 | 2.56 | 1.85 |
| protease proteolytic | | | | | | | |
| subunit (class III heat- | | | | | | | |
| shock protein) | | | | | | | |
| unknown function | ykyB | BLi01615 | 1.09 | 2.14 | 2.89 | 4.52 | 3.35 |
| unknown function | gene for a | BLi02232 | 1.07 | 1.66 | 4.21 | 4.17 | 3.71 |
| | hypothetical | | | | | | |
| | protein | | | | | | |
| putative hydrolase | gene for a | BLi02233 | 1.29 | 3.38 | 8.50 | 12.34 | 6.57 |
| | putative | | | | | | |
| | protein | | | | | | |
| unknown function - | yfhF | BLi00876 | 1.23 | 1.65 | 2.40 | 3.02 | 3.01 |
| similar to cell-division | | | | | | | |
| inhibitor | | | | | | | |
| unknown function | gene for a | BLi01622 | 0.95 | 1.19 | 2.52 | 3.81 | 3.13 |
| | hypothetical | | | | | | |
| | protein | | | | | | |
| putative glycerol | gene for a | BLi00828 | 2.03 | 5.17 | 6.18 | 5.69 | 6.48 |
| dehydrogenase (EC | putative | | | | | | |
| 1.1.1.6) | protein | | | | | | |
| putative Na(+)-linked D- | gene for a | BLi00817 | 3.91 | 6.40 | 13.40 | 12.49 | 17.09 |
| alanine glycine | putative | | | | | | |
| permease | protein | | | | | | |
| unknown function - | ytcA | BLi00810 | 1.40 | 3.88 | 4.32 | 4.56 | 4.07 |
| similar to NDP-sugar | | | | | | | |
| dehydrogenase | | | | | | | |
| unknown function - | ymcA | BLi01926 | 1.14 | 2.26 | 2.58 | 2.13 | 3.69 |
| similar to unknown | | | | | | | |
| function proteins | | | | | | | |
| unknown function | gene for a | BLi01933 | 6.35 | 5.54 | 0.93 | 3.71 | 0.97 |
| | conserved | | | | | | |
| | hypothetical | | | | | | |
| | protein | | | | | | |
| NADH dehydrogenase | gene for a | BLi01934 | 6.56 | 4.40 | 0.95 | 3.87 | 0.98 |
| (EC 1.6.99.3) | NADH- | | | | | | |
| | ubiquinone | | | | | | |
| | oxidoreductase | | | | | | |
| | 51 kDa | | | | | | |
| | subunit (EC | | | | | | |
| | 1.6.5.3) | | | | | | |
| unknown function - | yjiC | BLi01948 | 1.23 | 3.15 | 1.89 | 3.04 | 1.56 |
| similar to macrolide | | | | | | | |
| glycosyl transferase | | | | | | | |
| gamma- | ggt | BLi01364 | 1.81 | 8.65 | 7.15 | 8.76 | 6.61 |
| glutamyltranspeptidase | | | | | | | |
| putative beta-lactamase | gene for a | BLi00328 | 1.12 | 1.52 | 3.83 | 5.64 | 5.50 |
| protein | putative | | | | | | |
| | protein | | | | | | |
| unknown function - | ysfD | BLi00329 | 0.95 | 1.18 | 3.09 | 3.14 | 3.84 |
| similar to glycolate | | | | | | | |
| oxidase subunit | | | | | | | |
| unknown function | ycdC | BLi00343 | 1.20 | 1.78 | 4.31 | 4.81 | 4.79 |
| putative transcriptional | gene for a | BLi00353 | 1.32 | 1.58 | 3.25 | 3.00 | 2.74 |
| regulatory protein | putative | | | | | | |
| | protein | | | | | | |
| unknown function - | yceC | BLi00354 | 2.21 | 3.05 | 4.99 | 4.72 | 4.73 |
| similar to tellurium | | | | | | | |
| resistance protein | | | | | | | |
| unknown function - | yceD | BLi00355 | 2.36 | 3.85 | 5.35 | 4.14 | 4.85 |
| similar to tellurium | | | | | | | |
| resistance protein | | | | | | | |
| unknown function - | yceE | BLi00356 | 2.09 | 3.65 | 5.92 | 5.42 | 7.25 |
| similar to tellurium | | | | | | | |
| resistance protein | | | | | | | |
| unknown function - | yceF | BLi00357 | 1.62 | 1.98 | 2.98 | 2.82 | 3.29 |
| similar to tellurium | | | | | | | |
| resistance protein | | | | | | | |
| nitrate transporter | nasA | BLi00365 | 1.65 | 2.15 | 3.28 | 2.26 | 2.73 |
| NH3-dependent NAD+ | nadE | BLi00370 | 1.51 | 3.29 | 3.25 | 2.75 | 2.47 |
| synthetase | | | | | | | |
| unknown function - | ycgL | BLi00372 | 1.44 | 2.98 | 3.02 | 2.14 | 1.72 |
| similar to unknown | | | | | | | |
| function proteins | | | | | | | |
| unknown function - | ycgM | BLi00373 | 1.17 | 10.73 | 14.51 | 18.05 | 2.61 |
| similar to proline | | | | | | | |
| oxidase | | | | | | | |
| unknown function - | ycgN | BLi00374 | 1.20 | 7.65 | 8.25 | 12.14 | 1.88 |
| similar to 1-pyrroline-5- | | | | | | | |
| carboxylate | | | | | | | |
| dehydrogenase | | | | | | | |
| unknown function - | ycgO | BLi00375 | 1.13 | 2.14 | 3.79 | 3.37 | 1.90 |
| similar to proline | | | | | | | |
| permease | | | | | | | |
| unknown function - | yqfA | BLi02729 | 1.33 | 1.75 | 3.91 | 4.88 | 5.84 |
| similar to unknown | | | | | | | |
| function proteins | | | | | | | |
| close homolog to HemH | gene for a | BLi04115 | 1.44 | 1.91 | 1.67 | 3.47 | 3.20 |
| ferrochelatase | hypothetical | | | | | | |
| | protein | | | | | | |
| unknown function | gene for a | BLi04116 | 1.66 | 2.25 | 1.84 | 7.40 | 8.31 |
| | hypothetical | | | | | | |
| | protein | | | | | | |
| putative ABC | gene for a | BLi04117 | 2.08 | 2.88 | 2.08 | 9.26 | 10.49 |
| transporter ATP-binding | putative | | | | | | |
| protein | protein | | | | | | |
| unknown function | gene for a | BLi04118 | 1.08 | 1.72 | 2.23 | 3.90 | 6.80 |
| | hypothetical | | | | | | |
| | protein | | | | | | |
| unknown function | gene for a | BLi04119 | 5.53 | 1.72 | 1.42 | 5.46 | 4.59 |
| | hypothetical | | | | | | |
| | protein | | | | | | |
| unknown function | gene for a | BLi04120 | 1.68 | 2.29 | 2.02 | 7.63 | 7.32 |
| | conserved | | | | | | |
| | hypothetical | | | | | | |
| | protein | | | | | | |
| unknown function | gene for a | BLi04124 | 1.19 | 2.46 | 3.94 | 4.59 | 6.77 |
| | conserved | | | | | | |
| | hypothetical | | | | | | |
| | protein | | | | | | |
| putative bacteriocin | gene for a | BLi04126 | 1.59 | 3.71 | 4.73 | 6.92 | 6.89 |
| formation protein | putative | | | | | | |
| | protein | | | | | | |
| putative bacteriocin | gene for a | BLi04128 | 1.49 | 2.73 | 3.80 | 4.16 | 6.01 |
| formation protein | putative | | | | | | |
| | protein | | | | | | |
| ABC transporter | cydD | BLi04131 | 1.17 | 1.89 | 2.07 | 3.27 | 3.26 |
| required for expression | | | | | | | |
| of cytochrome bd (ATP- | | | | | | | |
| binding protein) | | | | | | | |
| ABC transporter | cydC | BLi04132 | 1.19 | 2.33 | 1.80 | 4.73 | 3.59 |
| required for expression | | | | | | | |
| of cytochrome bd (ATP- | | | | | | | |
| binding protein) | | | | | | | |
| cytochrome bd | cydB | BLi04133 | 0.90 | 1.98 | 1.71 | 3.50 | 3.09 |
| ubiquinol oxidase | | | | | | | |
| (subunit II) | | | | | | | |
| cytochrome bd | cydA | BLi04134 | 0.92 | 2.44 | 2.15 | 4.48 | 3.32 |
| ubiquinol oxidase | | | | | | | |
| (subunit I) | | | | | | | |
| family serine protease, | clpP | BLi03615 | 2.62 | 4.39 | 3.50 | 2.56 | 1.85 |
| possible phage related | | | | | | | |
| unknown function - | yciC | BLi00765 | 9.94 | 5.08 | 0.41 | 9.70 | 0.44 |
| similar to unknown | | | | | | | |
| function proteins | | | | | | | |
| citrate synthase I | citA | BLi01010 | 1.10 | 1.81 | 3.51 | 5.78 | 8.09 |
| (minor) | | | | | | | |
| unknown function - | yueE | BLi03372 | 1.27 | 4.43 | 3.09 | 4.19 | 4.28 |
| similar to unknown | | | | | | | |
| function proteins | | | | | | | |
| threonyl-tRNA | thrZ | BLi00234 | 2.34 | 4.75 | 4.27 | 5.60 | 5.26 |
| synthetase (minor) | | | | | | | |
| ribose ABC transporter | rbsA | BLi03843 | 1.28 | 4.95 | 1.81 | 2.27 | 2.60 |
| (ATP-binding protein) | | | | | | | |
| ribose ABC transporter | rbsB | BLi03845 | 2.09 | 5.61 | 2.33 | 4.47 | 2.79 |
| (ribose-binding protein) | | | | | | | |
| alpha-acetolactate | alsD | BLi03847 | 5.84 | 22.15 | 7.42 | 38.12 | 9.31 |
| decarboxylase | | | | | | | |
| alpha-acetolactate | alsS | BLi03848 | 5.86 | 44.43 | - | 40.12 | 10.29 |
| synthase | | | 17.73 | 76.83 | 9.41 | 143.53 | 23.96 |
| transcriptional regulator | alsR | BLi03849 | 1.75 | 8.78 | 7.50 | 8.75 | 4.33 |
| of the alpha- | | | | | | | |
| acetolactate operon | | | | | | | |
| unknown function - | ywrD | BLi03850 | 2.28 | 5.04 | 3.15 | 4.71 | 3.15 |
| similar to gamma- | | | | | | | |
| glutamyl transferase | | | | | | | |
| unknown function - | ywrC | BLi03851 | 2.16 | 5.23 | 3.08 | 7.25 | 3.90 |
| similar to transcriptional | | | | | | | |
| regulator (Lrp/AsnC | | | | | | | |
| family) | | | | | | | |
| unknown function - | ywrB | BLi03852 | 1.37 | 3.18 | 2.18 | 3.40 | 2.41 |
| similar to chromate | | | | | | | |
| transport protein | | | | | | | |
| unknown function - | ywqE | BLi03854 | 1.45 | 3.05 | 2.83 | 3.77 | 2.59 |
| similar to capsular | | | | | | | |
| polysaccharide | | | | | | | |
| biosynthesis | | | | | | | |
| unknown function - | ywqC | BLi03855 | 1.44 | 3.43 | 2.96 | 4.29 | 2.82 |
| similar to capsular | | | | | | | |
| polysaccharide | | | | | | | |
| biosynthesis | | | | | | | |
| unknown function | gene for a | BLi03108 | 1.72 | 3.11 | 1.60 | 6.93 | 2.50 |
| | conserved | | | | | | |
| | hypothetical | | | | | | |
| | protein | | | | | | |
| putative transcriptional | gene for a | BLi03548 | 1.15 | 3.37 | 3.93 | 5.48 | 3.52 |
| regulator | putative | | | | | | |
| | protein | | | | | | |
| unknown function - | yabE | BLi00053 | 0.83 | 1.43 | 1.70 | 3.29 | 3.20 |
| similar to cell wall- | | | | | | | |
| binding protein | | | | | | | |
| modulator of CtsR | mcsB | BLi00103 | 1.84 | 3.21 | 3.40 | 2.32 | 1.97 |
| repression | | | | | | | |
| class III stress | clpC | BLi00104 | 2.29 | 5.57 | 4.44 | 2.80 | 2.49 |
| response-related | | | | | | | |
| ATPase | | | | | | | |
| RNA polymerase | sigH | BLi00116 | 1.52 | 2.67 | 2.74 | 2.97 | 3.04 |
| sigma-H factor (sigma- | | | | | | | |
| 30) | | | | | | | |
| putative DNA-binding | gene for a | BLi04317 | 1.67 | 3.09 | 4.94 | 2.87 | 2.53 |
| protein | putative | | | | | | |
| | protein | | | | | | |
| putative am | gene for a | BLi01175 | 6.20 | 19.95 | 19.06 | 20.96 | 18.99 |
| | putative | | | | | | |
| | protein | | | | | | |
| asparagine synthetase | asnO | BLi01174 | 2.16 | 5.61 | 5.19 | 5.46 | 5.89 |
| signal peptidase I | sipS | BLi00675 | 1.24 | 2.55 | 2.48 | 3.30 | 3.28 |
| unknown function | yeaA | BLi00672 | 1.21 | 3.04 | 3.59 | 5.20 | 4.83 |
| unknown function | gene for a | BLi00669 | 1.16 | 2.35 | 3.72 | 6.32 | 4.27 |
| | hypothetical | | | | | | |
| | protein | | | | | | |
| unknown function | gene for a | BLi00668 | 1.22 | 3.68 | 4.32 | 7.18 | 4.47 |
| | hypothetical | | | | | | |
| | protein | | | | | | |
| unknown function - | ywaE | BLi00667 | 1.18 | 2.16 | 2.87 | 3.47 | 4.12 |
| similar to transcriptional | | | | | | | |
| regulator (MarR family) | | | | | | | |
| unknown function - | yvdH | BLi00660 | 1.13 | 1.96 | 2.31 | 3.45 | 3.27 |
| similar to maltodextrin | | | | | | | |
| transport system | | | | | | | |
| permease | | | | | | | |
| unknown function - | ycdH | BLi03213 | 21.12 | 7.72 | 1.98 | 4.50 | 1.00 |
| similar to ABC | | | | | | | |
| transporter (binding | | | | | | | |
| protein) | | | | | | | |
| anthranilate synthase | trpE | BLi02403 | 1.96 | 26.10 | 22.29 | 19.75 | 23.14 |
| anthranilate | trpD | BLi02402 | 1.50 | 10.22 | 13.01 | 11.88 | 13.42 |
| phosphoribosyl | | | | | | | |
| transferase | | | | | | | |
| indol-3-glycerol | trpC | BLi02401 | 1.50 | 12.08 | 14.43 | 11.15 | 15.42 |
| phosphate synthase | | | | | | | |
| phosphoribosyl | trpF | BLi02400 | 1.43 | 6.15 | 12.79 | 11.61 | 12.06 |
| anthranilate isomerase | | | | | | | |
| tryptophan synthase | trpB | BLi02399 | 1.50 | 9.20 | 13.19 | 11.39 | 14.53 |
| (beta subunit) | | | | | | | |
| tryptophan synthase | trpA | BLi02398 | 1.03 | 5.97 | 7.01 | 5.26 | 7.49 |
| (alpha subunit) | | | | | | | |
| histidinol-phosphate | hisC | BLi02397 | 1.56 | 6.27 | 5.16 | 5.05 | 5.96 |
| amino transferase/ | | | | | | | |
| tyrosine and | | | | | | | |
| phenylalanine amino | | | | | | | |
| transferase | | | | | | | |
| prephenate | tyrA | BLi02396 | 1.07 | 2.31 | 4.17 | 4.00 | 3.06 |
| dehydrogenase | | | | | | | |
| 5- | aroE | BLi02395 | 1.14 | 3.90 | 4.02 | 3.61 | 3.89 |
| enolpyruvoylshikimate- | | | | | | | |
| 3-phosphate synthase | | | | | | | |
| unknown function - | ypiB | BLi02393 | 1.69 | 5.02 | 5.60 | 6.15 | 6.43 |
| similar to unknown | | | | | | | |
| function proteins | | | | | | | |
| unknown function - | yqeH | BLi02760 | 1.01 | 2.79 | 2.99 | 3.56 | 3.55 |
| similar to unknown | | | | | | | |
| function proteins | | | | | | | |
| unknown function | yqeF | BLi02763 | 1.08 | 2.86 | 3.45 | 3.56 | 3.28 |
| two-component | dctR | BLi02781 | 1.17 | 4.50 | 4.05 | 4.44 | 3.82 |
| response regulator | | | | | | | |
| involved in C4- | | | | | | | |
| dicarboxylate transport | | | | | | | |
| putative two-component | gene for a | BLi02787 | 1.19 | 5.14 | 2.62 | 4.67 | 2.86 |
| response regulator | putative | | | | | | |
| involved in degradative | protein | | | | | | |
| enzyme and | | | | | | | |
| competence regulation | | | | | | | |
| 6-phospho-3- | hxlB | BLi02804 | 1.25 | 3.15 | 1.35 | 2.56 | 2.48 |
| hexuloisomerase | | | | | | | |
| 3-hexulose-6-phosphate | hxlA | BLi02805 | 2.56 | 4.13 | 2.48 | 3.73 | 2.10 |
| synthase | | | | | | | |
| unknown function | yppF | BLi02362 | 1.15 | 3.31 | 2.69 | 4.07 | 8.62 |
| alkyl hydroperoxide | ahpF | BLi04292 | 2.30 | 3.07 | 2.33 | 2.55 | 2.10 |
| reductase (large | | | | | | | |
| subunit)/NADH | | | | | | | |
| dehydrogenase | | | | | | | |
| alkyl hydroperoxide | ahpC | BLi04291 | 3.17 | 3.21 | 2.35 | 2.40 | 1.50 |
| reductase (small | | | | | | | |
| subunit) | | | | | | | |
| putative | gene for a | BLi04251 | 1.09 | 2.25 | 2.06 | 3.02 | 2.97 |
| methylmalonate- | putative | | | | | | |
| semialdehyde | protein | | | | | | |
| dehydrogenase | | | | | | | |
| [acylating] (EC 1.2.1.27) | | | | | | | |
| unknown function - | ykoU | BLi01494 | 1.91 | 5.27 | 5.01 | 5.64 | 6.04 |
| similar to ATP- | | | | | | | |
| dependent DNA ligase | | | | | | | |
| unknown function - | ykoM | BLi01493 | 2.13 | 5.84 | 6.31 | 7.24 | 5.83 |
| similar to transcriptional | | | | | | | |
| regulator (MarR family) | | | | | | | |
| unknown function - | ydfS | BLi01489 | 2.03 | 6.03 | 4.84 | 13.04 | 6.72 |
| similar to unknown | | | | | | | |
| function proteins | | | | | | | |
| unknown function | gene for a | BLi01488 | 7.88 | 73.93 | 103.66 | 146.73 | 54.19 |
| | hypothetical | | | | | | |
| | protein | | | | | | |
| unknown function - | yrkC | BLi01487 | 3.01 | 38.73 | 35.62 | 62.29 | 20.60 |
| similar to unknown | | | | | | | |
| function proteins | | | | | | | |
| putative glycosyl | gene for a | BLi01486 | 2.19 | 8.60 | 10.08 | 16.48 | 13.52 |
| hydrolase/lysozyme | putative | | | | | | |
| | protein | | | | | | |
| unknown function | yncE | BLi03150 | 1.84 | 5.91 | 7.32 | 11.97 | 10.11 |
| unknown function - | yhbD | BLi00958 | 1.45 | 5.23 | 7.84 | 17.05 | 12.08 |
| similar to unknown | | | | | | | |
| function proteins from | | | | | | | |
| B. subtilis | | | | | | | |
| unknown function - | yvlB | BLi03752 | 1.45 | 3.88 | 7.43 | 10.22 | 12.00 |
| similar to unknown | | | | | | | |
| function proteins | | | | | | | |
| unknown function - | yvlD | BLi03750 | 0.93 | 1.31 | 2.96 | 4.00 | 4.49 |
| similar to unknown | | | | | | | |
| function proteins | | | | | | | |
| unknown function - | yxkH | BLi04151 | 0.77 | 1.67 | 4.79 | 5.99 | 5.82 |
| similar to unknown | | | | | | | |
| function proteins | | | | | | | |
| transcriptional regulator | pucR | BLi03436 | 1.91 | 2.82 | 4.27 | 4.96 | 4.20 |
| of puc genes | | | | | | | |
| transcriptional activator | gltC | BLi02163 | 1.15 | 3.54 | 4.43 | 5.21 | 5.34 |
| of the glutamate | | | | | | | |
| synthase operon | | | | | | | |
| unknown function | gene for a | BLi03570 | 2.58 | 3.81 | 6.18 | 5.44 | 5.03 |
| | hypothetical | | | | | | |
| | protein | | | | | | |
| putative amidase | gene for a | BLi03571 | 1.95 | 2.40 | 2.11 | 3.45 | 2.96 |
| | putative | | | | | | |
| | protein | | | | | | |
| unknown function - | ylbP | BLi01727 | 1.56 | 4.57 | 3.28 | 3.27 | 2.35 |
| similar to unknown | | | | | | | |
| function proteins | | | | | | | |
| putative ABC | gene for a | BLi03212 | 8.10 | 4.42 | 1.28 | 4.52 | 1.45 |
| transporter/amino acid | putative | | | | | | |
| permease | protein | | | | | | |
| putative nitrogen | gene for a | BLi01176 | 8.33 | 30.73 | 31.25 | 33.74 | 30.23 |
| regulatory protein P-II | putative | | | | | | |
| | protein | | | | | | |
| unknown function | gene for a | BLi00719 | 6.47 | 17.69 | 14.11 | 18.09 | 17.23 |
| | hypothetical | | | | | | |
| | protein | | | | | | |
| unknown function - | ywpF | BLi03871 | 1.51 | 3.86 | 3.82 | 5.12 | 3.69 |
| similar to unknown | | | | | | | |
| function proteins | | | | | | | |
| unknown function | yvlA | BLi03753 | 1.32 | 4.63 | 8.68 | 10.55 | 11.65 |
| unknown function - | ywbD | BLi04060 | 2.49 | 2.17 | 3.88 | 6.12 | 4.20 |
| similar to unknown | | | | | | | |
| function proteins | | | | | | | |
| unknown function | gene for a | BLi00654 | 1.06 | 6.05 | 3.60 | 4.45 | 3.81 |
| | conserved | | | | | | |
| | hypothetical | | | | | | |
| | protein | | | | | | |
| putative phage tail | gene for a | BLi01476 | 1.01 | 4.36 | 2.24 | 3.31 | 2.37 |
| protein | putative | | | | | | |
| | protein | | | | | | |
| peptidyl methionine | msrA | BLi02303 | 1.83 | 4.46 | 3.66 | 3.71 | 2.74 |
| sulfoxide reductase | | | | | | | |
| unknown function | gene for a | BLi00235 | 5.03 | 10.82 | 7.36 | 11.39 | 9.29 |
| | hypothetical | | | | | | |
| | protein | | | | | | |

### Example 3

### Genes that are Markedly Induced Specifically Under Nitrogen Deficiency

As is shown in Table 1, a total of 67 of all the Bacillus licheniformis DSM13 genes investigated in Example 1 under the conditions of nitrogen deficiency were induced by at least a factor of 8 at any of the measured times. Among these were also 18 genes that were also induced by the deficiency of at least one other compound (data not shown) and which therefore cannot be regarded as specific signals for a nitrogen deficiency.

Consequently there remain 49 genes that were induced by at least a factor 8 at any of the measured times and may be classified as comparatively specific. They are compiled in Table 2 below. The column headers are the same as in the preceding example. In addition, the first column indicates the sequence numbers of the particular DNA and amino acid sequences in the sequence listing of the present application. Specific features of the particular sequences, which appear as free text in the sequence listing have been added under the heading Gene name/gene function.

TABLE 2
The 62 Bacillus licheniformis DSM13 genes determined in Example
1, whose specific induction (by at least a factor of 8) under nitrogen
deficiency was observed at any of the measured times
(explanations: see text).

| SEQ | Protein name/ | | | | | | | |
|---|---|---|---|---|---|---|---|---|
| ID | Gene function/ | | | | | | | |
| NO. | Notes on the gene | Gene | BLi—Nr | Transition | 30 min | 60 min | 90 min | 120 min |
| 1, 2 | unknown function - | yqjN | BLi02550 | 1.56 | 2.68 | 4.48 | 8.50 | 8.65 |
| | similar to amino | | | | | | | |
| | acid degradation | | | | | | | |
| | The first codon is | | | | | | | |
| | translated as | | | | | | | |
| | methionine. | | | | | | | |
| 3, 4 | nitrogen-regulated | nrgB | BLi03889 | 3.61 | 6.92 | 5.04 | 9.99 | 6.67 |
| | PII-like protein | | | | | | | |
| 5, 6 | ammonium | nrgA | BLi03888 | 3.04 | 5.71 | 3.91 | 9.56 | 7.31 |
| | transporter | | | | | | | |
| 7, 8 | serine protease Do | htrA | BLi01390 | 7.91 | 8.10 | 2.03 | 1.93 | 1.67 |
| | (heat-shock protein) | | | | | | | |
| 9, 10 | putative serine | gene for a | BLi00301 | 2.31 | 13.40 | 7.51 | 15.91 | 11.55 |
| | protease | putative | | | | | | |
| | The first codon is | protein | | | | | | |
| | translated as | | | | | | | |
| | methionine. | | | | | | | |
| 11, | transcriptional | glnR | BLi01992 | 4.38 | 11.07 | 7.61 | 9.77 | 8.25 |
| 12 | repressor of the | | | | | | | |
| | glutamine | | | | | | | |
| | synthetase gene | | | | | | | |
| 13, | glutamine | glnA | BLi01993 | 5.69 | 9.51 | 8.60 | 9.06 | 8.61 |
| 14 | synthetase | | | | | | | |
| 15, | close homolog to | gene for a | BLi03994 | 5.33 | 9.09 | 10.32 | 22.37 | 19.12 |
| 16 | DhaS aldehyde | hypothetical | | | | | | |
| | dehydrogenase | protein | | | | | | |
| 17, | putative | gene for a | BLi03995 | 1.82 | 5.01 | 7.61 | 12.47 | 15.76 |
| 18 | transcriptional | putative | | | | | | |
| | regulator | protein | | | | | | |
| 19, | unknown function | gene for a | BLi03996 | 1.52 | 2.34 | 4.24 | 8.76 | 10.13 |
| 20 | The first codon is | conserved | | | | | | |
| | translated as | hypothetical | | | | | | |
| | methionine. | protein | | | | | | |
| 21, | putative phage | gene for a | BLi01470 | 3.21 | 14.00 | 9.43 | 9.07 | 5.59 |
| 22 | capsid protein | putative | | | | | | |
| | | protein | | | | | | |
| 23, | unknown function - | yvtA | BLi03481 | 9.99 | 3.41 | 1.30 | 1.61 | 1.00 |
| 24 | similar to HtrA-like | | | | | | | |
| | serine protease | | | | | | | |
| 25, | transcriptional | kdgR | BLi03828 | 0.77 | 1.99 | 2.15 | 4.81 | 8.02 |
| 26 | repressor of the | | | | | | | |
| | pectin utilization | | | | | | | |
| | operon | | | | | | | |
| 27, | unknown function - | ycnI | BLi00479 | 3.74 | 5.12 | 3.84 | 8.23 | 3.18 |
| 28 | similar to unknown | | | | | | | |
| | function proteins | | | | | | | |
| 29, | unknown function - | ycnJ | BLi00480 | 4.04 | 5.28 | 4.59 | 10.74 | 3.79 |
| 30 | similar to copper | | | | | | | |
| | export protein | | | | | | | |
| 31, | unknown function - | ycnK | BLi00481 | 4.60 | 10.14 | 8.17 | 14.46 | 8.30 |
| 32 | similar to | | | | | | | |
| | transcriptional | | | | | | | |
| | regulator (DeoR | | | | | | | |
| | family) | | | | | | | |
| 33, | assimilatory nitrate | nasB | BLi00482 | 18.17 | 22.63 | 20.62 | 25.24 | 16.47 |
| 34 | reductase (electron | | | | | | | |
| | transfer subunit) | | | | | | | |
| 35, | assimilatory nitrate | nasC | BLi00483 | 11.07 | 22.08 | 20.33 | 21.01 | 20.53 |
| 36 | reductase (catalytic | | | | | | | |
| | subunit) | | | | | | | |
| 37, | assimilatory nitrite | nasD | BLi00484 | 6.63 | 10.58 | 15.49 | 20.64 | 12.06 |
| 38 | reductase (subunit) | | | | | | | |
| | The first codon is | | | | | | | |
| | translated as | | | | | | | |
| | methionine. | | | | | | | |
| 39, | uroporphyrin-III C- | nasF | BLi00486 | 8.08 | 37.38 | 22.44 | 36.50 | 18.60 |
| 40 | methyltransferase | | | | | | | |
| 41, | unknown function | gene for a | BLi00236 | 2.98 | 5.18 | 6.04 | 7.59 | 8.29 |
| 42 | | hypothetical | | | | | | |
| | | protein | | | | | | |
| 43, | phosphoenolpyruvate | pckA | BLi03197 | 0.90 | 5.90 | 7.35 | 28.58 | 10.77 |
| 44 | carboxykinase | | | | | | | |
| 45, | putative malate | gene for a | BLi04208 | 1.00 | 5.95 | 3.71 | 16.98 | 2.98 |
| 46 | synthase (EC | putative | | | | | | |
| | 4.1.3.2) | protein | | | | | | |
| 47, | putative hydrolase | gene for a | BLi02233 | 1.29 | 3.38 | 8.50 | 12.34 | 6.57 |
| 48 | | putative | | | | | | |
| | | protein | | | | | | |
| 49, | putative Na(+)- | gene for a | BLi00817 | 3.91 | 6.40 | 13.40 | 12.49 | 17.09 |
| 50 | linked D-alanine | putative | | | | | | |
| | glycine permease | protein | | | | | | |
| 51, | gamma- | ggt | BLi01364 | 1.81 | 8.65 | 7.15 | 8.76 | 6.61 |
| 52 | glutamyltranspeptidase | | | | | | | |
| 53, | unknown function | gene for a | BLi04116 | 1.66 | 2.25 | 1.84 | 7.40 | 8.31 |
| 54 | | hypothetical | | | | | | |
| | | protein | | | | | | |
| 55, | putative ABC | gene for a | BLi04117 | 2.08 | 2.88 | 2.08 | 9.26 | 10.49 |
| 56 | transporter ATP- | putative | | | | | | |
| | binding protein | protein | | | | | | |
| 57, | unknown function - | yciC | BLi00765 | 9.94 | 5.08 | 0.41 | 9.70 | 0.44 |
| 58 | similar to unknown | | | | | | | |
| | function proteins | | | | | | | |
| 59, | citrate synthase I | citA | BLi01010 | 1.10 | 1.81 | 3.51 | 5.78 | 8.09 |
| 60 | (minor) | | | | | | | |
| 61, | transcriptional | alsR | BLi03849 | 1.75 | 8.78 | 7.50 | 8.75 | 4.33 |
| 62 | regulator of the | | | | | | | |
| | alpha-acetolactate | | | | | | | |
| | operon | | | | | | | |
| 63, | putative ammonium | gene for a | BLi01175 | 6.20 | 19.95 | 19.06 | 20.96 | 18.99 |
| 64 | transporter | putative | | | | | | |
| | | protein | | | | | | |
| 65, | unknown function - | ycdH | BLi03213 | 21.12 | 7.72 | 1.98 | 4.50 | 1.00 |
| 66 | similar to ABC | | | | | | | |
| | transporter (binding | | | | | | | |
| | protein) | | | | | | | |
| 67, | anthranilate | trpE | BLi02403 | 1.96 | 26.10 | 22.29 | 19.75 | 23.14 |
| 68 | synthase | | | | | | | |
| | The first codon is | | | | | | | |
| | translated as | | | | | | | |
| | methionine. | | | | | | | |
| 69, | anthranilate | trpD | BLi02402 | 1.50 | 10.22 | 13.01 | 11.88 | 13.42 |
| 70 | phosphoribosyl | | | | | | | |
| | transferase | | | | | | | |
| 71, | indol-3-glycerol | trpC | BLi02401 | 1.50 | 12.08 | 14.43 | 11.15 | 15.42 |
| 72 | phosphate synthase | | | | | | | |
| 73, | phosphoribosyl | trpF | BLi02400 | 1.43 | 6.15 | 12.79 | 11.61 | 12.06 |
| 74 | anthranilate | | | | | | | |
| | isomerase | | | | | | | |
| 75, | tryptophan synthase | trpB | BLi02399 | 1.50 | 9.20 | 13.19 | 11.39 | 14.53 |
| 76 | (beta subunit)) | | | | | | | |
| 77, | unknown function | yppF | BLi02362 | 1.15 | 3.31 | 2.69 | 4.07 | 8.62 |
| 78 | | | | | | | | |
| 79, | unknown function - | ydfS | BLi01489 | 2.03 | 6.03 | 4.84 | 13.04 | 6.72 |
| 80 | similar to unknown | | | | | | | |
| | function proteins | | | | | | | |
| 81, | unknown function | gene for a | BLi01488 | 7.88 | 73.93 | 103.66 | 146.73 | 54.19 |
| 82 | | hypothetical | | | | | | |
| | | protein | | | | | | |
| 83, | unknown function - | yrkC | BLi01487 | 3.01 | 38.73 | 35.62 | 62.29 | 20.60 |
| 84 | similar to unknown | | | | | | | |
| | function proteins | | | | | | | |
| 85, | putative glycosyl | gene for a | BLi01486 | 2.19 | 8.60 | 10.08 | 16.48 | 13.52 |
| 86 | hydrolase/ | putative | | | | | | |
| | lysozyme | protein | | | | | | |
| 87, | unknown function | yncE | BLi03150 | 1.84 | 5.91 | 7.32 | 11.97 | 10.11 |
| 88 | | | | | | | | |
| 89, | unknown function - | yvlB | BLi03752 | 1.45 | 3.88 | 7.43 | 10.22 | 12.00 |
| 90 | similar to unknown | | | | | | | |
| | function proteins | | | | | | | |
| 91, | putative ABC | gene for a | BLi03212 | 8.10 | 4.42 | 1.28 | 4.52 | 1.45 |
| 92 | transporter/amino | putative | | | | | | |
| | acid permease | protein | | | | | | |
| 93, | putative nitrogen | gene for a | BLi01176 | 8.33 | 30.73 | 31.25 | 33.74 | 30.23 |
| 94 | regulatory protein | putative | | | | | | |
| | P-II | protein | | | | | | |
| | The first codon is | | | | | | | |
| | translated as | | | | | | | |
| | methionine. | | | | | | | |
| 95, | unknown function | gene for a | BLi00719 | 6.47 | 17.69 | 14.11 | 18.09 | 17.23 |
| 96 | The first codon is | hypothetical | | | | | | |
| | translated as | protein | | | | | | |
| | methionine. | | | | | | | |
| 97, | unknown function | yvlA | BLi03753 | 1.32 | 4.63 | 8.68 | 10.55 | 11.65 |
| 98 | | | | | | | | |

In Table 3 below are listed the same 49 genes once more in the order of observed strength of induction. The corresponding terms in English are indicated in this order in the description section. The arrangement of the subject matter of the claims follows the reverse order.

TABLE 3
The 49 Bacillus licheniformis DSM13 genes determined in Example
1, whose specific induction (by at least a factor of 8) under nitrogen
deficiency was observed at any of the measured times, are listed in
descending order of the maximum value (last column) measured in
each case.

| SEQ | | | | |
|---|---|---|---|---|
| ID | Protein name/Gene function/ | | | |
| NO. | Notes on the gene | Gene | BLi-Nr | max. |
| 81, | unknown function | gene for a | BLi01488 | 146.73 |
| 82 | | hypothetical protein | | |
| 83, | unknown function - similar to | yrkC | BLi01487 | 62.29 |
| 84 | unknown function proteins | | | |
| 39, | uroporphyrin-III C-methyltransferase | nasF | BLi00486 | 37.38 |
| 40 | | | | |
| 93, | putative nitrogen regulatory protein P- | gene for a putative | BLi01176 | 33.74 |
| 94 | II | protein | | |
| | The first codon is translated as | | | |
| | methionine. | | | |
| 43, | phosphoenolpyruvate carboxykinase | pckA | BLi03197 | 28.58 |
| 44 | | | | |
| 67, | anthranilate synthase | trpE | BLi02403 | 26.10 |
| 68 | The first codon is translated as | | | |
| | methionine. | | | |
| 33, | assimilatory nitrate reductase | nasB | BLi00482 | 25.24 |
| 34 | (electron transfer subunit) | | | |
| 15, | close homolog to DhaS aldehyde | gene for a | BLi03994 | 22.37 |
| 16 | dehydrogenase | hypothetical protein | | |
| 35, | assimilatory nitrate reductase | nasC | BLi00483 | 22.08 |
| 36 | (catalytic subunit) | | | |
| 65, | unknown function - similar to ABC | ycdH | BLi03213 | 21.12 |
| 66 | transporter (binding protein) | | | |
| 63, | putative ammonium transporter | gene for a putative | BLi01175 | 20.96 |
| 64 | | protein | | |
| 37, | assimilatory nitrite reductase | nasD | BLi00484 | 20.64 |
| 38 | (subunit) | | | |
| | The first codon is translated as | | | |
| | methionine. | | | |
| 95, | unknown function | gene for a | BLi00719 | 18.09 |
| 96 | The first codon is translated as | hypothetical protein | | |
| | methionine. | | | |
| 49, | putative Na(+)-linked D-alanine | gene for a putative | BLi00817 | 17.09 |
| 50 | glycine permease | protein | | |
| 45, | putative malate synthase (EC 4.1.3.2) | gene for a putative | BLi04208 | 16.98 |
| 46 | | protein | | |
| 85, | putative glycosyl hydrolase/ | gene for a putative | BLi01486 | 16.48 |
| 86 | lysozyme | protein | | |
| 9, | putative serine protease | gene for a putative | BLi00301 | 15.91 |
| 10 | The first codon is translated as | protein | | |
| | methionine. | | | |
| 17, | putative transcriptional regulator | gene for a putative | BLi03995 | 15.76 |
| 18 | | protein | | |
| 71, | indol-3-glycerol phosphate synthase | trpC | BLi02401 | 15.42 |
| 72 | | | | |
| 75, | tryptophan synthase (beta subunit) | trpB | BLi02399 | 14.53 |
| 76 | | | | |
| 31, | unknown function - similar to | ycnK | BLi00481 | 14.46 |
| 32 | transcriptional regulator (DeoR | | | |
| | family) | | | |
| 21, | putative phage capsid protein | gene for a putative | BLi01470 | 14.00 |
| 22 | | protein | | |
| 69, | anthranilate phosphoribosyl | trpD | BLi02402 | 13.42 |
| 70 | transferase | | | |
| 79, | unknown function - similar to | ydfS | BLi01489 | 13.04 |
| 80 | unknown function proteins | | | |
| 73, | phosphoribosyl anthranilate | trpF | BLi02400 | 12.79 |
| 74 | isomerase | | | |
| 47, | putative hydrolase | gene for a putative | BLi02233 | 12.34 |
| 48 | | protein | | |
| 89, | unknown function - similar to | yvlB | BLi03752 | 12.00 |
| 90 | unknown function proteins | | | |
| 87, | unknown function | yncE | BLi03150 | 11.97 |
| 88 | | | | |
| 97, | unknown function | yvlA | BLi03753 | 11.65 |
| 98 | | | | |
| 11, | transcriptional repressor of the | glnR | BLi01992 | 11.07 |
| 12 | glutamine synthetase gene | | | |
| 29, | unknown function - similar to copper | ycnJ | BLi00480 | 10.74 |
| 30 | export protein | | | |
| 55, | putative ABC transporter ATP-binding | gene for a putative | BLi04117 | 10.49 |
| 56 | protein | protein | | |
| 19, | unknown function | gene for a | BLi03996 | 10.13 |
| 20 | The first codon is translated as | conserved | | |
| | methionine. | hypothetical protein | | |
| 3, 4 | nitrogen-regulated PII-like protein | nrgB | BLi03889 | 9.99 |
| 23, | unknown function - similar to HtrA- | yvtA | BLi03481 | 9.99 |
| 24 | like serine protease | | | |
| 57, | unknown function - similar to | yciC | BLi00765 | 9.94 |
| 58 | unknown function proteins | | | |
| 5, 6 | ammonium transporter | nrgA | BLi03888 | 9.56 |
| 13, | glutamine synthetase | glnA | BLi01993 | 9.51 |
| 14 | | | | |
| 61, | transcriptional regulator of the alpha- | alsR | BLi03849 | 8.78 |
| 62 | acetolactate operon | | | |
| 51, | gamma-glutamyltranspeptidase | ggt | BLi01364 | 8.76 |
| 52 | | | | |
| 1, 2 | unknown function - similar to amino | yqjN | BLi02550 | 8.65 |
| | acid degradation | | | |
| | The first codon is translated as | | | |
| | methionine. | | | |
| 77, | unknown function | yppF | BLi02362 | 8.62 |
| 78 | | | | |
| 53, | unknown function | gene for a | BLi04116 | 8.31 |
| 54 | | hypothetical protein | | |
| 41, | unknown function | gene for a | BLi00236 | 8.29 |
| 42 | | hypothetical protein | | |
| 27, | unknown function - similar to | ycnI | BLi00479 | 8.23 |
| 28 | unknown function proteins | | | |
| 7, 8 | serine protease Do (heat-shock | htrA | BLi01390 | 8.10 |
| | protein) | | | |
| 91, | putative ABC transporter/amino acid | gene for a putative | BLi03212 | 8.10 |
| 92 | permease | protein | | |
| 59, | citrate synthase I (minor) | citA | BLi01010 | 8.09 |
| 60 | | | | |
| 25, | transcriptional repressor of the pectin | kdgR | BLi03828 | 8.02 |
| 26 | utilization operon | | | |

### Example 4

### Real Time RT-PCR Quantification of Selected Genes

Real time RT-PCR enables the absolute numbers of molecules of specific transcripts in a sample to be determined. The LightCycler (Roche Diagnostics, Penzberg, Germany) was utilized in combination with the “SYBR Green I” kit (Roche Diagnostics). This method is based on detecting the binding of the fluorescent dye to double-stranded DNA. The specific mRNA molecules were quantified as described in Tobisch et al. (2003; Quantification of Bacterial mRNA by One-Step RT-PCR Using the LightCycler System; BIOCHEMICA, Volume 3, pages 5 to 8), with an external standard curve being established for each mRNA to be studied.

For this purpose, dilutions of known concentrations of in vitro transcripts of the mRNAs determined in Example 1 and listed in Example 2 were measured with the aid of the LightCycler, and then the standard curve was generated using the apparatus-specific software. For this purpose, primers, which are specific for each mRNA to be studied, and which enable in vitro transcripts to be synthesized and PCR amplification in the LightCycler had to be selected beforehand (with the aid of the “Array Designer” software; available from PREMIER Biosoft International, Palo Alto, USA).

After generating the external standard curve, measurement in the LightCycler was begun. Two different dilutions of each sample to be analyzed were used for the measurements. The particular primers were then used in the LightCycler run to amplify the specific transcript, and the incorporation of the dye was then measured.

Using the LightCycler software and the script accessible on the website http://molbiol.ru/ger/scripts/01_{—}07.html (12.2.2004), it was possible to determine the exact number of molecules for selected transcripts. The values obtained in this manner and their relations to one another were then used to confirm the induction values indicated in Tables 1 and 2 for the selected transcripts.

### Example 5

### Real Time RT-PCR Quantification of the Genes glnA, nrgB and tnrA

The genes glnA (SEQ ID NO. 13), nrgB (SEQ ID NO. 3) and tnrA were subjected to an additional Real-Time-RT-PCR, as described in the previous example. tnrA concerns the transcriptional regulator involved in global nitrogen regulation gene illustrated in SEQ ID NO. 99 (Data in the sequence listing: <223> tnrA—transcriptional pleiotropic regulator involved in global nitrogen regulation—BLi 01490). This gene incorporating 333 by is obtainable from B. licheniformis DSM 13 and is recorded in the abovementioned databank under the number BLi 01490. The derived amino acid sequence is listed under SEQ ID NO. 100.

The measurements were again carried out with the LightCycler instrument using the SYBR Green detection kit. The gene-specific primers were derived with the Programm Array Designer 2.0 (PREMIER Biosoft International, Palo Alto, USA) and ordered from Invitrogen (Karlsruhe, Germany). For the quantification of the specific mRNA, an external standard curve was generated with the help of an in-vitro transcript. The in-vitro transcript was constructed using a T7 polymerase, the gene-specific PCR product and the DIG-RNA-Labeling-Kit (Roche Diagnostics, Penzberg, Germany). The concentration and quality of the in-vitro transcript were determined photometrically.

A dilution series (1 ng to 100 fg final concentration) of the in-vitro transcript was prepared and mixed with MS2-RNA (final concentration 0.5 μg/μl; Roche Diagnostics) the forward and reverse primers and the LightCycler-RNA-Master-SYBR-Green I-Kit (Roche Diagnostics) as the mastermix, according to the manufacturer's instructions. The standard curve for the specific gene was generated by means of the manufacturer's notes for the RNA-Master-SYBRGreen I-Kit (amplification, segment 2, temperature: 57° C.; amplification, segment 3, incubation time: 16 s). The result of the LightCycler run was evaluated with the help of the LightCycler-Software and the Second-derivative-maximum Method, and the quality of the RT-PCR products was checked by the melting point determination.

For the measurements of the RNA samples, dilution steps from 500 ng to 5 ng of the total RNA were prepared and mixed with MS2-RNA (final concentration 0.5 μg/μl), the specific primer pairs and the LightCycler-RNA-Master-SYBR-Green I-Kit. The same conditions that were also used with the LightCycler instrument for generating the standard curve were used for the Real-time-RT-PCR run.

The negative control was RNase-free water with MS2-RNA in a final concentration of 0.5 μg/μl. A selected dilution of a sample from the standard curve was carried out in each case during the LightCycler run.

The LightCycler measurements were evaluated with the LightCycler-specific software. The molecular quantities of the specific RNA in the measured sample were determined with the help of the standard curve and the Second derivative maximum method.

The results compiled in the Table 4 below refer in each case to the molecular quantities during the logarithmic phase, that in each case were set to 1 (control). Samples were taken at the time of onset of the nitrogen limitation that is linked, as described above, with the transition into the stationary phase (transient phase), as well as 30, 60 and 120 minutes after this transition.

TABLE 4
Real-Time-RT-PCR-Quantification of the Genes glnA,
nrgB and tnrA with the onset of a nitrogen deficiency and
the consequent transition into the stationary phase

| | glnA | nrgB | tnrA |
|---|---|---|---|
| Control | 1 | 1 | 1 |
| Transient Phase | 11.5 | 15 | 10.65 |
| 30 min | 12.5 | 14.25 | 15.24 |
| stationary | | | |
| 60 min | 5.5 | 5.75 | 9,375 |
| stationary | | | |
| 120 min | 3.3 | 4,875 | 8.85 |
| stationary | | | |

It can be seen that in this measurement, all three genes glnA, nrgB and tnrA are strongly expressed at the onset of a nitrogen deficiency and the transition into the stationary phase linked to this, in the case of tnrA are expressed up to 15.24 fold greater than during the exponential growth phase and the associated optimal nitrogen supply. Consequently, they are suitable inventive marker genes for indicating an onset of nitrogen deficiency.

### DESCRIPTION OF THE FIGURES

FIG. 1: Schematic illustration of the At-line monitoring of a bioprocess with the inventive electrical DNA chips

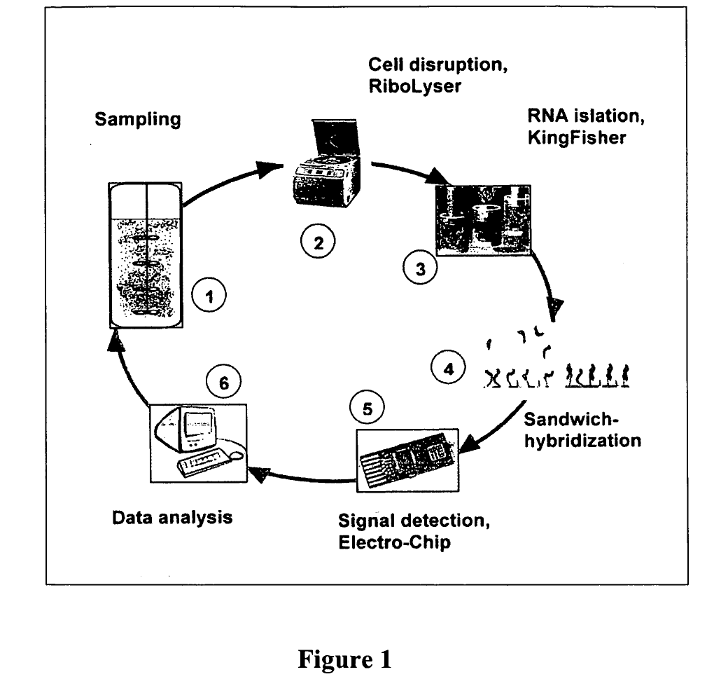

The real-time monitoring of the bioprocess advantageously involves the following steps:
1. Sampling, for example from the fermentation of a microorganism;
2. Cell disruption by routine methods;
3. RNA isolation by routine methods;
4. Hybridization on a chip inventively loaded with nucleic acids (for example DNA) or nucleic acid analogs (for example difficultly hydrolyzable, analogously structured compounds);
5. Acquisition of the electrical signals from a suitably constructed electrochip; alternatively, the recording of optical signals from an optical DNA chip would also be possible;
6. Preferably computer-supported data analysis.
   When electrical chips are used, an approximate total analysis time is less than 2 hours under the present state of development; with conventional optical DNA chips it takes about 12 hours.

1. A nucleic acid-binding chip, comprising probes for at least three of:
kdgR, citA, gene coding for a putative protein (putative ABC transporter/amino acid permease) (homolog to SEQ ID NO. 91), htrA, ycnl, gene coding for a hypothetical protein of unknown function (homolog to SEQ ID NO. 41), gene coding for a hypothetical protein of unknown function (homolog to SEQ ID NO. 53), yppF, yqjN, ggt, alsR, glnA, nrgA, yciC, yvtA, nrgB, gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), or tnrA,
wherein the total number of probes is not greater than 80.

2. The nucleic acid-binding chip according to claim 1, wherein the probes are selected from, in order of preference: kdgR, citA, gene coding for a putative protein (putative ABC transporter/amino acid permease) (homolog to SEQ ID NO. 91), htrA, ycnI, gene coding for a hypothetical protein of unknown function (homolog to SEQ ID NO. 41), gene coding for a hypothetical protein of unknown function (homolog to SEQ ID NO. 531 yppF, yqjN, ggt, alsR, glnA, nrgA, yciC, yvtA, nrgB, gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), and tnrA.

3. The nucleic acid-binding chip according to claim 1, wherein at least one of the probes is:
gene coding for a conserved hypothetical protein of unknown function (homolog to SEQ ID NO. 19), gene coding for a putative protein (ATP-binding protein of a putative ABC-transporter) (homolog to SEQ ID NO. 55), ycnJ, glnR, yvlA, yncE, yvlB, gene coding for a putative protein (putative hydrolase) (homolog to SEQ ID NO. 47), trpF, ydfS, trpD, gene coding for a putative protein (putative phage capsid protein) (homolog to SEQ ID NO. 21), ycnK, trpB, trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), or tnrA.

4. The nucleic acid-binding chip according to claim 1 comprising at least 4 of the specified probes.

5. The nucleic acid-binding chip according to claim 1, wherein the total number of probes does not exceed 75, 70, 65, 60, 55, 50, 40, 30, 20 or 10.

6. (canceled)

7. (canceled)

8. The nucleic acid-binding chip according to claim 53, wherein the unicellular eukaryotes are protozoa or fungi.

9. The nucleic acid-binding chip according to claim 53, wherein the Gram-positive bacteria are Coryneform bacteria or a species of the genera Staphylococcus, Corynebacteria or Bacillus.

10. The nucleic acid-binding chip according to claim 53, wherein the Gram-negative bacteria are a species of the genera Escherichia or Klebsiella.

11. (canceled)

12. (canceled)

13. The nucleic acid-binding chip according to claim 1 further comprising at least one probe for detecting a gene encoding an amylase, cellulase, lipase, oxidoreductase, hemicellulase, or protease.

14. The nucleic acid-binding chip according to claim 1, wherein at least one of the probes is single-stranded.

15. The nucleic acid-binding chip according to claim 1, wherein at least one of the probes is DNA or a nucleic acid analog.

16. The nucleic acid-binding chip according to claim 1, wherein at least one of the probes comprises a region that is transcribed.

17. The nucleic acid-binding chip according to claim 1, wherein at least one of the probes is capable of binding to fragments of the gene of interest.

18. The nucleic acid-binding chip according to claim 1, wherein at least one of the probes is less than 200 nucleotides long.

19. The nucleic acid-binding chip according to claim 1, wherein an electric signal is triggered by the specific binding of mRNA to one or more probes.

20-27. (canceled)

28. A method for determining the physiological state of a unicellular eukaryote, a Gram positive bacteria, or a Gram negative bacteria under nitrogen deficiency conditions, comprising expression profiling the unicellular eukaryote, Gram positive bacteria, or Gram negative bacteria with the nucleic acid-binding chip of claim 1.

29. (canceled)

30. (canceled)

31. The method according to claim 28, wherein the unicellular eukaryote is a protozoa or fungi.

32. The method according to claim 28, wherein the Gram-positive bacteria are Coryneform bacteria or a species of the genera Staphylococcus, Corynebacteria or Bacillus.

33. The method according to claim 28, wherein the Gram-negative bacteria are a species of the genera Escherichia or Klebsiella.

34. The method according to claim 28, wherein the chip comprises probes derived from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97 or 99.

35. The method according to claim 28, wherein the physiological state is determined at various times during nitrogen deficiency conditions.

36. The method according to claim 28, wherein the nitrogen deficiency condition occurs during fermentation.

37. The method according to claim 36, wherein the fermentation occurs during the production of a food or beverage product, nutritional supplement, or pharmaceutical by the unicellular eukaryote, Gram positive bacteria, or Gram negative bacteria.

38. The method according to claim 36, wherein the fermentation occurs during the expression of an α-amylase, protease, cellulase, lipase, oxidoreductase, peroxidase, laccase, oxidase or hemicellulase by the unicellular eukaryote, Gram positive bacteria, or Gram negative bacteria.

39-49. (canceled)

50. The nucleic acid binding chip according to claim 1, wherein at least one of the probes is trpC, gene coding for a putative protein (putative transcription regulator) (homolog to SEQ ID NO. 17), gene coding for a putative protein (putative serine protease) (homolog to SEQ ID NO. 9), gene coding for a putative protein (putative glycosyl hydrolase/lysozyme) (homolog to SEQ ID NO. 85), gene coding for a putative protein (putative malate synthase, EC 4.1.3.2) (homolog to SEQ ID NO. 45), gene coding for a putative protein (putative Na(+)-bonded D-alanine-glycine permease) (homolog to SEQ ID NO. 49), gene coding for a hypothetical protein of unknown function (homolog to SEQ ID NO. 95), nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, gene coding for a hypothetical protein of unknown function (homolog to SEQ ID NO. 81), or tnrA.

51. The nucleic acid binding chip according to claim 1, wherein at least one of the probes is nasD, gene coding for a putative protein (putative ammonium transporter) (homolog to SEQ ID NO. 63), ycdH, nasC, gene coding for a hypothetical protein (close homolog to the aldehyde dehydrogenase DhaS) (homolog to SEQ ID NO. 15), nasB, trpE, pckA, gene coding for a putative protein (putative nitrogen regulation protein P-II) (homolog to SEQ ID NO. 93), nasF, yrkC, or gene coding for a hypothetical protein of unknown function (homolog to SEQ ID NO. 81).

52. The nucleic acid binding chip according to claim 1, wherein at least one of the probes is the gene coding for a putative protein (putative nitrogen regulation protein P-II) (homolog to SEQ ID NO. 93), nasF, yrkC, or gene coding for a hypothetical protein of unknown function (homolog to SEQ ID NO. 81).

53. The nucleic acid binding chip according to claim 1, wherein the probes correspond to a gene that exhibits at least an eight-fold change in expression in a unicellular eukaryote, Gram positive bacteria, or Gram negative bacteria in response to nitrogen deficiency conditions.

54. The nucleic acid binding chip according to claim 8, wherein the fungi are a species of Saccharomyces or Schizosaccharomyces.

55. The nucleic acid binding chip according to claim 9, wherein the Gram positive bacteria are Staphylococcus carnosus, Corynebacterium glutamicum, Bacillus subtilis, B. licheniformis, B. amyloliquefaciens, B. agaradherens, B. stearothermophilus, B. globigii B. lentus, or B. licheniformis.

56. The nucleic acid binding chip according to claim 10, wherein the Gram negative bacteria are derivatives of Escherichia coli K12, of Escherichia coli B, Klebsiella planticola, derivatives of Escherichia coli BL21 (DE3), E. coli RV308, E. coli DH5α, E. coli JM109, E. coli XL-1 or Klebsiella planticola (Rf).

57. The nucleic acid binding chip according to claim 1, wherein at least one of the probes is less than 100 nucleotides long.

58. The nucleic acid binding chip according to claim 1, wherein at least one of the probes is 20 to 60 nucleotides long.

59. The nucleic acid binding chip according to claim 1, wherein at least one of the probes is 45 to 55 nucleotides long.

60. The method according to claim 31, wherein the fungi are a species of Saccharomyces or Schizosaccharomyces.

61. The method according to claim 32, wherein the Gram positive bacteria are Staphylococcus carnosus, Corynebacterium glutamicum, Bacillus subtilis, B. licheniformis, B. amyloliquefaciens, B. agaradherens, B. stearothermophilus, B. globigii B. lentus, or B. licheniformis.

62. The method according to claim 33, wherein the Gram negative bacteria are derivatives of Escherichia coli K12, of Escherichia coli B, Klebsiella planticola, derivatives of Escherichia coli BL21 (DE3), E. coli RV308, E. coli DH5α, E. coli JM109, E. coli XL-1 or Klebsiella planticola (Rf).
